SEQUENCE LISTING (86 sequences)

SEQ ID NO: 1 - protein, 157 aa, synthetic construct
  REGION 1..157: note = DARPin Monomer Protein
DLGKKLLEAARAGQDDEVRILMANGADVNARDESGTTPLHLAAGSGHLEIVEVLLKHGADVNAADLGGWTPLHLAAIWGHLEIVEVLLKHGADVNATDPTGWTPLHLAALGGHLEIVEVLLKNGADVNAQDKFGKTAFDISIDNGNEDLAEILQSSS

SEQ ID NO: 2 - protein, 190 aa, synthetic construct
  REGION 1..190: note = DARPin Monomer Protein
DLGKKLLEAARAGQDDEVRILMANGADVNACDSNGRTPLHLAAVRGHLEIVEVLLKNGADVNAWDVIGKTPLHLAADGGHLEIVEVLLKNGADVNAWDLIGRTPLHLAALDGHLEIVEVLLEHGADVNAYDRPGHTPLHLAARRGHLEIVEVLLKYGADVNAQDKFGKTAFDISIDNGNEDLAEILQSSS

SEQ ID NO: 3 - protein, 157 aa, synthetic construct
  REGION 1..157: note = DARPin Monomer Protein
DLGKKLLEAARAGQDDEVRILMANGADVNAPDEYGSTPLHLAASLGHLEIVEVLLKHGADVNATDRLGNTPLHLAALGGHLEIVEVLLKYGADVNAADPAGWTPLHLAALGGHLEIVEVLLKYGADVNAQDKFGKTAFDISIDNGNEDLAEILQSSS

SEQ ID NO: 4 - protein, 157 aa, synthetic construct
  REGION 1..157: note = DARPin Monomer Protein
DLGKKLLEAARAGQDDEVRILMANGADVNACDAGGTTPLHLAASGGHLEIVEVLLKNGADVNAYDPGGWTPLHLAAIRGHLEIVEVLLKYGADVNAEDPLGWTPLHLAALAGHLEIVEVLLKNGADVNAQDKFGKTAFDISIDNGNEDLAEILQSSS

SEQ ID NO: 5 - protein, 157 aa, synthetic construct
  REGION 1..157: note = DARPin Monomer Protein
DLGKKLLEAARAGQDDEVRILMANGADVNAGDPFGFTPLHLAAMFGHLEIVEVLLKNGADVNAHDEYGFTPLHLAAVVGHLEIVEVLLKNGADVNACDNQGGTPLHLAAVDGHLEIVEVLLKHGADVNAQDKFGKTAFDISIDNGNEDLAEILQSSS

SEQ ID NO: 6 - protein, 157 aa, synthetic construct
  REGION 1..157: note = DARPin Monomer Protein
DLGKKLLEAARAGQDDEVRILMANGADVNASDSVGWTPLHLAADRGHLEIVEVLLKNGADVNAYDPGGWTPLHLAAIRGHLEIVEVLLKYGADVNAEDPLGWTPLHLAALAGHLEIVEVLLKNGADVNAQDKFGKTAFDISIDNGNEDLAEILQSSS

SEQ ID NO: 7 - protein, 157 aa, synthetic construct
  REGION 1..157: note = DARPin Monomer Protein
DLGKKLLEAARAGQDDEVRILMANGADVNAMDRHGQTPLHLAASFGHLEIVEVLLKHGADVNANDYSGGTPLHLAAIWGHLEIVGVLLKNGADVNAKDVWGRTPLHLAAIGGHLEIVEVLLKNGADVNAQDKFGKTAFDISIDNGNEDLAEILQSSS

SEQ ID NO: 8 - protein, 157 aa, synthetic construct
  REGION 1..157: note = DARPin Monomer Protein
DLGKKLLEAARAGQDDEVRILMANGADVNARDHLGHTPLHLAASRGHLEIVEVLLKHGADVNASDSVGQTPLHLAAVWGHLEIVEVLLKNGADVNALDTLGWTPLHLAALGGHLEIVEVLLKHGADVNAQDKFGKTAFDISIDNGNEDLAEILQSSS

SEQ ID NO: 9 - protein, 157 aa, synthetic construct
  REGION 1..157: note = DARPin Monomer Protein
DLGKKLLEAARAGQDDEVRILMANGADVNARDFPGLTPLHLAAVQGHLEIVEVLLKNGADVNAVDKLGLTPLHLAARRGHLEIVEVLLKHGADVNAVDYTGTTPLNLAAVPGHLEIVEVLLKNGADVNAQDKFGKTAFDISIDNGNEDLAEILQSSS

SEQ ID NO: 10 - protein, 157 aa, synthetic construct
  REGION 1..157: note = DARPin Monomer Protein
DLGKKLLEAARAGQDDEVRILMANGADVNAMDRHGQTPLHLAASFGRLEIVEVLLKHGADVNANDYSGGTPLHLAAIWGHLEIVGVLLKNGADVNAKDVWGRTPLHLAAIGGHLEIVEVLLKNGADVNAQDKFGKTAFDISIDNGNEDLAEILQSSS

SEQ ID NO: 11 - protein, 157 aa, synthetic construct
  REGION 1..157: note = DARPin Monomer Protein
DLGKKLLEAARAGQDDEVRILMANGADVNASDSVGWTPLHLAADRGHLEIVEVLLKNGADVNAYDPGGWTPLHLAAIRGHLEIVEVLLKYGADVNAEDPLGWTPLHLAALAGHLEIVEVLLRNGADVNAQDKFGKTAFDISIDNGNEDLAEILQSSS

SEQ ID NO: 12 - protein, 157 aa, synthetic construct
  REGION 1..157: note = DARPin Monomer Protein
DLGKKLLEAARAGQDDEVRILMANGADVNARDFPGLTPLHLAAVQGHLEIVEVLLKNGADVNAVDKLGLTPLHLAARRGHLEIVEVLLKHGADVNAVDYTGTTPLHLAAVPGHLEIVEVLLKNGADVNAQDKFGKTAFDISIDNGNEDLAEILQSSS

SEQ ID NO: 13 - protein, 157 aa, synthetic construct
  REGION 1..157: note = DARPin Monomer Protein
DLGKKLLEAARAGQDDEVRILVANGADVNAPDEYGSTPLHLAASLGHLEIVEVLLKHGADVNATDRLGNTPLHLAALGGHLEIVEVLLKYGADVNAADPAGWTPLHLAALGGHLEIVEVLLKYGADVNAQDKFGKTAFDISIDNGNEDLAEILQSSS

SEQ ID NO: 14 - protein, 157 aa, synthetic construct
  REGION 1..157: note = DARPin Monomer Protein
DLGKKLLEAARAGQDDEVRILMANGADVNAKDRHGQTPLHLAASFGHLEIVEVLLKHGADVNANDYSGGTPLHLAAIWGHLEIVGVLLKNGADVNAKDVWGRTPLHLAAIGGHLEIVEVLLKNGADVNAQDKFGKTAFDISIDNGNEDLAEILQSSS

SEQ ID NO: 15 - protein, 157 aa, synthetic construct
  REGION 1..157: note = DARPin Monomer Protein
DLGKKLLEAARAGQDDEVRILMANGADVNAADPAGWTPLHLAACYGHLEIVGVLLKNGADVNALDWGGWTPLHLAAVIGHLEIVGVLLKHGADVNATDLSGWTPLHLAAFRGHLEIVEVLLKHGADVNAQDKFGKTAFDISIDNGNEDLAEILQSSS

SEQ ID NO: 16 - protein, 157 aa, synthetic construct
  REGION 1..157: note = DARPin Monomer Protein
DLGKKLLVAARAGQDGEVRILMANGADVNASDSVGWTPLHLAADRGHLEIVEVLLKNGADVNAYDPGGWTPLHLAAIRGHLEIVEVLLKYGADVNAEDPLGWTPLHLAALAGHLEIVEVLLKNGADVNAQDKFGKTAFDISIDNGNEDLAEILQSSS

SEQ ID NO: 17 - protein, 157 aa, synthetic construct
  REGION 1..157: note = DARPin Monomer Protein
DLGKKLLEAARAGQDDEVRILMANGADVNAGDPFGFTPLHLAAMFGHLEIVEVLLKNGADVNAHEEYGFTPLHLAAVVGHLEIVEVLLNNGADVNACDNQGGTPLHLAAHTGHLEIVEVLLKHGADVNAQDKFGKTAFDISIDIGNEDLAEILQSSS

SEQ ID NO: 18 - protein, 157 aa, synthetic construct
  REGION 1..157: note = DARPin Monomer Protein
DLGKKLLVAARAGQDDEVRILMANGADVNAGDPFGFTPLHLAAMFGHLEIVEVLLKNGADVNAHEEYGFTPLHLAAVVGHLEIVEVLLKNGADVNACDNQGGTPLHLAAHTGHLEIVEVLLKHGADVNAQDKFGKTAFDISIDNGNEDLAEILQSSS

SEQ ID NO: 19 - protein, 157 aa, synthetic construct
  REGION 1..157: note = DARPin Monomer Protein
DLGKKLLEAARAGQDDEVRILMANGADVNAGDPFGFTPLHLAAMFGHLEIVEVLLKNGADVNAHEEYGFTPLHLAAVVSHMEIVEVLLKNGADVNACDNQGGTPLHLAAHTGHLEIVEVLLKHGADVNAQDKFGKTAFDISIDNGNEDLAEILQSSS

SEQ ID NO: 20 - protein, 157 aa, synthetic construct
  REGION 1..157: note = DARPin Monomer Protein
DLGKKLLEAARAGQDDEVRILMANGADVNAGDPFGFTPLHLAAMFGHLEIVEVLLKNGADVNAHEEYGFTPLHLAAVVGHLEIVEVLLKNGADVNACDNQGGTPLHLAARMGHLEMVEVLLKHGADVNAQDKFGKTAFDISIDNGNEDLAEILQSSS

SEQ ID NO: 21 - protein, 157 aa, synthetic construct
  REGION 1..157: note = DARPin Monomer Protein
DLGKKLLEAARAGQDDEVRILMANGADVNAGDPFGFTPLHLAAMFGHLEIVEVLLKNGADVNAHEEYGFTPLHLAAVVGHLEIVEVLLKNGADVNACDNQGGTPLHLAARIGHLEIVEVLLKHGADVNAQDKFGKTAFDISIDNGNEDLAEILQSSS

SEQ ID NO: 22 - protein, 157 aa, synthetic construct
  REGION 1..157: note = DARPin Monomer Protein
DLGKKLLEAARAGQDDEVRILVANGADVNAGDPFGFTPLHLAAMFGHLEIVEVLLKNGADVNAHEEYGFTPLHLAAVVGHLEIVEVLLRNGADVNACDNQGGTPLHLAAHTGHLEIVEVLLKHGADVNAQDKFGKTAFDISIDNGNEDLAEILQSSS

SEQ ID NO: 23 - protein, 157 aa, synthetic construct
  REGION 1..157: note = DARPin Monomer Protein
DLGKKLLEAARAGQDDEVRILMANGADVNAGDPFGFTPLHLAAMFGHLEIVEVLLKNGADVNAHEEYGFTPLHLAAVVGHLEIVEVLLKNGADVNACDNQGGTPLHLAAHTGHLEIVEVLLKHGADVNAQDKFGKTAFDISIDNGNEDLAEILQSSS

SEQ ID NO: 24 - protein, 157 aa, synthetic construct
  REGION 1..157: note = DARPin Monomer Protein
DLGKKLLEAARAGQDDEVRILMANGADVNAGDPFGFTPLHLAAMFGHLEIVEVLLKNGADVNAHEEYGFTPLHLAAVVGLLEIVEVLLKNGADVNACDNQGGTPLHLAAHTGHLEIVEVLLKHGADVNAQDKFGKTAFDISIGYGNEDLAEILQSSS

SEQ ID NO: 25 - protein, 157 aa, synthetic construct
  REGION 1..157: note = DARPin Monomer Protein
DLGKKLLEAARAGQDDEVRILMANGADVNVGDPFGFTPLHLAAMFGHLEIVEVLLKNGADVNAHEEYGFTPLHLAAVVGHLEIVEVLLKNGADVNACDNQGGTPLHLAAHTGHLEIVEVLLKHGADVNAQDKFGKTAFDISIDNGNEDLAEILQSSS

SEQ ID NO: 26 - protein, 157 aa, synthetic construct
  REGION 1..157: note = DARPin Monomer Protein
DLGKKLLEAARAGQDDEVRILMANGADVNAGDPFGFTPLHLAALYGHLEIVEVLLKNGADVNAHEEYGFTPLHLAAVVGHLEIVEVLLKNGADVNACDNQGGTPLHLAAHTGHLEIVEVLLKHGADVNAQDKFGKTAFDISIDNGNEDLAEILQSSS

SEQ ID NO: 27 - protein, 157 aa, synthetic construct
  REGION 1..157: note = DARPin Monomer Protein
DLGKKLLEAARAGQDDEVRILMANGADVNAGDPFGFTPLHLAAMYGHLEIVEVLLKNGADVNAHEEYGFTPLHLAAVVGHLEIVEVLLKNGADVNACDNQGGTPLHLAAHTGHLEIVEVLLKHGADVNAQDKFGKTAYDISIDNGNEDLAEILQSSS

SEQ ID NO: 28 - protein, 157 aa, synthetic construct
  REGION 1..157: note = DARPin Monomer Protein
DLGKKLLEAARAGQDDVVRILMANGADVNAGDPFGFTPLHLAAMFGHLEIAEVLSKNGADVNAHEEYGFTPLHLAAVVGHLGIVEVLLKNGADVNACDNQGGTPLHLAARIGHLEIVEVLLKHGADVNAQDKFGKTAFDISIDNGNEDLAEILQSSS

SEQ ID NO: 29 - protein, 157 aa, synthetic construct
  REGION 1..157: note = DARPin Monomer Protein
DLGKKLLDAARTGQDDEVRILMANGADVNAGDPFGFTPLHLAALFGHLEIVEVLLKNGADVNAHEEYGFTPLHLAAVVGHLEIVEVLLKNGADVNACDNQGGTPLHLAARIGHLEIVEVLLKQGADVNAQDKFGKTAFDISIDNGNEDLAEILQSSS

SEQ ID NO: 30 - protein, 157 aa, synthetic construct
  REGION 1..157: note = DARPin Monomer Protein
DLGKKLLEAARAGQDDEVRILMANGADVNAGDPFGFTPLHLAAMFGHLEIVEVLLMNGADVNAHEEYGFTPLHLAAVVGHLEIVGVLLKNGADVNASDNQGGTPLHLAARIGHLEIVEVLLKHGADVNAQDKFGKTAFDISIDNGNEDLAEILQSSS

SEQ ID NO: 31 - protein, 157 aa, synthetic construct
  REGION 1..157: note = DARPin Monomer Protein
DLGKKLLEAARAGQDDEVRILMANGADVNAGDPFGFTPLHLAAMFGHLGIVEVLLKNGADVNAHEEYGFTPLHLAAVVGHLEIVEVLLKNGADVNACDNQGGTPLHLAARIGHLEIVEVLLKHGADVNAQDKFGKTAFDISIDIGNEDLAEILQSSS

SEQ ID NO: 32 - protein, 157 aa, synthetic construct
  REGION 1..157: note = DARPin Monomer Protein
DLGKKLLEAARAGQDDEVRILMANGADVNAGDPFGFTPLHFAAMFGHLEIVEVLLKNGADVNAHEEYGFTPLHLAAVVGHLEIVEVLLKNGADVNACDNQGGTPLHLAAHTGHLEIVEVLLKHGADVNAQDKFGKTAFDISIDIGNEDLAEILQSSS

SEQ ID NO: 33 - protein, 157 aa, synthetic construct
  REGION 1..157: note = DARPin Monomer Protein
DLGKKLLEAARAGQDDEVRTLMANGADVNAGDPFGFTPLHLAAMYGHLEIVEVLLKNGADVNAHEEYGFTPLHLAAVVGHLEIVEVLLKNGADVNACDNQGGTPLHLAAHTGHLEIVEVLLKHGADVNAQDKFGKTAFDISIDNGNEDLAEILQSSS

SEQ ID NO: 34 - protein, 157 aa, synthetic construct
  REGION 1..157: note = DARPin Monomer Protein
DLGKKLLEAARAGQDDEVRILMANGADVNAGDPFGFTPLHLAAMFGHLEIVEVLLKNGAGVNAHEEYGFTPLHLAAVVGHLEIVEVLLKNGADVNACDNQGGTPLHLAAHTGHLEIVEVLLKHGADVNAQDKFGKTAFDISIDNGNEDLAEILQSSS

SEQ ID NO: 35 - protein, 157 aa, synthetic construct
  REGION 1..157: note = DARPin Monomer Protein
DLGKKLLEAARAGQDDEVRILMANGADVNACDPFGFTPLHLAAMFGHLEIVEVLLKNGADVNAHEEYGFTPLHLAAVVGHLEILEVLLKNGADVNARDNQGGSPLHLAAHTGHLEIVEVLLKRGADVNAQDKFGKTAFDISIDNGNEDLAEILQSSS

SEQ ID NO: 36 - protein, 157 aa, synthetic construct
  REGION 1..157: note = DARPin Monomer Protein
DLGKKLLEAARAGQDDEVRILMANGADVNAGDPFGFTPLHLAAMFGHLEIVEVLLKNGADVNAHEEYGFTPLHLAAVVGHLEIVEVLLNNGADVNACDNQGGTPLHLAAHTGHLEIVEVLLKHGADVNAQDKFGKTAFDISIDNGNEDLAEILQSSS

SEQ ID NO: 37 - protein, 157 aa, synthetic construct
  REGION 1..157: note = DARPin Monomer Protein
DLGKKLLEAARAGQDDEVRILMANGADVNAGDPFGFTPLHLAAMFGHLEIVEVLMKNGADANAHEEYGFTPLHLAAVVGHLEIVEVLLKNGADVNACDNQGGTPLHLAAHTGHLEIVEVLLKHGADVNAQDKFGKTAFDISIDNGNEDLAEILQSSS

SEQ ID NO: 38 - protein, 157 aa, synthetic construct
  REGION 1..157: note = DARPin Monomer Protein
DLGKKLLEAARAGQDDEVRILMANGADVNAGDPFGFTPLHLAAMFGHLEIVEVLLKNGADVNAHEEYGFTPLHLAAVVGHLEIVEVLLKNGADVNACDNQGGTPLHLASHTGHLEIVEVLLKHGADVNAQDKFGKTAFDISIDTGNEDLAEILQSSS

SEQ ID NO: 39 - protein, 157 aa, synthetic construct
  REGION 1..157: note = DARPin Monomer Protein
DLGKKLLEAARAGQDDEVRFLMANGADVNAGDPFGFTPLHLAAMFGHLEIVEVLLKNGADENAHEEYGFTPLHLAAVVGHLEIVEVLLKNGADVNACDNQGGTPLHLAAHTGHLEIVEVLLKHGADVNAQDKFGKTAFDISIDNGNEDLAEILQSSS

SEQ ID NO: 40 - protein, 157 aa, synthetic construct
  REGION 1..157: note = DARPin Monomer Protein
DLGKKLLEAARAGQDDEVRILMANGADVNAGDPFGFTPLHLAAMYGHLEIVEVLLKNGADVNAHEEYGFTPLHLAAVVGHLEIVEVLLKNGADVNACDNQGGTPLHLAARIGHLEIVEVLLKHGADVNAQDKFGKTAFDISIDNGNEDLAEILQSSS

SEQ ID NO: 41 - protein, 157 aa, synthetic construct
  REGION 1..157: note = DARPin Monomer Protein
DLGKKLLEAARAGQDDEVRILMANGADVNAGDPFGFTPLHLAAMFGHLEIVEVLLKNGADVNAHDEYGFTPLHLAAVVGHLEIVEVLLKNGADVNACDNQGGTPLHLAAHTGHLEIVEVLLKHGADVNAQDKFGKTAFDISIDNGNEDLAEILQSSS

SEQ ID NO: 42 - protein, 157 aa, synthetic construct
  REGION 1..157: note = DARPin Monomer Protein
DLGKKLLEAARAGQDDEVRILMANGADVNAGDPFGFTPLHLAAMFGHLEIVEVLLKNGADVNAHDEYGFTPLHLAAVVGHLEIVEVLLKNGADVNACDNQGGTPLHLAARIGHLEIVEVLLKHGADVNAQDKFGKTAFDISIDNGNEDLAEILQSSS

SEQ ID NO: 43 - protein, 157 aa, synthetic construct
  REGION 1..157: note = DARPin Monomer Protein
DLGKKLLEAARAGQDDEVRILMANGADVNAGDPFGFTPLHLAAMYGHLEIVEVLLKNGADVNAHEEYGFTPLHLAAVVGHLEIVEVLLKNGADVNACDNQGGTPLHLAAHTGHLEIVEVLLKHGADVNAQDKFGKTAFDISIDNGNEDLAEILQSSS

SEQ ID NO: 44 - protein, 157 aa, synthetic construct
  REGION 1..157: note = DARPin Monomer Protein
DLGKKLLEAARAGQDDEVRILMANGADVNAGDPFGFTPLHLAAMFGHLEIVEVLLKNGADVNAHEEYGFTPLHLAAVVGHLEIVEVLLKNGADVNACDNQGGTPLHLAAHTGHLEIVEVLLKHGADVNAQDKFGKTAFDISIDIGNEDLAEILQSSS

SEQ ID NO: 45 - protein, 157 aa, synthetic construct
  REGION 1..157: note = DARPin Monomer Protein
DLGKKLLEAARAGQDDEVRILMANGADVNAGDPFGFTPLHLAAMFGHLEIVEVLLKNGADVNAHDEYGFTPLHLAAVVGHLEIVEVLLKNGADVNACDNQGGTPLHLAARMGHLEIVEVLLKHGADVNAQDKFGKTAFDISIDNGNEDLAEILQSSS

SEQ ID NO: 46 - protein, 157 aa, synthetic construct
  REGION 1..157: note = DARPin Monomer Protein
DLGKKLLEAARAGQDDEVRILMANGADVNAGDPFGFTPLHLAAMFGHLEIVEVLLKNGADVNAHEEYGFTPLHLAAVVGHLEIVEVLLKNGADVNACDNQGGTPLHLAARMGHLEIVEVLLKHGADVNAQDKFGKTAFDISIDNGNEDLAEILQSSS

SEQ ID NO: 47 - protein, 157 aa, synthetic construct
  REGION 1..157: note = DARPin Monomer Protein
DLGKKLLEAARAGQDDEVRILMANGADVNAGDPFGFTPLHLAAMFGHLEIVEVLLKNGADVNAHEEYGFTPLHLAAVVGHLEIVEVLLKNGADVNACDNQGGTPLHLAAHTGHLEIVEVLLKHGADVNAQDKFGKTAFDISIDTGNEDLAEILQSSS

SEQ ID NO: 48 - protein, 157 aa, synthetic construct
  REGION 1..157: note = DARPin Monomer Protein
DLGKKLLEAARAGQDDEVRILMANGADVNAGDPFGFTPLHLAAMFGHLEIVEVLLKNGADVNAHEEYGFTPLHLAAVVGHLEIVEVLLKNGADVNARDNQGGSPLHLAAHTGHLEIVEVLLKHGADVNAQDKFGKTAFDISIDNGNEDLAEILQSSS

SEQ ID NO: 49 - protein, 157 aa, synthetic construct
  REGION 1..157: note = DARPin Monomer Protein
DLGKKLLEAARAGQDDEVRILVANGADVNAGDPFGFTPLHLAALYGHLEIVEVLLKNGADVNAHEEYGFTPLHLAAVVSHMEIVEVLLNNGADVNARDNQGGSPLHLASHTGHLEIVEVLLKQGADVNAQDKFGKTAYDISIDIGNEDLAEILQSSS

SEQ ID NO: 50 - protein, 157 aa, synthetic construct
  REGION 1..157: note = DARPin Monomer Protein
DLGKKLLEAARAGQDDEVRILMANGADVNAGDPFGFTPLHLAAMFGHLEIVEVLLKNGADVNAHEEYGFTPLHLAAVVGHLEIVEVLLKNGADVNACDNQGGTPLHLAARIGHLEIVEVLLKQGADVNAQDKFGKTAFDISIDNGNEDLAEILQSSS

SEQ ID NO: 51 - protein, 312 aa, synthetic construct
  VARIANT 155: note = X can be any amino acid
  REGION 1..312: note = DARPin Dimer Protein
DLGKKLLEAARAGQDDEVRILMANGADVNAADPAGWTPLHLAACYGHLEIVGVLLKNGADVNALDWGGWTPLHLAAVIGHLEIVGVLLKHGADVNATDLSGWTPLHLAAFRGHLEIVEVLLKHGADVNAQDKFGKTAFDISIDNGNEDLAEILQXDLGKKLLEAARAGQDDEVRILMANGADVNARDFPGLTPLHLAAVQGHLEIVEVLLKNGADVNAVDKLGLTPLHLAARRGHLEIVEVLLKHGADVNAVDYTGTTPLHLAAVPGHLEIVEVLLKNGADVNAQDKFGKTAFDISIDNGNEDLAEILQSSS

SEQ ID NO: 52 - protein, 312 aa, synthetic construct
  VARIANT 155: note = X can be any amino acid
  REGION 1..312: note = DARPin Dimer Protein
DLGKKLLEAARAGQDDEVRILMANGADVNARDFPGLTPLHLAAVQGHLEIVDVLLKNGADVNAVDKLGLTPLHLAARRGHLEIVEVLLKHGADVNAVDYTGTTPLHLAAVPGHLEIVEVLLKNGADVNAQDKFGKTAFDISIDNGNEDLAEILQXDLGKKLLEAARAGQDDEVRILMANGADVNAGDPFGFTPLHLAAMFGHLEIVEVLLKNGADVNAHDEYGFTPLHLAAVVGHLEIVEVLLKNGADVNACDNQGGTPLHLAARIGHLEIVEVLLKHGADVNAQDKFGKTAFDISIDNGNEDLAEILQSSS

SEQ ID NO: 53 - protein, 312 aa, synthetic construct; identical to SEQ ID NO: 52
  VARIANT 155: note = X can be any amino acid
  REGION 1..312: note = DARPin Dimer Protein

SEQ ID NO: 54 - protein, 312 aa, synthetic construct; identical to SEQ ID NO: 52
  VARIANT 155: note = X can be any amino acid
  REGION 1..312: note = DARPin Dimer Protein

SEQ ID NO: 55 - protein, 345 aa, synthetic construct
  VARIANT 188: note = X can be any amino acid
  REGION 1..345: note = DARPin Dimer Protein
DLGKKLLEAARAGQDDEVRILMANGADVNACDSNGRTPLHLAAVRGHLEIVEVLLKNGADVNAWDVIGKTPLHLAADGGHLEIVEVLLKNGADVNAWDLIGRTPLHLAALDGHLEIVEVLLEHGADVNAYDRPGHTPLHLAARRGHLEIVEVLLKYGADVNAQDKFGKTAFDISIDNGNEDLAEILQXDLGKKLLEAARAGQDDEVRILMANGADVNAGDPFGFTPLHLAAMFGHLEIVEVLLKNGADVNAHDEYGFTPLHLAAVVGHLEIVEVLLKNGADVNACDNQGGTPLHLAAHTGHLEIVEVLLKHGADVNAQDKFGKTAFDISIDNGNEDLAEILQSSS

SEQ ID NO: 56 - protein, 312 aa, synthetic construct
  VARIANT 155: note = X can be any amino acid
  REGION 1..312: note = DARPin Dimer Protein
DLGKKLLEAARAGQDDEVRILMANGADVNARDFPGLTPLHLAAVQGHLEIVEVLLKNGADVNAVDKLGLTPLHLAARRGHLEIVEVLLKHGADVNAVDYTGTTPLNLAAVPGHLEIVEVLLKNGADVNAQDKFGKTAFDISIDNGNEDLAEILQXDLGKKLLEAARAGQDDEVRILMANGADVNAGDPFGFTPLHLAAMFGHLEIVEVLLKNGADVNAHDEYGFTPLHLAAVVGHLEIVEVLLKNGADVNACDNQGGTPLHLAARIGHLEIVEVLLKHGADVNAQDKFGKTAFDISIDNGNEDLAEILQSSS

SEQ ID NO: 57 - protein, 312 aa, synthetic construct
  VARIANT 155: note = X can be any amino acid
  REGION 1..312: note = DARPin Dimer Protein
DLGKKLLEAARAGQDDEVRILMANGADVNAGDPFGFTPLHLAAMFGHLEIVEVLLKNGADVNAHDEYGFTPLHLAAVVGHLEIVEVLLKNGADVNACDNQGGTPLHLAARIGHLEIVEVLLKHGADVNAQDKFGKTAFDISIDNGNEDLAEILQXDLGKKLLEAARAGQDDEVRILMANGADVNAMDRHGQTPLHLAASFGHLEIVEVLLKHGADVNANDYSGGTPLHLAAIWGHLEIVGVLLKNGADVNAKDVWGRTPLHLAAIGGHLEIVEVLLKNGADVNAQDKFGKTAFDISIDNGNEDLAEILQSSS

SEQ ID NO: 58 - protein, 312 aa, synthetic construct
  VARIANT 155: note = X can be any amino acid
  REGION 1..312: note = DARPin Dimer Protein
DLGKKLLEAARAGQDDEVRILMANGADVNAGDPFGFTPLHLAAMFGHLEIVEVLLKNGADVNAHDEYGFTPLHLAAVVGHLEIVEVLLKNGADVNACDNQGGTPLHLAARIGHLEIVEVLLKHGADVNAQDKFGKTAFDISIDNGNEDLAEILQXDLGKKLLEAARAGQDDEVRILMANGADVNARDESGTTPLHLAAGSGHLEIVEVLLKHGADVNAADLGGWTPLHLAAIWGHLEIVEVLLKHGADVNATDPTGWTPLHLAALGGHLEIVEVLLKNGADVNAQDKFGKTAFDISIDNGNEDLAEILQSSS

SEQ ID NO: 59 - protein, 312 aa, synthetic construct
  VARIANT 155: note = X can be any amino acid
  REGION 1..312: note = DARPin Dimer Protein
DLGKKLLEAARAGQDDEVRILMANGADVNAADPAGWTPLHLAACYGHLEIVGVLLKNGADVNALDWGGWTPLHLAAVIGHLEIVGVLLKHGADVNATDLSGWTPLHLAAFRGHLEIVEVLLKHGADVNAQDKFGKTAFDISIDNGNEDLAEILQXDLGKKLLEAARAGQDDEVRILMANGADVNARDFPGLTPLHLAAVQGHLEIVDVLLKNGADVNAVDKLGLTPLHLAARRGHLEIVEVLLKHGADVNAVDYTGTTPLHLAAVPGHLEIVEVLLKNGADVNAQDKFGKTAFDISIDNGNEDLAEILQSSS

SEQ ID NO: 60 - protein, 312 aa, synthetic construct
  VARIANT 155: note = X can be any amino acid
  REGION 1..312: note = DARPin Dimer Protein
DLGKKLLEAARAGQDDEVRILMANGADVNAMDRHGQTPLHLAASFGHLEIVEVLLKHGADVNANDYSGGTPLHLAAIWGHLEIVGVLLKNGADVNAKDVWGRTPLHLAAIGGHLEIVEVLLKNGADVNAQDKFGKTAFDISIDNGNEDLAEILQXDLGKKLLEAARAGQDDEVRILMANGADVNARDFPGLTPLHLAAVQGHLEIVEVLLKNGADVNAVDKLGLTPLHLAARRGHLEIVEVLLKHGADVNAVDYTGTTPLHLAAVPGHLEIVEVLLKNGADVNAQDKFGKTAFDISIDNGNEDLAEILQSSS

SEQ ID NO: 61 - protein, 312 aa, synthetic construct
  VARIANT 155: note = X can be any amino acid
  REGION 1..312: note = DARPin Dimer Protein
DLGKKLLEAARAGQDDEVRILMANGADVNAGDPFGFTPLHLAAMFGHLEIVEVLLKNGADVNAHDEYGFTPLHLAAVVGHLEIVEVLLKNGADVNACDNQGGTPLHLAARIGHLEIVEVLLKHGADVNAQDKFGKTAFDISIDNGNEDLAEILQXDLGKKLLEAARAGQDDEVRILMANGADVNARDFPGLTPLHLAAVQGHLEIVDVLLKNGADVNAVDKLGLTPLHLAARRGHLEIVEVLLKHGADVNAVDYTGTTPLHLAAVPGHLEIVEVLLKNGADVNAQDKFGKTAFDISIDNGNEDLAEILQSSS

SEQ ID NO: 62 - protein, 312 aa, synthetic construct; identical to SEQ ID NO: 56
  VARIANT 155: note = X can be any amino acid
  REGION 1..312: note = DARPin Dimer Protein

SEQ ID NO: 63 - protein, 312 aa, synthetic construct
  VARIANT 155: note = X can be any amino acid
  REGION 1..312: note = DARPin Dimer Protein
DLGKKLLEAARAGQDDEVRILMANGADVNAADPAGWTPLHLAACYGHLEIVGVLLKNGADVNALDWGGWTPLHLAAVIGHLEIVGVLLKHGADVNATDLSGWTPLHLAAFRGHLEIVEVLLKHGADVNAQDKFGKTAFDISIDNGNEDLAEILQXDLGKKLLEAARAGQDDEVRILMANGADVNARDESGTTPLHLAAGSGHLEIVEVLLKHGADVNAADLGGWTPLHLAAIWGHLEIVEVLLKHGADVNATDPTGWTPLHLAALGGHLEIVEVLLKNGADVNAQDKFGKTAFDISIDNGNEDLAEILQSSS

SEQ ID NO: 64 - protein, 312 aa, synthetic construct; identical to SEQ ID NO: 56
  VARIANT 155: note = X can be any amino acid
  REGION 1..312: note = DARPin Dimer Protein

SEQ ID NO: 65 - protein, 312 aa, synthetic construct
  VARIANT 155: note = X can be any amino acid
  REGION 1..312: note = DARPin Dimer Protein
DLGKKLLEAARAGQDDEVRILMANGADVNARDFPGLTPLHLAAVQGHLEIVEVLLKNGADVNAVDKLGLTPLHLAARRGHLEIVEVLLKHGADVNAVDYTGTTPLHLAAVPGHLEIVEVLLKNGADVNAQDKFGKTAFDISIDNGNEDLAEILQXDLGKKLLEAARAGQDDEVRILMANGADVNAGDPFGFTPLHLAAMFGHLEIVEVLLKNGADVNAHDEYGFTPLHLAAVVGHLEIVEVLLKNGADVNACDNQGGTPLHLAAHTGHLEIVEVLLKHGADVNAQDKFGKTAFDISIDNGNEDLAEILQSSS

SEQ ID NO: 66 - protein, 312 aa, synthetic construct
  VARIANT 155: note = X can be any amino acid
  REGION 1..312: note = DARPin Dimer Protein
DLGKKLLEAARAGQDDEVRILMANGADVNAMDRHGQTPLHLAASFGRLEIVEVLLKHGADVNANDYSGGTPLHLAAIWGHLEIVGVLLKNGADVNAKDVWGRTPLHLAAIGGHLEIVEVLLKNGADVNAQDKFGKTAFDISIDNGNEDLAEILQXDLGKKLLEAARAGQDDEVRILMANGADVNARDFPGLTPLHLAAVQGHLEIVEVLLKNGADVNAVDKLGLTPLHLAARRGHLEIVEVLLKHGADVNAVDYTGTTPLHLAAVPGHLEIVEVLLKNGADVNAQDKFGKTAFDISIDNGNEDLAEILQSSS

SEQ ID NO: 67 - protein, 312 aa, synthetic construct; identical to SEQ ID NO: 65
  VARIANT 155: note = X can be any amino acid
  REGION 1..312: note = DARPin Dimer Protein

SEQ ID NO: 68 - protein, 312 aa, synthetic construct; identical to SEQ ID NO: 56
  VARIANT 155: note = X can be any amino acid
  REGION 1..312: note = DARPin Dimer Protein

SEQ ID NO: 69 - protein, 312 aa, synthetic construct; identical to SEQ ID NO: 52
  VARIANT 155: note = X can be any amino acid
  REGION 1..312: note = DARPin Dimer Protein

SEQ ID NO: 70 - protein, 312 aa, synthetic construct
  VARIANT 155: note = X can be any amino acid
  REGION 1..312: note = DARPin Dimer Protein
DLGKKLLEAARAGQDDEVRILMANGADVNARDFPGLTPLHLAAVQGHLEIVEVLLKNGADVNAVDKLGLTPLHLAARRGHLEIVEVLLKHGADVNAVDYTGTTPLHLAAVPGHLEIVEVLLKNGADVNAQDKFGKTAFDISIDNGNEDLAEILQXDLGKKLLEAARAGQDDEVRILMANGADVNAGDPFGFTPLHLAAMFGHLEIVEVLLKNAADVNAHDEYGFTPLHLAAVVGHLEIVEVLLKNGADVNACDNQGGTPLHLAARIGHLEIVEVLLKHGADVNAQDKFGKTAFDISIDNGNEDLAEILQSSS

SEQ ID NO: 71 - protein, 345 aa, synthetic construct
  VARIANT 188: note = X can be any amino acid
  REGION 1..345: note = DARPin Dimer Protein
DLGKKLLEAARAGQDDEVRILMANGADVNACDSNGRTPLHLAAVRGHLEIVEVLLKNGADVNAWDVIGKTPLHLAADGGHLEIVEVLLKNGADVNAWDLIGRTPLHLAALDGHLEIVEVLLEHGADVNAYDRPGHTPLHLAARRGHLEIVEVLLKYGADVNAQDKFGKTAFDISIDNGNEDLAEILQXDLGKKLLEAARAGQDDEVRILVANGADVNAGDPFGFTPLHLAALYGHLEIVEVLLKNGADVNAHEEYGFTPLHLAAVVSHMEIVEVLLNNGADVNARDNQGGSPLHLASHTGHLEIVEVLLKQGADVNAQDKFGKTAYDISIDIGNEDLAEILQSSS

SEQ ID NO: 72 - protein, 33 aa, synthetic construct
  REGION 1..33: note = tagging molecule
MGSSHHHHHHSSGLVPRGSHMEQKLISEEDLGS

SEQ ID NO: 73 - protein, 27 aa, synthetic construct
  REGION 1..27: note = linker
SSSGGGGSEFGGGGSGGGGSGGGGSAS

SEQ ID NO: 74 - protein, 319 aa, Escherichia coli
  REGION 1..319: note = Stx2a holotoxin
MKCILFKWVLCLLLGFSSVSYSREFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVRAVNEESQPECQITGDRPVIKINNTLWESNTAAAFLNRKSQFLYTTGK

SEQ ID NO: 75 - DNA, 14 bp, Escherichia coli
  source 1..14: mol_type = genomic DNA
  misc_feature 1..14: note = DNA sequence sandwiched between the A and B subunit
taaaggagttaagc

SEQ ID NO: 76 - protein, 97 aa, Escherichia coli
  REGION 1..97: note = Stx2a holotoxin
MKKMFMAVLFALASVNAMAADCAKGKIEFSKYNEDDTFTVKVDGKEYWTSRWNLQPLLQSAQLTGMTVTIKSSTCESGSGFAEVQFNNDLEHHHHHH

SEQ ID NO: 77 - protein, 292 aa, Escherichia coli
  REGION 1..292: note = Stx2a-A1
MKCILFKWVLCLLLGFSSVSYSREFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVRAVNEESQPEGGSLEHHHHHH

SEQ ID NO: 78 - protein, 95 aa, Escherichia coli
  REGION 1..95: note = Stx2a-B
MKKMFMAVLFALASVNAMAADCAKGKIEFSKYNEDDTFTVKVDGKEYWTSRWNLQPLLQSAQLTGMTVTIKSSTCESGSGFAEVQFNNDHHHHHH

SEQ ID NO: 79 - protein, 152 aa, synthetic construct
  REGION 1..152: note = DARPin 10B
GKKLLEAARAGQDDEVRILMANGADVNAGDPFGFTPLHLAAMFGHLEIVEVLLKNGADVNAHDEYGFTPLHLAAVVGHLEIVEVLLKNGADVNACDNQGGTPLHLAAVDGHLEIVEVLLKHGADVNAQDKFGKTAFDISIDNGNEDLAEILQ

SEQ ID NO: 80 - protein, 152 aa, synthetic construct
  REGION 1..152: note = DARPin HT
GKKLLEAARAGQDDEVRILMANGADVNAGDPFGFTPLHLAAMFGHLEIVEVLLKNGADVNAHDEYGFTPLHLAAVVGHLEIVEVLLKNGADVNACDNQGGTPLHLAAHTGHLEIVEVLLKHGADVNAQDKFGKTAFDISIDNGNEDLAEILQ

SEQ ID NO: 81 - protein, 152 aa, synthetic construct
  REGION 1..152: note = DARPin RI
GKKLLEAARAGQDDEVRILMANGADVNAGDPFGFTPLHLAAMFGHLEIVEVLLKNGADVNAHDEYGFTPLHLAAVVGHLEIVEVLLKNGADVNACDNQGGTPLHLAARIGHLEIVEVLLKHGADVNAQDKFGKTAFDISIDNGNEDLAEILQ

SEQ ID NO: 82 - protein, 152 aa, synthetic construct
  REGION 1..152: note = DARPin SHT
GKKLLEAARAGQDDEVRILVANGADVNAGDPFGFTPLHLAALYGHLEIVEVLLKNGADVNAHEEYGFTPLHLAAVVSHMEIVEVLLNNGADVNACDNQGGTPLHLASHTGHLEIVEVLLKQGADVNAQDKFGKTAYDISIDIGNEDLAEILQ

SEQ ID NO: 83 - protein, 152 aa, synthetic construct
  VARIANT 29: note = X can be any amino acid
  VARIANT 31..32: note = X can be any amino acid
  VARIANT 34: note = X can be any amino acid
  VARIANT 42..43: note = X can be any amino acid
  VARIANT 55: note = X can be any amino acid
  VARIANT 62: note = X can be any amino acid
  VARIANT 64..65: note = X can be any amino acid
  VARIANT 67: note = X can be any amino acid
  VARIANT 75..76: note = X can be any amino acid
  VARIANT 88: note = X can be any amino acid
  VARIANT 95: note = X can be any amino acid
  VARIANT 97..98: note = X can be any amino acid
  VARIANT 100: note = X can be any amino acid
  VARIANT 108..109: note = X can be any amino acid
  VARIANT 121: note = X can be any amino acid
GKKLLEAARAGQDDEVRILMANGADVNAXDXXGXTPLHLAAXXGHLEIVEVLLKXGADVNAXDXXGXTPLHLAAXXGHLEIVEVLLKXGADVNAXDXXGXTPLHLAAXXGHLEIVEVLLKXGADVNAQDKFGKTAFDISIDNGNEDLAEILQ

SEQ ID NO: 84 - protein, 319 aa, Escherichia coli
  REGION 1..319: note = Stx2c-A
MKCILFKWVLCLLLGFSSVSYSREFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVRAVNEDSQPECQITGDRPVIKINNTLWESNTAAAFLNRKSQFLYTTGK

SEQ ID NO: 85 - protein, 89 aa, Escherichia coli
  REGION 1..89: note = Stx2a-B
MKKMFMAVLFALASVNAMAADCAKGKIEFSKYNEDDTFTVKVDGKEYWTSRWNLQPLLQSAQLTGMTVTIKSSTCESGSGFAEVQFNND

SEQ ID NO: 86 - protein, 89 aa, Escherichia coli
  REGION 1..89: note = Stx2c-B
MKKMFMAVLFALVSVNAMAADCAKGKIEFSKYNENDTFTVKVAGKEYWTSRWNLQPLLQSAQLTGMTVTIKSSTCESGSGFAEVQFNND

(19) United States
(12) Patent Application Publication
(10) Pub. No.: US 2026/0085097 A1
(43) Pub. Date: Mar. 26, 2026

(54) DARPIN-CONTAINING COMPOSITIONS AND METHODS THEREOF

(71) Applicant: THE TEXAS A&M UNIVERSITY SYSTEM, College Station, TX (US)

(72) Inventors: Zhilei CHEN, College Station, TX (US); Yu ZENG, College Station, TX (US); Zeyu PENG, College Station, TX (US); Rudo SIMEON, College Station, TX (US)

(21) Appl. No.: 19/112,736

(22) PCT Filed: Sep. 20, 2023
(86) PCT No.: PCT/US2023/033238

§ 371 Date: Mar. 18, 2025

Related U.S. Application Data
(60) Provisional application No. 63/408,247, filed on Sep. 20, 2022.

(51) Int. Cl. C07K 14/245; A61K 38/00; A61P 31/04

(52) U.S. Cl. CPC C07K 14/245; A61P 31/04; A61K 38/00; C07K 2318/20

(57) ABSTRACT

The present disclosure provides compositions comprising a designed ankyrin repeat protein (DARPin) for use in treating Shiga toxin (Stx)-producing Escherichia coli (STEC) and related diseases, including hemolytic uremic syndrome (HUS). The high thermostability and high microbial expression yield make DARPin an attractive alternative to antibody therapeutics. The present disclosure provides monomer proteins, dimer proteins, and trimer proteins, as well as pharmaceutical compositions and methods utilizing the same.

### CROSS-REFERENCE TO RELATED APPLICATIONS

This application claims the benefit under 35 USC § 119 (e) of U.S. Provisional Application Ser. No. 63/408,247, filed on Sep. 20, 2022, the entire disclosure of which is incorporated herein by reference.

### GOVERNMENT SUPPORT

This invention was made with government support under R21AI137803 awarded by the National Institutes of Health. The government has certain rights in the invention.

### REFERENCE TO SEQUENCE LISTING SUBMITTED ELECTRONICALLY

The official copy of the sequence listing is submitted electronically via EFS-Web as an XML formatted sequence listing with a file named 78090-395139_SL.xml, created on Sep. 19, 2023 and having a size of 134.2 kilobytes and is filed concurrently with the specification. The sequence listing comprised in this XML formatted document is part of the specification and is herein incorporated by reference in its entirety.

### BACKGROUND AND SUMMARY OF THE INVENTION

Shiga toxin (Stx)-producing Escherichia coli (STEC) is one of the most common infectious causes of bloody diarrhea, also known as hemorrhagic colitis (HC). It is estimated that 265,000 STEC infections occur in the United States each year. For instance, infection can occur when patients ingest contaminated food (e.g. salad and under cooked meats) or water (e.g. drinking water or swimming pool water), or by direct contact with infected animals or humans. Although GI dysfunction is the most visible symptom of the infection, a serious complication of STEC infection can be hemolytic uremic syndrome (HUS).

HUS is characterized by acute renal failure, thrombocytopenia and microangiopathic hemolytic anemia and occurs in 4-15% of STEC infection cases, for instance in children and the elderly. About 40% of the patients with HUS develop chronic complications. The largest documented outbreak of STEC infection worldwide occurred in Germany in 2011, affecting 3816 patients and causing 54 deaths. Among these, 845 patients developed HUS.

Currently, there is no effective treatment for STEC infection. The pathology of STEC infection is primarily caused by the action of two exotoxins (i.e., Shiga toxin 1 (Stx1) and Shiga toxin 2 (Stx2)) that attack the ribosomes and trigger apoptosis upon their uptake by the host cells. Intimin, an adhesin molecule, is another important virulence factor which facilitates bacterial attachment to colonocytes. Although antibiotic treatment can reduce the STEC load, it also increases Stx-phage synthesis and Shiga toxin release, leading to an increased risk of developing HUS and death. Consequently, CDC guidelines recommend that persons infected with STEC be provided with only supportive cares such as oral and IV fluids and pain control and not be treated with antibiotics. Five predominant Stx1 subtypes and ten Stx2 subtypes have been reported, with Stx2a, Stx1a, and Stx2c mainly associated with HUS. Stx2 exhibits higher toxicity than Stx1 in both mice and humans.

Shiga toxins belong to the AB₅ family of protein toxins and are comprised of an enzymatically active A subunit and a non-toxic pentameric B subunit. Interaction between the B subunit and the carbohydrate moiety of the glycosphingolipid Gb₃ on the extracellular leaflet of cell plasma membranes enables these toxins to enter host cells through receptor-mediated endocytosis and be retrogradely transported from the Golgi apparatus to the endoplasmic reticulum (ER). Some Shiga toxins are able to cross the epithelial cell barrier and enter the circulatory systems, from where they travel to the kidneys and damage Gb₃-expressing glomerular endothelial cells, causing the onset of HUS.

In recent years, a number of Shiga toxin-neutralizing antibodies have been developed as therapeutics for STEC. However, complicated logistics and excessive costs have prevented a full clinical evaluation of these anti-toxin antibodies. For instance, antibody production requires mammalian cell culture which is expensive to maintain. Further, the sporadic nature of STEC infection, combined with the limited shelf-life and high cost of antibodies, can slow development of antibody-based STEC therapeutics and also lower the need to produce and stockpile anti-toxin antibodies. Therefore, there exists a need for new compositions and methods to treat STEC-related disease and its associated complications.

Accordingly, the present disclosure provides compositions comprising a designed ankyrin repeat protein (DARPin) for use in treating STEC and related diseases. DARPin is a small scaffold protein (˜18 kDa) can be engineered to bind diverse targets and enjoys a low immunogenicity potential, a very high expression yield in microorganisms (15 g per liter of E. coli culture in fermenters or 23% of dried cell weight), high solubility and long shelf life. The high thermostability and high microbial expression yield make DARPin an attractive alternative to antibody therapeutics.

The present disclosure provides a protein engineering approach that utilizes directed evolution and rational design. The resultant compositions can provide potent neutralization activity against Stx2a. In addition, the result compositions can target the toxin A-subunit and neutralizes the toxin catalytic activity as well as binding the B-subunit. Furthermore, the resulting compositions are believed to induce a novel conformational change in the B-subunit that distorts its five-fold symmetry and interferes with toxin attachment. As described herein, the resultant compositions can be developed for treatment of STEC infection, including prevention of HUS complications.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows an image of an SDS-PAGE (4-15%) of the purified proteins under reducing conditions and their protein sequences.

FIG. 2 shows a representative graph of the toxicity of holotoxin Stx2a (SEQ ID NO: 74-78). Vero E6 cells (1.5×10³ cells/well) were mixed with the appropriately diluted Stx2a and the mixture was added to 96-well plate. The cell viability was quantified 3 days later using the CellTiterGlo assay.

FIG. 3 shows a scheme of enrichment of STX2-binding DARPins after phage panning. Successive rounds of a DARPin-phage library were panned against STX2 using streptavidin-coated magnetic beads coated with 100 nM biotinylated-STX2 and MaxiSorp 96-well plates coated with neutravidin (66 nM) and 100 nM biotin-STX2 in alternative rounds. The binding of phage recovered from successive rounds of panning to STX2 was quantified using ELISA. Significant binding was observed after three rounds of panning.

FIG. 4A shows a representative graph of the characterization of parental anti-Stx2 DARPins discovered from a phage-displayed naïve DARPin library. Relative viability is shown at varying concentrations of DARPin. Monomeric DARPins protected Vero E6 cells from Stx2a-induced cytopathic effect. Each well contains 1.5×10³ Vero cells, 5 μg/mL (or 57 fM) Stx2a and the appropriately diluted DARPin. Error bars represent the standard deviation of two independent experiments performed in duplicate.

FIG. 4B shows a representative graph of the characterization of parental anti-Stx2 DARPins discovered from a phage-displayed naïve DARPin library. Relative binding of monomeric DARPins to Stx2a determined using ELISA. Error bars represent the standard deviation of two independent experiments.

FIG. 5A shows a representative graph of ELISA-based assay to evaluate the ability of monomeric DARPin 10B to bind the A- and B-subunits of Stx2a. The error bars represent the mean deviation of duplicate samples from one representative experiment.

FIG. 5B shows a representative graph of ELISA-based assay to evaluate the ability of monomeric DARPin #20 to bind the A- and B-subunits of Stx2a. The error bars represent the mean deviation of duplicate samples from one representative experiment.

FIG. 5C shows a representative graph of ELISA-based assay to evaluate the ability of monomeric DARPin #3 to bind the A- and B-subunits of Stx2a. The error bars represent the mean deviation of duplicate samples from one representative experiment.

FIG. 5D shows a representative graph of ELISA-based assay to evaluate the ability of monomeric DARPin #16 to bind the A- and B-subunits of Stx2a. The error bars represent the mean deviation of duplicate samples from one representative experiment.

FIG. 6A shows an illustrative schematic of affinity maturation strategy.

FIG. 6B shows the sequence of DARPin 10B (SEQ ID NO: 79). The red residues were subjected to saturation mutagenesis.

FIG. 6C shows homology model of DARPin 10B. Both green and red residues were randomized in the original DARPin library. The red residues were subjected to saturation mutagenesis.

FIG. 7A shows a representative graph of relative viability at varying concentrations of DARPin HT, RI and 10B. The ELISA plates were coated with the full-length Stx2a.

FIG. 7B shows a representative graph of binding ability at varying concentrations of DARPin HT, RI and 10B. The ELISA plates were coated with the full-length Stx2a. (C) Amino acid sequences of DARPin HT and RI.

FIG. 7C shows amino acid sequences of DARPin HT (SEQ ID NO: 80) and RI (SEQ ID NO: 81).

FIG. 8 shows a representative graph of activity comparison of DARPin dimers.

FIG. 9A shows a homology model of DARPin SHT showing derivation of key putative Stx2a-binding residues. The green, blue and red residues are derived from the original DARPin library, error-prone PCR library and saturation mutagenesis library. (C) Protection of Vero E6 cells by the different DARPins. Vero E6 cells (1.5×10³ cells/well) were incubated with 5 μg/mL (57 fM) Stx2a and the appropriately diluted DARPin for 3 days. Error bars represent the standard deviation of two independent experiments performed in duplicate. (D) Summary of EC₅₀ values.

FIG. 9B shows an amino acid sequence of DARPin SHT (SEQ ID NO: 82) showing derivation of key putative Stx2a-binding residues. The green, blue and red residues are derived from the original DARPin library, error-prone PCR library and saturation mutagenesis library.

FIG. 9C shows a representative graph of protection of Vero E6 cells by the different DARPins. Vero E6 cells (1.5×10³ cells/well) were incubated with 5 μg/mL (57 fM) Stx2a and the appropriately diluted DARPin for 3 days. Error bars represent the standard deviation of two independent experiments performed in duplicate.

FIG. 9D shows a table of the summary of EC₅₀ values of the different DARPins determined from FIG. 9C.

FIG. 10 shows a table of sequence alignment of off-rate mutants with respect to SEQ ID NO: 80, 81, and 83.

FIG. 11A shows a sequence alignment of Stx2a (SEQ ID NO: 74 and 85) and Stx2c (SEQ ID NO: 84 and 86).

FIG. 11B shows a representative graph of protection of Vero cells by SD5. Vero E6 cells (1.5×10³ cells/well) were incubated with 60 μg/mL Stx2c and the appropriately diluted DARPin for 3 days. Error bars represent the standard deviation of two independent experiments performed in duplicate.

FIG. 12 shows a representative graph showing DARPin molecules may protect ribosomes from intoxication by Stx2a-A1 in vitro. Purified Stx2a-A1 (2.5 μM) was incubated with DARPin molecules (final 10 μM) in PBS supplemented with BSA (40 mg/mL) for 30 minutes at 37° C. before being added to 1×NEBExpress Cell-free E. coli protein synthesis system which contains both the translation and transcription machinery. The mixture was incubated at 37° C. for 30 minutes before the addition of a reporter plasmid encoding GFP, and the protein synthesis was continued at 37° C. for 6 hours followed by overnight incubation at 4° C. to ensure complete folding of GFP. The amount of GFP produced was quantified using a fluorescent plate reader and compared to that from reactions lacking any toxin (positive control, PC) and reactions with toxin but without any DARPin (negative control, NC).

FIG. 13A shows a representative graph of the relative binding of DARPins to Stx2a-A1 fragment determined by ELISA. DARPins were added to MaxiSorp plates coated with 4 μg/mL Stx2a-A1. A representative experiment with the mean of 2 technical replicates is shown.

FIG. 13B shows a summary table of the calculated EC₅₀ values determined from FIG. 13A.

FIG. 14A shows a representative graph of the binding kinetics of selected DARPin 10B. A sensorgram is shown of DARPin 10B under equilibrium binding condition. (F) Sensorgram of a sequential binding experiment in which the biosensor was first loaded with DARPin SHT (125 nM) for 120 s to reach saturation. The sensor was then dipped into buffer containing DARPin #3 (125 nM) for 30 s to record the binding kinetics. (G) Summary of the binding and rate constants.

FIG. 14B shows a representative graph of the binding kinetics of selected DARPin HT. A sensorgram is shown of DARPin HT under equilibrium binding condition.

FIG. 14C shows a representative graph of the binding kinetics of selected DARPin SHT. A sensorgram is shown of DARPin SHT under equilibrium binding condition.

FIG. 14D shows a representative graph of the binding kinetics of selected DARPin #3. A sensorgram is shown of DARPin #3 under equilibrium binding condition.

FIG. 14E shows a representative graph of the binding kinetics of selected DARPin SD5. A sensorgram is shown of DARPin SD5 under equilibrium binding condition.

FIG. 14F shows a sensorgram of a sequential binding experiment in which the biosensor was first loaded with DARPin SHT (125 nM) for 120 s to reach saturation. The sensor was then dipped into buffer containing DARPin #3 (125 nM) for 30 s to record the binding kinetics.

FIG. 14G shows a summary table of the binding and rate constants determined in FIGS. 14A-14F.

FIG. 15A shows an illustrative schematic of DARPin DA1-SD5 in comparison to SD5.

FIG. 15B shows a representative graph of evaluation of the in vivo activity. DARPin DA1-SD5 retains similar toxin-neutralization potency as SD5 as determined by Vero E6 cell viability assay.

FIG. 15C shows a representative graph of evaluation of the in vivo activity. DARPin DA1-SD5 retains similar toxin-neutralization potency as SD5 as determined by Vero E6 cell viability assay. DARPin DA1-SD5 protected mice from Stx2a toxicity (n=5). Mantel-Cox test, *p<0.002.

FIG. 15D shows a representative graph of evaluation of the in vivo activity. DARPin DA1-SD5 retains similar toxin-neutralization potency as SD5 as determined by Vero E6 cell viability assay. Toxin neutralization activity DA1-SD5 after storage at room temperature for 1-4 weeks as determined by Vero E6 cell viability assay.

FIG. 16A shows a representative micrograph of the cryo-EM data of cryo-EM analysis on Stx2a and DARPin #3 complex.

FIG. 16B shows a representative 2D classification of the complex from analysis on Stx2a and DARPin #3 complex. The white arrows highlight the extra density of DARPin.

FIG. 16C shows a representative graph of Fourier Shell Correlation (FSC) of the bound-state map and apo-state map from analysis on Stx2a and DARPin #3 complex.

FIG. 16D shows illustrative density maps of the bound state (left) and the apo state (right) from analysis on Stx2a and DARPin #3 complex. with the refined models docked in. The A subunit is colored red. The B subunit is colored blue. The DARPin is colored orange.

FIG. 17 shows a representative schematic of data processing for the complex between Stx2a and DARPin #3.

FIG. 18A shows a side view image of the cryo-EM structure of the Stx2a-DARPin #3 complex. A subunit is colored red. The individual monomers in the B subunit are colored in different shades of blue. DARPin #3 is colored orange.

FIG. 18B shows a zoom-in view image of the cryo-EM structure of the Stx2a-DARPin #3 complex. The bound modeled Gb₃ molecules are colored magenta.

FIG. 18C shows a bottom view image of the cryo-EM structure of the Stx2a-DARPin #3 complex. The bound modeled Gb₃ molecules are colored magenta.

FIG. 18D shows a schematic analysis of B subunit rotation in the apo and bound states on Stx2a. The vectors represent the relative displacement between the equivalent Ca atoms in the B subunit in the apo and DARPin-bound state with large and small movements indicated by red and blue colors, respectively.

### DETAILED DESCRIPTION

Various embodiments of the invention are described herein as follows. In particular, following sequences are described herein and included in the various aspects of the disclosure:

DARPin Monomer Proteins

| | |
|---|---|
| DARPin | DLGKKLLEAARAGQDDEVRILMANGADVNARDESGTTPLHLAAGSG |
| #2 | HLEIVEVLLKHGADVNAADLGGWTPLHLAAIWGHLEIVEVLLKHGA |
| | DVNATDPTGWTPLHLAALGGHLEIVEVLLKNGADVNAQDKFGKTAF |
| | DISIDNGNEDLAEILQSSS (SEQ ID NO: 1) |
| DARPin | DLGKKLLEAARAGQDDEVRILMANGADVNACDSNGRTPLHLAAVR |
| #3 | GHLEIVEVLLKNGADVNAWDVIGKTPLHLAADGGHLEIVEVLLKNG |
| | ADVNAWDLIGRTPLHLAALDGHLEIVEVLLEHGADVNAYDRPGHTPL |
| | HLAARRGHLEIVEVLLKYGADVNAQDKFGKTAFDISIDNGNEDLAEIL |
| | QSSS (SEQ ID NO: 2) |
| DARPin | DLGKKLLEAARAGQDDEVRILMANGADVNAPDEYGSTPLHLAASLG |
| #4 | HLEIVEVLLKHGADVNATDRLGNTPLHLAALGGHLEIVEVLLKYGAD |
| | VNAADPAGWTPLHLAALGGHLEIVEVLLKYGADVNAQDKFGKTAFD |
| | ISIDNGNEDLAEILQSSS (SEQ ID NO: 3) |
| DARPin | DLGKKLLEAARAGQDDEVRILMANGADVNACDAGGTTPLHLAASG |
| #5 | GHLEIVEVLLKNGADVNAYDPGGWTPLHLAAIRGHLEIVEVLLKYGA |
| | DVNAEDPLGWTPLHLAALAGHLEIVEVLLKNGADVNAQDKFGKTAF |
| | DISIDNGNEDLAEILQSSS (SEQ ID NO: 4) |
| DARPin | DLGKKLLEAARAGQDDEVRILMANGADVNAGDPFGFTPLHLAAMFG |
| #6 | HLEIVEVLLKNGADVNAHDEYGFTPLHLAAVVGHLEIVEVLLKNGAD |
| | VNACDNQGGTPLHLAAVDGHLEIVEVLLKHGADVNAQDKFGKTAFD |
| | ISIDNGNEDLAEILQSSS (SEQ ID NO: 5) |
| DARPin | DLGKKLLEAARAGQDDEVRILMANGADVNASDSVGWTPLHLAADR |
| #7 | GHLEIVEVLLKNGADVNAYDPGGWTPLHLAAIRGHLEIVEVLLKYGA |
| | DVNAEDPLGWTPLHLAALAGHLEIVEVLLKNGADVNAQDKFGKTAF |
| | DISIDNGNEDLAEILQSSS (SEQ ID NO: 6) |
| DARPin | DLGKKLLEAARAGQDDEVRILMANGADVNAMDRHGQTPLHLAASF |
| #8 | GHLEIVEVLLKHGADVNANDYSGGTPLHLAAIWGHLEIVGVLLKNGA |
| | DVNAKDVWGRTPLHLAAIGGHLEIVEVLLKNGADVNAQDKFGKTAF |
| | DISIDNGNEDLAEILQSSS (SEQ ID NO: 7) |
| DARPin | DLGKKLLEAARAGQDDEVRILMANGADVNARDHLGHTPLHLAASRG |
| #9 | HLEIVEVLLKHGADVNASDSVGQTPLHLAAVWGHLEIVEVLLKNGA |
| | DVNALDTLGWTPLHLAALGGHLEIVEVLLKHGADVNAQDKFGKTAF |
| | DISIDNGNEDLAEILQSSS (SEQ ID NO: 8) |
| DARPin | DLGKKLLEAARAGQDDEVRILMANGADVNARDFPGLTPLHLAAVQG |
| #10 | HLEIVEVLLKNGADVNAVDKLGLTPLHLAARRGHLEIVEVLLKHGAD |
| | VNAVDYTGTTPLNLAAVPGHLEIVEVLLKNGADVNAQDKFGKTAFDI |
| | SIDNGNEDLAEILQSSS (SEQ ID NO: 9 |
| DARPin | DLGKKLLEAARAGQDDEVRILMANGADVNAMDRHGQTPLHLAASF |
| #11 | GRLEIVEVLLKHGADVNANDYSGGTPLHLAAIWGHLEIVGVLLKNGA |
| | DVNAKDVWGRTPLHLAAIGGHLEIVEVLLKNGADVNAQDKFGKTAF |
| | DISIDNGNEDLAEILQSSS (SEQ ID NO: 10 |
| DARPin | DLGKKLLEAARAGQDDEVRILMANGADVNASDSVGWTPLHLAADR |
| #12 | GHLEIVEVLLKNGADVNAYDPGGWTPLHLAAIRGHLEIVEVLLKYGA |
| | DVNAEDPLGWTPLHLAALAGHLEIVEVLLRNGADVNAQDKFGKTAF |
| | DISIDNGNEDLAEILQSSS (SEQ ID NO: 11 |
| DARPin | DLGKKLLEAARAGQDDEVRILMANGADVNARDFPGLTPLHLAAVQG |
| #13 | HLEIVEVLLKNGADVNAVDKLGLTPLHLAARRGHLEIVEVLLKHGAD |
| | VNAVDYTGTTPLHLAAVPGHLEIVEVLLKNGADVNAQDKFGKTAFDI |
| | SIDNGNEDLAEILQSSS (SEQ ID NO: 12 |
| DARPin | DLGKKLLEAARAGQDDEVRILVANGADVNAPDEYGSTPLHLAASLG |
| #14 | HLEIVEVLLKHGADVNATDRLGNTPLHLAALGGHLEIVEVLLKYGAD |
| | VNAADPAGWTPLHLAALGGHLEIVEVLLKYGADVNAQDKFGKTAFD |
| | ISIDNGNEDLAEILQSSS (SEQ ID NO: 13 |
| DARPin | DLGKKLLEAARAGQDDEVRILMANGADVNAKDRHGQTPLHLAASFG |
| #15 | HLEIVEVLLKHGADVNANDYSGGTPLHLAAIWGHLEIVGVLLKNGAD |
| | VNAKDVWGRTPLHLAAIGGHLEIVEVLLKNGADVNAQDKFGKTAFD |
| | ISIDNGNEDLAEILQSSS (SEQ ID NO: 14 |
| DARPin | DLGKKLLEAARAGQDDEVRILMANGADVNAADPAGWTPLHLAACY |
| #16 | GHLEIVGVLLKNGADVNALDWGGWTPLHLAAVIGHLEIVGVLLKHG |
| | ADVNATDLSGWTPLHLAAFRGHLEIVEVLLKHGADVNAQDKFGKTA |
| | FDISIDNGNEDLAEILQSSS (SEQ ID NO: 15 |
| DARPin | DLGKKLLVAARAGQDGEVRILMANGADVNASDSVGWTPLHLAADR |
| #17 | GHLEIVEVLLKNGADVNAYDPGGWTPLHLAAIRGHLEIVEVLLKYGA |
| | DVNAEDPLGWTPLHLAALAGHLEIVEVLLKNGADVNAQDKFGKTAF |
| | DISIDNGNEDLAEILQSSS (SEQ ID NO: 16) |
| DARPin | DLGKKLLEAARAGQDDEVRILMANGADVNAGDPFGFTPLHLAAMFG |
| Off-rate | HLEIVEVLLKNGADVNAHEEYGFTPLHLAAVVGHLEIVEVLLNNGAD |
| #1 | VNACDNQGGTPLHLAAHTGHLEIVEVLLKHGADVNAQDKFGKTAFD |
| | ISIDIGNEDLAEILQSSS (SEQ ID NO: 17) |
| DARPin | DLGKKLLVAARAGQDDEVRILMANGADVNAGDPFGFTPLHLAAMFG |
| Off-rate | HLEIVEVLLKNGADVNAHEEYGFTPLHLAAVVGHLEIVEVLLKNGAD |
| #2 | VNACDNQGGTPLHLAAHTGHLEIVEVLLKHGADVNAQDKFGKTAFD |
| | ISIDNGNEDLAEILQSSS (SEQ ID NO: 18) |
| DARPin | DLGKKLLEAARAGQDDEVRILMANGADVNAGDPFGFTPLHLAAMFG |
| Off-rate | HLEIVEVLLKNGADVNAHEEYGFTPLHLAAVVSHMEIVEVLLKNGAD |
| #3 | VNACDNQGGTPLHLAAHTGHLEIVEVLLKHGADVNAQDKFGKTAFD |
| | ISIDNGNEDLAEILQSSS (SEQ ID NO: 19) |
| DARPin | DLGKKLLEAARAGQDDEVRILMANGADVNAGDPFGFTPLHLAAMFG |
| Off-rate | HLEIVEVLLKNGADVNAHEEYGFTPLHLAAVVGHLEIVEVLLKNGAD |
| #4 | VNACDNQGGTPLHLAARMGHLEMVEVLLKHGADVNAQDKFGKTAF |
| | DISIDNGNEDLAEILQSSS (SEQ ID NO: 20) |
| DARPin | DLGKKLLEAARAGQDDEVRILMANGADVNAGDPFGFTPLHLAAMFG |
| Off-rate | HLEIVEVLLKNGADVNAHEEYGFTPLHLAAVVGHLEIVEVLLKNGAD |
| #5 | VNACDNQGGTPLHLAARIGHLEIVEVLLKHGADVNAQDKFGKTAFDI |
| | SIDNGNEDLAEILQSSS (SEQ ID NO: 21) |
| DARPin | DLGKKLLEAARAGQDDEVRILVANGADVNAGDPFGFTPLHLAAMFG |
| Off-rate | HLEIVEVLLKNGADVNAHEEYGFTPLHLAAVVGHLEIVEVLLRNGAD |
| #6 | VNACDNQGGTPLHLAAHTGHLEIVEVLLKHGADVNAQDKFGKTAFD |
| | ISIDNGNEDLAEILQSSS (SEQ ID NO: 22) |
| DARPin | DLGKKLLEAARAGQDDEVRILMANGADVNAGDPFGFTPLHLAAMFG |
| Off-rate | HLEIVEVLLKNGADVNAHEEYGFTPLHLAAVVGHLEIVEVLLKNGAD |
| #8 | VNACDNQGGTPLHLAAHTGHLEIVEVLLKHGADVNAQDKFGKTAFD |
| | ISIDNGNEDLAEILQSSS (SEQ ID NO: 23) |
| DARPin | DLGKKLLEAARAGQDDEVRILMANGADVNAGDPFGFTPLHLAAMFG |
| Off-rate | HLEIVEVLLKNGADVNAHEEYGFTPLHLAAVVGLLEIVEVLLKNGAD |
| #10 | VNACDNQGGTPLHLAAHTGHLEIVEVLLKHGADVNAQDKFGKTAFD |
| | ISIGYGNEDLAEILQSSS (SEQ ID NO: 24) |
| DARPin | DLGKKLLEAARAGQDDEVRILMANGADVNVGDPFGFTPLHLAAMFG |
| Off-rate | HLEIVEVLLKNGADVNAHEEYGFTPLHLAAVVGHLEIVEVLLKNGAD |
| #11 | VNACDNQGGTPLHLAAHTGHLEIVEVLLKHGADVNAQDKFGKTAFD |
| | ISIDNGNEDLAEILQSSS (SEQ ID NO: 25) |
| DARPin | DLGKKLLEAARAGQDDEVRILMANGADVNAGDPFGFTPLHLAALYG |
| Off-rate | HLEIVEVLLKNGADVNAHEEYGFTPLHLAAVVGHLEIVEVLLKNGAD |
| #12 | VNACDNQGGTPLHLAAHTGHLEIVEVLLKHGADVNAQDKFGKTAFD |
| | ISIDNGNEDLAEILQSSS (SEQ ID NO: 26) |
| DARPin | DLGKKLLEAARAGQDDEVRILMANGADVNAGDPFGFTPLHLAAMY |
| Off-rate | GHLEIVEVLLKNGADVNAHEEYGFTPLHLAAVVGHLEIVEVLLKNGA |
| #13 | DVNACDNQGGTPLHLAAHTGHLEIVEVLLKHGADVNAQDKFGKTAY |
| | DISIDNGNEDLAEILQSSS (SEQ ID NO: 27) |
| DARPin | DLGKKLLEAARAGQDDVVRILMANGADVNAGDPFGFTPLHLAAMFG |
| Off-rate | HLEIAEVLSKNGADVNAHEEYGFTPLHLAAVVGHLGIVEVLLKNGAD |
| #14 | VNACDNQGGTPLHLAARIGHLEIVEVLLKHGADVNAQDKFGKTAFDI |
| | SIDNGNEDLAEILQSSS (SEQ ID NO: 28) |
| DARPin | DLGKKLLDAARTGQDDEVRILMANGADVNAGDPFGFTPLHLAALFG |
| Off-rate | HLEIVEVLLKNGADVNAHEEYGFTPLHLAAVVGHLEIVEVLLKNGAD |
| #16 | VNACDNQGGTPLHLAARIGHLEIVEVLLKQGADVNAQDKFGKTAFDI |
| | SIDNGNEDLAEILQSSS (SEQ ID NO: 29) |
| DARPin | DLGKKLLEAARAGQDDEVRILMANGADVNAGDPFGFTPLHLAAMFG |
| Off-rate | HLEIVEVLLMNGADVNAHEEYGFTPLHLAAVVGHLEIVGVLLKNGA |
| #17 | DVNASDNQGGTPLHLAARIGHLEIVEVLLKHGADVNAQDKFGKTAF |
| | DISIDNGNEDLAEILQSSS (SEQ ID NO: 30) |
| DARPin | DLGKKLLEAARAGQDDEVRILMANGADVNAGDPFGFTPLHLAAMFG |
| Off-rate | HLGIVEVLLKNGADVNAHEEYGFTPLHLAAVVGHLEIVEVLLKNGAD |
| #18 | VNACDNQGGTPLHLAARIGHLEIVEVLLKHGADVNAQDKFGKTAFDI |
| | SIDIGNEDLAEILQSSS (SEQ ID NO: 31) |
| DARPin | DLGKKLLEAARAGQDDEVRILMANGADVNAGDPFGFTPLHFAAMFG |
| Off-rate | HLEIVEVLLKNGADVNAHEEYGFTPLHLAAVVGHLEIVEVLLKNGAD |
| #19 | VNACDNQGGTPLHLAAHTGHLEIVEVLLKHGADVNAQDKFGKTAFD |
| | ISIDIGNEDLAEILQSSS (SEQ ID NO: 32) |
| DARPin | DLGKKLLEAARAGQDDEVRTLMANGADVNAGDPFGFTPLHLAAMY |
| Off-rate | GHLEIVEVLLKNGADVNAHEEYGFTPLHLAAVVGHLEIVEVLLKNGA |
| #20 | DVNACDNQGGTPLHLAAHTGHLEIVEVLLKHGADVNAQDKFGKTAF |
| | DISIDNGNEDLAEILQSSS (SEQ ID NO: 33) |
| DARPin | DLGKKLLEAARAGQDDEVRILMANGADVNAGDPFGFTPLHLAAMFG |
| Off-rate | HLEIVEVLLKNGAGVNAHEEYGFTPLHLAAVVGHLEIVEVLLKNGAD |
| #22 | VNACDNQGGTPLHLAAHTGHLEIVEVLLKHGADVNAQDKFGKTAFD |
| | ISIDNGNEDLAEILQSSS (SEQ ID NO: 34) |
| DARPin | DLGKKLLEAARAGQDDEVRILMANGADVNACDPFGFTPLHLAAMFG |
| Off-rate | HLEIVEVLLKNGADVNAHEEYGFTPLHLAAVVGHLEILEVLLKNGAD |
| #23 | VNARDNQGGSPLHLAAHTGHLEIVEVLLKRGADVNAQDKFGKTAFD |
| | ISIDNGNEDLAEILQSSS (SEQ ID NO: 35) |
| DARPin | DLGKKLLEAARAGQDDEVRILMANGADVNAGDPFGFTPLHLAAMFG |
| Off-rate | HLEIVEVLLKNGADVNAHEEYGFTPLHLAAVVGHLEIVEVLLNNGAD |
| #24 | VNACDNQGGTPLHLAAHTGHLEIVEVLLKHGADVNAQDKFGKTAFD |
| | ISIDNGNEDLAEILQSSS (SEQ ID NO: 36) |
| DARPin | DLGKKLLEAARAGQDDEVRILMANGADVNAGDPFGFTPLHLAAMFG |
| Off-rate | HLEIVEVLMKNGADANAHEEYGFTPLHLAAVVGHLEIVEVLLKNGA |
| #25 | DVNACDNQGGTPLHLAAHTGHLEIVEVLLKHGADVNAQDKFGKTAF |
| | DISIDNGNEDLAEILQSSS (SEQ ID NO: 37) |
| DARPin | DLGKKLLEAARAGQDDEVRILMANGADVNAGDPFGFTPLHLAAMFG |
| Off-rate | HLEIVEVLLKNGADVNAHEEYGFTPLHLAAVVGHLEIVEVLLKNGAD |
| #26 | VNACDNQGGTPLHLASHTGHLEIVEVLLKHGADVNAQDKFGKTAFDI |
| | SIDTGNEDLAEILQSSS (SEQ ID NO: 38) |
| DARPin | DLGKKLLEAARAGQDDEVRFLMANGADVNAGDPFGFTPLHLAAMF |
| Off-rate | GHLEIVEVLLKNGADENAHEEYGFTPLHLAAVVGHLEIVEVLLKNGA |
| #27 | DVNACDNQGGTPLHLAAHTGHLEIVEVLLKHGADVNAQDKFGKTAF |
| | DISIDNGNEDLAEILQSSS (SEQ ID NO: 39) |
| DARPin | DLGKKLLEAARAGQDDEVRILMANGADVNAGDPFGFTPLHLAAMY |
| Off-rate | GHLEIVEVLLKNGADVNAHEEYGFTPLHLAAVVGHLEIVEVLLKNGA |
| #28 | DVNACDNQGGTPLHLAARIGHLEIVEVLLKHGADVNAQDKFGKTAF |
| | DISIDNGNEDLAEILQSSS (SEQ ID NO: 40) |
| DARPin | DLGKKLLEAARAGQDDEVRILMANGADVNAGDPFGFTPLHLAAMFG |
| HT | HLEIVEVLLKNGADVNAHDEYGFTPLHLAAVVGHLEIVEVLLKNGAD |
| | VNACDNQGGTPLHLAAHTGHLEIVEVLLKHGADVNAQDKFGKTAFD |
| | ISIDNGNEDLAEILQSSS (SEQ ID NO: 41) |
| DARPin | DLGKKLLEAARAGQDDEVRILMANGADVNAGDPFGFTPLHLAAMFG |
| RI | HLEIVEVLLKNGADVNAHDEYGFTPLHLAAVVGHLEIVEVLLKNGAD |
| | VNACDNQGGTPLHLAARIGHLEIVEVLLKHGADVNAQDKFGKTAFDI |
| | SIDNGNEDLAEILQSSS (SEQ ID NO: 42) |
| DARPin | DLGKKLLEAARAGQDDEVRILMANGADVNAGDPFGFTPLHLAAMY |
| #45 | GHLEIVEVLLKNGADVNAHEEYGFTPLHLAAVVGHLEIVEVLLKNGA |
| | DVNACDNQGGTPLHLAAHTGHLEIVEVLLKHGADVNAQDKFGKTAF |
| | DISIDNGNEDLAEILQSSS (SEQ ID NO: 43) |
| DARPin | DLGKKLLEAARAGQDDEVRILMANGADVNAGDPFGFTPLHLAAMFG |
| HT-EI | HLEIVEVLLKNGADVNAHEEYGFTPLHLAAVVGHLEIVEVLLKNGAD |
| | VNACDNQGGTPLHLAAHTGHLEIVEVLLKHGADVNAQDKFGKTAFD |
| | ISIDIGNEDLAEILQSSS (SEQ ID NO: 44) |
| DARPin | DLGKKLLEAARAGQDDEVRILMANGADVNAGDPFGFTPLHLAAMFG |
| RM | HLEIVEVLLKNGADVNAHDEYGFTPLHLAAVVGHLEIVEVLLKNGAD |
| | VNACDNQGGTPLHLAARMGHLEIVEVLLKHGADVNAQDKFGKTAF |
| | DISIDNGNEDLAEILQSSS (SEQ ID NO: 45) |
| DARPin | DLGKKLLEAARAGQDDEVRILMANGADVNAGDPFGFTPLHLAAMFG |
| RM-E | HLEIVEVLLKNGADVNAHEEYGFTPLHLAAVVGHLEIVEVLLKNGAD |
| | VNACDNQGGTPLHLAARMGHLEIVEVLLKHGADVNAQDKFGKTAF |
| | DISIDNGNEDLAEILQSSS (SEQ ID NO: 46) |
| DARPin | DLGKKLLEAARAGQDDEVRILMANGADVNAGDPFGFTPLHLAAMFG |
| HT-ET | HLEIVEVLLKNGADVNAHEEYGFTPLHLAAVVGHLEIVEVLLKNGAD |
| | VNACDNQGGTPLHLAAHTGHLEIVEVLLKHGADVNAQDKFGKTAFD |
| | ISIDTGNEDLAEILQSSS (SEQ ID NO: 47) |
| DARPin | DLGKKLLEAARAGQDDEVRILMANGADVNAGDPFGFTPLHLAAMFG |
| HT-ERS | HLEIVEVLLKNGADVNAHEEYGFTPLHLAAVVGHLEIVEVLLKNGAD |
| | VNARDNQGGSPLHLAAHTGHLEIVEVLLKHGADVNAQDKFGKTAFD |
| | ISIDNGNEDLAEILQSSS (SEQ ID NO: 48) |
| DARPin | DLGKKLLEAARAGQDDEVRILVANGADVNAGDPFGFTPLHLAALYG |
| SHT | HLEIVEVLLKNGADVNAHEEYGFTPLHLAAVVSHMEIVEVLLNNGAD |
| | VNARDNQGGSPLHLASHTGHLEIVEVLLKQGADVNAQDKFGKTAYD |
| | ISIDIGNEDLAEILQSSS (SEQ ID NO: 49) |
| DARPin | DLGKKLLEAARAGQDDEVRILMANGADVNAGDPFGFTPLHLAAMFG |
| RI-EQ | HLEIVEVLLKNGADVNAHEEYGFTPLHLAAVVGHLEIVEVLLKNGAD |
| | VNACDNQGGTPLHLAARIGHLEIVEVLLKQGADVNAQDKFGKTAFDI |
| | SIDNGNEDLAEILQSSS (SEQ ID NO: 50) |

DARPin Dimer Proteins

| | |
|---|---|
| DARPin | DLGKKLLEAARAGQDDEVRILMANGADVNAADPAGWTPLHLAACY |
| #53 | GHLEIVGVLLKNGADVNALDWGGWTPLHLAAVIGHLEIVGVLLKHG |
| | ADVNATDLSGWTPLHLAAFRGHLEIVEVLLKHGADVNAQDKFGKTA |
| | FDISIDNGNEDLAEILQXDLGKKLLEAARAGQDDEVRILMANGADVN |
| | ARDFPGLTPLHLAAVQGHLEIVEVLLKNGADVNAVDKLGLTPLHLAA |
| | RRGHLEIVEVLLKHGADVNAVDYTGTTPLHLAAVPGHLEIVEVLLKN |
| | GADVNAQDKFGKTAFDISIDNGNEDLAEILQSSS (SEQ ID NO: 51) |
| DARPin | DLGKKLLEAARAGQDDEVRILMANGADVNARDFPGLTPLHLAAVQG |
| D2 | HLEIVDVLLKNGADVNAVDKLGLTPLHLAARRGHLEIVEVLLKHGAD |
| | VNAVDYTGTTPLHLAAVPGHLEIVEVLLKNGADVNAQDKFGKTAFDI |
| | SIDNGNEDLAEILQXDLGKKLLEAARAGQDDEVRILMANGADVNAG |
| | DPFGFTPLHLAAMFGHLEIVEVLLKNGADVNAHDEYGFTPLHLAAVV |
| | GHLEIVEVLLKNGADVNACDNQGGTPLHLAARIGHLEIVEVLLKHGA |
| | DVNAQDKFGKTAFDISIDNGNEDLAEILQSSS (SEQ ID NO: 52) |
| DARPin | DLGKKLLEAARAGQDDEVRILMANGADVNARDFPGLTPLHLAAVQG |
| D3 | HLEIVDVLLKNGADVNAVDKLGLTPLHLAARRGHLEIVEVLLKHGAD |
| | VNAVDYTGTTPLHLAAVPGHLEIVEVLLKNGADVNAQDKFGKTAFDI |
| | SIDNGNEDLAEILQXDLGKKLLEAARAGQDDEVRILMANGADVNAG |
| | DPFGFTPLHLAAMFGHLEIVEVLLKNGADVNAHDEYGFTPLHLAAVV |
| | GHLEIVEVLLKNGADVNACDNQGGTPLHLAARIGHLEIVEVLLKHGA |
| | DVNAQDKFGKTAFDISIDNGNEDLAEILQSSS (SEQ ID NO: 53) |
| DARPin | DLGKKLLEAARAGQDDEVRILMANGADVNARDFPGLTPLHLAAVQG |
| D4 | HLEIVDVLLKNGADVNAVDKLGLTPLHLAARRGHLEIVEVLLKHGAD |
| | VNAVDYTGTTPLHLAAVPGHLEIVEVLLKNGADVNAQDKFGKTAFDI |
| | SIDNGNEDLAEILQXDLGKKLLEAARAGQDDEVRILMANGADVNAG |
| | DPFGFTPLHLAAMFGHLEIVEVLLKNGADVNAHDEYGFTPLHLAAVV |
| | GHLEIVEVLLKNGADVNACDNQGGTPLHLAARIGHLEIVEVLLKHGA |
| | DVNAQDKFGKTAFDISIDNGNEDLAEILQSSS (SEQ ID NO: 54) |
| DARPin | DLGKKLLEAARAGQDDEVRILMANGADVNACDSNGRTPLHLAAVR |
| D5 | GHLEIVEVLLKNGADVNAWDVIGKTPLHLAADGGHLEIVEVLLKNG |
| | ADVNAWDLIGRTPLHLAALDGHLEIVEVLLEHGADVNAYDRPGHTPL |
| | HLAARRGHLEIVEVLLKYGADVNAQDKFGKTAFDISIDNGNEDLAEIL |
| | QXDLGKKLLEAARAGQDDEVRILMANGADVNAGDPFGFTPLHLAA |
| | MFGHLEIVEVLLKNGADVNAHDEYGFTPLHLAAVVGHLEIVEVLLKN |
| | GADVNACDNQGGTPLHLAAHTGHLEIVEVLLKHGADVNAQDKFGKT |
| | AFDISIDNGNEDLAEILQSSS (SEQ ID NO: 55) |
| DARPin | DLGKKLLEAARAGQDDEVRILMANGADVNARDFPGLTPLHLAAVQG |
| D6 | HLEIVEVLLKNGADVNAVDKLGLTPLHLAARRGHLEIVEVLLKHGAD |
| | VNAVDYTGTTPLNLAAVPGHLEIVEVLLKNGADVNAQDKFGKTAFDI |
| | SIDNGNEDLAEILQXDLGKKLLEAARAGQDDEVRILMANGADVNAG |
| | DPFGFTPLHLAAMFGHLEIVEVLLKNGADVNAHDEYGFTPLHLAAVV |
| | GHLEIVEVLLKNGADVNACDNQGGTPLHLAARIGHLEIVEVLLKHGA |
| | DVNAQDKFGKTAFDISIDNGNEDLAEILQSSS (SEQ ID NO: 56) |
| DARPin | DLGKKLLEAARAGQDDEVRILMANGADVNAGDPFGFTPLHLAAMFG |
| D7 | HLEIVEVLLKNGADVNAHDEYGFTPLHLAAVVGHLEIVEVLLKNGAD |
| | VNACDNQGGTPLHLAARIGHLEIVEVLLKHGADVNAQDKFGKTAFDI |
| | SIDNGNEDLAEILQXDLGKKLLEAARAGQDDEVRILMANGADVNAM |
| | DRHGQTPLHLAASFGHLEIVEVLLKHGADVNANDYSGGTPLHLAAIW |
| | GHLEIVGVLLKNGADVNAKDVWGRTPLHLAAIGGHLEIVEVLLKNG |
| | ADVNAQDKFGKTAFDISIDNGNEDLAEILQSSS (SEQ ID NO: 57) |
| DARPin | DLGKKLLEAARAGQDDEVRILMANGADVNAGDPFGFTPLHLAAMFG |
| D8 | HLEIVEVLLKNGADVNAHDEYGFTPLHLAAVVGHLEIVEVLLKNGAD |
| | VNACDNQGGTPLHLAARIGHLEIVEVLLKHGADVNAQDKFGKTAFDI |
| | SIDNGNEDLAEILQXDLGKKLLEAARAGQDDEVRILMANGADVNAR |
| | DESGTTPLHLAAGSGHLEIVEVLLKHGADVNAADLGGWTPLHLAAI |
| | WGHLEIVEVLLKHGADVNATDPTGWTPLHLAALGGHLEIVEVLLKN |
| | GADVNAQDKFGKTAFDISIDNGNEDLAEILQSSS (SEQ ID NO: 58) |
| DARPin | DLGKKLLEAARAGQDDEVRILMANGADVNAADPAGWTPLHLAACY |
| D9 | GHLEIVGVLLKNGADVNALDWGGWTPLHLAAVIGHLEIVGVLLKHG |
| | ADVNATDLSGWTPLHLAAFRGHLEIVEVLLKHGADVNAQDKFGKTA |
| | FDISIDNGNEDLAEILQXDLGKKLLEAARAGQDDEVRILMANGADVN |
| | ARDFPGLTPLHLAAVQGHLEIVDVLLKNGADVNAVDKLGLTPLHLA |
| | ARRGHLEIVEVLLKHGADVNAVDYTGTTPLHLAAVPGHLEIVEVLLK |
| | NGADVNAQDKFGKTAFDISIDNGNEDLAEILQSSS (SEQ ID NO: 59) |
| DARPin | DLGKKLLEAARAGQDDEVRILMANGADVNAMDRHGQTPLHLAASF |
| D10 | GHLEIVEVLLKHGADVNANDYSGGTPLHLAAIWGHLEIVGVLLKNGA |
| | DVNAKDVWGRTPLHLAAIGGHLEIVEVLLKNGADVNAQDKFGKTAF |
| | DISIDNGNEDLAEILQXDLGKKLLEAARAGQDDEVRILMANGADVNA |
| | RDFPGLTPLHLAAVQGHLEIVEVLLKNGADVNAVDKLGLTPLHLAAR |
| | RGHLEIVEVLLKHGADVNAVDYTGTTPLHLAAVPGHLEIVEVLLKNG |
| | ADVNAQDKFGKTAFDISIDNGNEDLAEILQSSS (SEQ ID NO: 60) |
| DARPin | DLGKKLLEAARAGQDDEVRILMANGADVNAGDPFGFTPLHLAAMFG |
| D11 | HLEIVEVLLKNGADVNAHDEYGFTPLHLAAVVGHLEIVEVLLKNGAD |
| | VNACDNQGGTPLHLAARIGHLEIVEVLLKHGADVNAQDKFGKTAFDI |
| | SIDNGNEDLAEILQXDLGKKLLEAARAGQDDEVRILMANGADVNAR |
| | DFPGLTPLHLAAVQGHLEIVDVLLKNGADVNAVDKLGLTPLHLAARR |
| | GHLEIVEVLLKHGADVNAVDYTGTTPLHLAAVPGHLEIVEVLLKNGA |
| | DVNAQDKFGKTAFDISIDNGNEDLAEILQSSS (SEQ ID NO: 61) |
| DARPin | DLGKKLLEAARAGQDDEVRILMANGADVNARDFPGLTPLHLAAVQG |
| D12 | HLEIVEVLLKNGADVNAVDKLGLTPLHLAARRGHLEIVEVLLKHGAD |
| | VNAVDYTGTTPLNLAAVPGHLEIVEVLLKNGADVNAQDKFGKTAFDI |
| | SIDNGNEDLAEILQXDLGKKLLEAARAGQDDEVRILMANGADVNAG |
| | DPFGFTPLHLAAMFGHLEIVEVLLKNGADVNAHDEYGFTPLHLAAVV |
| | GHLEIVEVLLKNGADVNACDNQGGTPLHLAARIGHLEIVEVLLKHGA |
| | DVNAQDKFGKTAFDISIDNGNEDLAEILQSSS (SEQ ID NO: 62) |
| DARPin | DLGKKLLEAARAGQDDEVRILMANGADVNAADPAGWTPLHLAACY |
| D13 | GHLEIVGVLLKNGADVNALDWGGWTPLHLAAVIGHLEIVGVLLKHG |
| | ADVNATDLSGWTPLHLAAFRGHLEIVEVLLKHGADVNAQDKFGKTA |
| | FDISIDNGNEDLAEILQXDLGKKLLEAARAGQDDEVRILMANGADVN |
| | ARDESGTTPLHLAAGSGHLEIVEVLLKHGADVNAADLGGWTPLHLA |
| | AIWGHLEIVEVLLKHGADVNATDPTGWTPLHLAALGGHLEIVEVLLK |
| | NGADVNAQDKFGKTAFDISIDNGNEDLAEILQSSS (SEQ ID NO: 63) |
| DARPin | DLGKKLLEAARAGQDDEVRILMANGADVNARDFPGLTPLHLAAVQG |
| D14 | HLEIVEVLLKNGADVNAVDKLGLTPLHLAARRGHLEIVEVLLKHGAD |
| | VNAVDYTGTTPLNLAAVPGHLEIVEVLLKNGADVNAQDKFGKTAFDI |
| | SIDNGNEDLAEILQXDLGKKLLEAARAGQDDEVRILMANGADVNAG |
| | DPFGFTPLHLAAMFGHLEIVEVLLKNGADVNAHDEYGFTPLHLAAVV |
| | GHLEIVEVLLKNGADVNACDNQGGTPLHLAARIGHLEIVEVLLKHGA |
| | DVNAQDKFGKTAFDISIDNGNEDLAEILQSSS (SEQ ID NO: 64) |
| DARPin | DLGKKLLEAARAGQDDEVRILMANGADVNARDFPGLTPLHLAAVQG |
| D15 | HLEIVEVLLKNGADVNAVDKLGLTPLHLAARRGHLEIVEVLLKHGAD |
| | VNAVDYTGTTPLHLAAVPGHLEIVEVLLKNGADVNAQDKFGKTAFDI |
| | SIDNGNEDLAEILQXDLGKKLLEAARAGQDDEVRILMANGADVNAG |
| | DPFGFTPLHLAAMFGHLEIVEVLLKNGADVNAHDEYGFTPLHLAAVV |
| | GHLEIVEVLLKNGADVNACDNQGGTPLHLAAHTGHLEIVEVLLKHG |
| | ADVNAQDKFGKTAFDISIDNGNEDLAEILQSSS (SEQ ID NO: 65) |
| DARPin | DLGKKLLEAARAGQDDEVRILMANGADVNAMDRHGQTPLHLAASF |
| D16 | GRLEIVEVLLKHGADVNANDYSGGTPLHLAAIWGHLEIVGVLLKNGA |
| | DVNAKDVWGRTPLHLAAIGGHLEIVEVLLKNGADVNAQDKFGKTAF |
| | DISIDNGNEDLAEILQXDLGKKLLEAARAGQDDEVRILMANGADVNA |
| | RDFPGLTPLHLAAVQGHLEIVEVLLKNGADVNAVDKLGLTPLHLAAR |
| | RGHLEIVEVLLKHGADVNAVDYTGTTPLHLAAVPGHLEIVEVLLKNG |
| | ADVNAQDKFGKTAFDISIDNGNEDLAEILQSSS (SEQ ID NO: 66) |
| DARPin | DLGKKLLEAARAGQDDEVRILMANGADVNARDFPGLTPLHLAAVQG |
| D17 | HLEIVEVLLKNGADVNAVDKLGLTPLHLAARRGHLEIVEVLLKHGAD |
| | VNAVDYTGTTPLHLAAVPGHLEIVEVLLKNGADVNAQDKFGKTAFDI |
| | SIDNGNEDLAEILQXDLGKKLLEAARAGQDDEVRILMANGADVNAG |
| | DPFGFTPLHLAAMFGHLEIVEVLLKNGADVNAHDEYGFTPLHLAAVV |
| | GHLEIVEVLLKNGADVNACDNQGGTPLHLAAHTGHLEIVEVLLKHG |
| | ADVNAQDKFGKTAFDISIDNGNEDLAEILQSSS (SEQ ID NO: 67) |
| DARPin | DLGKKLLEAARAGQDDEVRILMANGADVNARDFPGLTPLHLAAVQG |
| D18 | HLEIVEVLLKNGADVNAVDKLGLTPLHLAARRGHLEIVEVLLKHGAD |
| | VNAVDYTGTTPLNLAAVPGHLEIVEVLLKNGADVNAQDKFGKTAFDI |
| | SIDNGNEDLAEILQXDLGKKLLEAARAGQDDEVRILMANGADVNAG |
| | DPFGFTPLHLAAMFGHLEIVEVLLKNGADVNAHDEYGFTPLHLAAVV |
| | GHLEIVEVLLKNGADVNACDNQGGTPLHLAARIGHLEIVEVLLKHGA |
| | DVNAQDKFGKTAFDISIDNGNEDLAEILQSSS (SEQ ID NO: 68) |
| DARPin | DLGKKLLEAARAGQDDEVRILMANGADVNARDFPGLTPLHLAAVQG |
| D19 | HLEIVDVLLKNGADVNAVDKLGLTPLHLAARRGHLEIVEVLLKHGAD |
| | VNAVDYTGTTPLHLAAVPGHLEIVEVLLKNGADVNAQDKFGKTAFDI |
| | SIDNGNEDLAEILQXDLGKKLLEAARAGQDDEVRILMANGADVNAG |
| | DPFGFTPLHLAAMFGHLEIVEVLLKNGADVNAHDEYGFTPLHLAAVV |
| | GHLEIVEVLLKNGADVNACDNQGGTPLHLAARIGHLEIVEVLLKHGA |
| | DVNAQDKFGKTAFDISIDNGNEDLAEILQSSS (SEQ ID NO: 69) |
| DARPin | DLGKKLLEAARAGQDDEVRILMANGADVNARDFPGLTPLHLAAVQG |
| D20 | HLEIVEVLLKNGADVNAVDKLGLTPLHLAARRGHLEIVEVLLKHGAD |
| | VNAVDYTGTTPLHLAAVPGHLEIVEVLLKNGADVNAQDKFGKTAFDI |
| | SIDNGNEDLAEILQXDLGKKLLEAARAGQDDEVRILMANGADVNAG |
| | DPFGFTPLHLAAMFGHLEIVEVLLKNAADVNAHDEYGFTPLHLAAVV |
| | GHLEIVEVLLKNGADVNACDNQGGTPLHLAARIGHLEIVEVLLKHGA |
| | DVNAQDKFGKTAFDISIDNGNEDLAEILQSSS (SEQ ID NO: 70) |
| DARPin | DLGKKLLEAARAGQDDEVRILMANGADVNACDSNGRTPLHLAAVR |
| SD5 | GHLEIVEVLLKNGADVNAWDVIGKTPLHLAADGGHLEIVEVLLKNG |
| | ADVNAWDLIGRTPLHLAALDGHLEIVEVLLEHGADVNAYDRPGHTPL |
| | HLAARRGHLEIVEVLLKYGADVNAQDKFGKTAFDISIDNGNEDLAEIL |
| | QXDLGKKLLEAARAGQDDEVRILVANGADVNAGDPFGFTPLHLAAL |
| | YGHLEIVEVLLKNGADVNAHEEYGFTPLHLAAVVSHMEIVEVLLNNG |
| | ADVNARDNQGGSPLHLASHTGHLEIVEVLLKQGADVNAQDKFGKTA |
| | YDISIDIGNEDLAEILQSSS (SEQ ID NO: 71) |

In an illustrative aspect, a monomer protein is provided. In an embodiment, the monomer protein comprises an amino acid sequence of SEQ ID NO: 1. In an embodiment, the monomer protein comprises an amino acid sequence of SEQ ID NO: 2. In an embodiment, the monomer protein comprises an amino acid sequence of SEQ ID NO: 3. In an embodiment, the monomer protein comprises an amino acid sequence of SEQ ID NO: 4. In an embodiment, the monomer protein comprises an amino acid sequence of SEQ ID NO: 5. In an embodiment, the monomer protein comprises an amino acid sequence of SEQ ID NO: 6. In an embodiment, the monomer protein comprises an amino acid sequence of SEQ ID NO: 7. In an embodiment, the monomer protein comprises an amino acid sequence of SEQ ID NO: 8. In an embodiment, the monomer protein comprises an amino acid sequence of SEQ ID NO: 9. In an embodiment, the monomer protein comprises an amino acid sequence of SEQ ID NO: 10. In an embodiment, the monomer protein comprises an amino acid sequence of SEQ ID NO: 11. In an embodiment, the monomer protein comprises an amino acid sequence of SEQ ID NO: 12. In an embodiment, the monomer protein comprises an amino acid sequence of SEQ ID NO: 13. In an embodiment, the monomer protein comprises an amino acid sequence of SEQ ID NO: 14. In an embodiment, the monomer protein comprises an amino acid sequence of SEQ ID NO: 15. In an embodiment, the monomer protein comprises an amino acid sequence of SEQ ID NO: 16. In an embodiment, the monomer protein comprises an amino acid sequence of SEQ ID NO: 17. In an embodiment, the monomer protein comprises an amino acid sequence of SEQ ID NO: 18. In an embodiment, the monomer protein comprises an amino acid sequence of SEQ ID NO: 19. In an embodiment, the monomer protein comprises an amino acid sequence of SEQ ID NO: 20. In an embodiment, the monomer protein comprises an amino acid sequence of SEQ ID NO: 21. In an embodiment, the monomer protein comprises an amino acid sequence of SEQ ID NO: 22. In an embodiment, the monomer protein comprises an amino acid sequence of SEQ ID NO: 23. In an embodiment, the monomer protein comprises an amino acid sequence of SEQ ID NO: 24. In an embodiment, the monomer protein comprises an amino acid sequence of SEQ ID NO: 25. In an embodiment, the monomer protein comprises an amino acid sequence of SEQ ID NO: 26. In an embodiment, the monomer protein comprises an amino acid sequence of SEQ ID NO: 27. In an embodiment, the monomer protein comprises an amino acid sequence of SEQ ID NO: 28. In an embodiment, the monomer protein comprises an amino acid sequence of SEQ ID NO: 29. In an embodiment, the monomer protein comprises an amino acid sequence of SEQ ID NO: 30. In an embodiment, the monomer protein comprises an amino acid sequence of SEQ ID NO: 31. In an embodiment, the monomer protein comprises an amino acid sequence of SEQ ID NO: 32. In an embodiment, the monomer protein comprises an amino acid sequence of SEQ ID NO: 33. In an embodiment, the monomer protein comprises an amino acid sequence of SEQ ID NO: 34. In an embodiment, the monomer protein comprises an amino acid sequence of SEQ ID NO: 35. In an embodiment, the monomer protein comprises an amino acid sequence of SEQ ID NO: 36. In an embodiment, the monomer protein comprises an amino acid sequence of SEQ ID NO: 37. In an embodiment, the monomer protein comprises an amino acid sequence of SEQ ID NO: 38. In an embodiment, the monomer protein comprises an amino acid sequence of SEQ ID NO: 39. In an embodiment, the monomer protein comprises an amino acid sequence of SEQ ID NO: 40. In an embodiment, the monomer protein comprises an amino acid sequence of SEQ ID NO: 41. In an embodiment, the monomer protein comprises an amino acid sequence of SEQ ID NO: 42. In an embodiment, the monomer protein comprises an amino acid sequence of SEQ ID NO: 43. In an embodiment, the monomer protein comprises an amino acid sequence of SEQ ID NO: 44. In an embodiment, the monomer protein comprises an amino acid sequence of SEQ ID NO: 45. In an embodiment, the monomer protein comprises an amino acid sequence of SEQ ID NO: 46. In an embodiment, the monomer protein comprises an amino acid sequence of SEQ ID NO: 47. In an embodiment, the monomer protein comprises an amino acid sequence of SEQ ID NO: 48. In an embodiment, the monomer protein comprises an amino acid sequence of SEQ ID NO: 49. In an embodiment, the monomer protein comprises an amino acid sequence of SEQ ID NO: 50.

In an embodiment, the monomer protein further comprises a tagging molecule at the N-terminus of the amino acid sequence. As used herein, a “tagging molecule” refers to one or more amino acids that are utilized for identification of an amino acid, for instance His-tag and Myc-tag. Such tagging molecules are well known in the art. In an embodiment, the tagging molecule comprises MGSSHHHHHHSSGLVPRGSHMEQKLISEEDLGS (SEQ ID NO: 72). In an embodiment, the tagging molecule consists of MGSSHHHHHHSSGLVPRGSHMEQKLISEEDLGS (SEQ ID NO: 72). In an embodiment, the tagging molecule consists essentially of

(SEQ ID NO: 72)
MGSSHHHHHHSSGLVPRGSHMEQKLISEEDLGS.

In alternative embodiments, the monomer protein consists essentially of the amino acid sequence as described herein (e.g., one of SEQ ID NO: 1 to SEQ ID NO: 50). In other alternative embodiments, the monomer protein consists of the amino acid sequence as described herein (e.g., one of SEQ ID NO: 1 to SEQ ID NO: 50).

In an illustrative aspect, a dimer protein is provided. In an embodiment, the dimer protein comprises an amino acid sequence of SEQ ID NO: 51, wherein X represents a linker. In an embodiment, the dimer protein comprises an amino acid sequence of SEQ ID NO: 52, wherein X represents a linker. In an embodiment, the dimer protein comprises an amino acid sequence of SEQ ID NO: 53, wherein X represents a linker. In an embodiment, the dimer protein comprises an amino acid sequence of SEQ ID NO: 54, wherein X represents a linker. In an embodiment, the dimer protein comprises an amino acid sequence of SEQ ID NO: 55, wherein X represents a linker. In an embodiment, the dimer protein comprises an amino acid sequence of SEQ ID NO: 56, wherein X represents a linker. In an embodiment, the dimer protein comprises an amino acid sequence of SEQ ID NO: 57, wherein X represents a linker. In an embodiment, the dimer protein comprises an amino acid sequence of SEQ ID NO: 58, wherein X represents a linker. In an embodiment, the dimer protein comprises an amino acid sequence of SEQ ID NO: 59, wherein X represents a linker. In an embodiment, the dimer protein comprises an amino acid sequence of SEQ ID NO: 60, wherein X represents a linker. In an embodiment, the dimer protein comprises an amino acid sequence of SEQ ID NO: 61, wherein X represents a linker. In an embodiment, the dimer protein comprises an amino acid sequence of SEQ ID NO: 62, wherein X represents a linker. In an embodiment, the dimer protein comprises an amino acid sequence of SEQ ID NO: 63, wherein X represents a linker. In an embodiment, the dimer protein comprises an amino acid sequence of SEQ ID NO: 64, wherein X represents a linker. In an embodiment, the dimer protein comprises an amino acid sequence of SEQ ID NO: 65, wherein X represents a linker. In an embodiment, the dimer protein comprises an amino acid sequence of SEQ ID NO: 66, wherein X represents a linker. In an embodiment, the dimer protein comprises an amino acid sequence of SEQ ID NO: 67, wherein X represents a linker. In an embodiment, the dimer protein comprises an amino acid sequence of SEQ ID NO: 68, wherein X represents a linker. In an embodiment, the dimer protein comprises an amino acid sequence of SEQ ID NO: 69, wherein X represents a linker. In an embodiment, the dimer protein comprises an amino acid sequence of SEQ ID NO: 70, wherein X represents a linker. In an embodiment, the dimer protein comprises an amino acid sequence of SEQ ID NO: 71, wherein X represents a linker.

As used herein, a “linker” refers to one or more amino acids that are utilized to link one monomer to another monomer. Such linkers are well known in the art. In an embodiment, the linker is selected from the group consisting of (GS)n, (GGGGS)n, (PS)n, and (PT)n, wherein n represents any number between 2-100. In an embodiment, the linker is (G₄S)x4. In an embodiment, the linker comprises SSSGGGGSEFGGGGSGGGGSGGGGSAS (SEQ ID NO: 73). In an embodiment, the linker consists essentially of SSSGGGGSEFGGGGSGGGGSGGGGSAS (SEQ ID NO: 73). In an embodiment, the linker consists of SSSGGGGSEFGGGGSGGGGSGGGGSAS (SEQ ID NO: 73).

In an illustrative aspect, a dimer protein is provided in which the dimer protein comprises a first monomer protein and a second monomer protein. Any of the described monomer proteins can be utilized as the first monomer protein, the second monomer protein, or both. In an embodiment, the first monomer protein and the second monomer protein are connected via a linker. The linker for these embodiments can be any of the linkers described herein.

In an illustrative aspect, a trimer protein is provided in which the trimer protein comprises a first monomer protein, a second monomer protein, and a third monomer protein. Any of the described monomer proteins can be utilized as the first monomer protein, the second monomer protein, the third monomer protein, or any combination thereof. In an embodiment, the first monomer protein and the second monomer protein are connected via a linker. In an embodiment, the second monomer protein and the third monomer protein are connected via a second linker. The linker for these embodiments can be any of the linkers described herein.

In an illustrative aspect, a pharmaceutical composition comprising a monomer protein is provided. Any of the described monomer proteins can be utilized as the monomer protein in this aspect. In another illustrative aspect, a pharmaceutical composition comprising a dimer protein is provided. Any of the described dimer proteins can be utilized as the dimer protein in this aspect. In yet another illustrative aspect, a pharmaceutical composition comprising a trimer protein is provided. Any of the described trimer proteins can be utilized as the trimer protein in this aspect.

In an embodiment, the pharmaceutical composition is a parenteral formulation. In an embodiment, the parenteral formulation is selected from the group consisting of intravenous, intraarterial, intraperitoneal, intrathecal, intradermal, epidural, intracerebroventricular, intraurethral, intrasternal, intracranial, intratumoral, intramuscular and subcutaneous.

In an embodiment, the pharmaceutical composition comprises one or more pharmaceutically acceptable carriers. In an embodiment, the pharmaceutical composition further comprises a second therapeutic agent. In an embodiment, the pharmaceutical composition is formulated as a single dose. In an embodiment, the pharmaceutical composition is formulated as a single unit dose.

In an illustrative aspect, a method of treating a Shiga toxin-producing Escherichia coli (STEC) infection in a patient is provided. The method comprises the step of administering a therapeutically effective amount of a pharmaceutical composition to the patient. Any of the described pharmaceutical compositions can be utilized as the pharmaceutical composition in this aspect.

In an embodiment, the pharmaceutical composition reduces one or more symptoms of the STEC infection in the patient. In an embodiment, the one or more symptoms comprise a gastrointestinal symptom. In an embodiment, the one or more symptoms comprise diarrhea. In an embodiment, the one or more symptoms comprise bloody diarrhea. In an embodiment, the one or more symptoms comprise stomach cramps. In an embodiment, the one or more symptoms comprise vomiting. In an embodiment, the one or more symptoms comprise hemolytic uremic syndrome (HUS).

In an embodiment, the method further comprises administration of an antibiotic to the patient. In an embodiment, the method further comprises administration of intravenous fluids to the patient.

In an embodiment, the administration is a parenteral administration. In an embodiment, the parenteral administration is selected from the group consisting of intravenous, intraarterial, intraperitoneal, intrathecal, intradermal, epidural, intracerebroventricular, intraurethral, intrasternal, intracranial, intratumoral, intramuscular and subcutaneous. In an embodiment, the parenteral administration is intravenous. In an embodiment, the parenteral administration is intraarterial. In an embodiment, the parenteral administration is intraperitoneal. In an embodiment, the parenteral administration is intrathecal. In an embodiment, the parenteral administration is intradermal. In an embodiment, the parenteral administration is epidural. In an embodiment, the parenteral administration is intracerebroventricular. In an embodiment, the parenteral administration is intraurethral. In an embodiment, the parenteral administration is intrasternal. In an embodiment, the parenteral administration is intracranial. In an embodiment, the parenteral administration is intramuscular. In an embodiment, the parenteral administration is subcutaneous.

In an illustrative aspect, a method of treating a hemolytic uremic syndrome (HUS) in a patient is provided. The method comprises the step of administering a therapeutically effective amount of a pharmaceutical composition to the patient. Any of the described pharmaceutical compositions can be utilized as the pharmaceutical composition in this aspect.

In an embodiment, the patient is a child patient. In an embodiment, the patient is an elderly patient.

In an embodiment, the method further comprises administration of an antibiotic to the patient. In an embodiment, the method further comprises administration of intravenous fluids to the patient. In an embodiment, the method further comprises administration of blood transfusion to the patient. In an embodiment, the method further comprises administration of plasma exchange to the patient. In an embodiment, the method further comprises administration of kidney dialysis to the patient.

In an embodiment, the administration is a parenteral administration. In an embodiment, the parenteral administration is selected from the group consisting of intravenous, intraarterial, intraperitoneal, intrathecal, intradermal, epidural, intracerebroventricular, intraurethral, intrasternal, intracranial, intratumoral, intramuscular and subcutaneous. In an embodiment, the parenteral administration is intravenous. In an embodiment, the parenteral administration is intraarterial. In an embodiment, the parenteral administration is intraperitoneal. In an embodiment, the parenteral administration is intrathecal. In an embodiment, the parenteral administration is intradermal. In an embodiment, the parenteral administration is epidural. In an embodiment, the parenteral administration is intracerebroventricular. In an embodiment, the parenteral administration is intraurethral. In an embodiment, the parenteral administration is intrasternal. In an embodiment, the parenteral administration is intracranial. In an embodiment, the parenteral administration is intramuscular. In an embodiment, the parenteral administration is subcutaneous.

The present disclosure expressly incorporates the journal article of Zeng et al., “A Multi-Specific DARPin Potently Neutralizes Shiga Toxin 2 via Simultaneous Modulation of Both Toxin Subunits,” Bioengineering, 2022, 9, starting at 511, in its entirety.

The following numbered embodiments are contemplated and are non-limiting:
1. A monomer protein comprising an amino acid sequence of SEQ ID NO: 1.
2. A monomer protein comprising an amino acid sequence of SEQ ID NO: 2.
3. A monomer protein comprising an amino acid sequence of SEQ ID NO: 3.
4. A monomer protein comprising an amino acid sequence of SEQ ID NO: 4.
5. A monomer protein comprising an amino acid sequence of SEQ ID NO: 5.
6. A monomer protein comprising an amino acid sequence of SEQ ID NO: 6.
7. A monomer protein comprising an amino acid sequence of SEQ ID NO: 7.
8. A monomer protein comprising an amino acid sequence of SEQ ID NO: 8.
9. A monomer protein comprising an amino acid sequence of SEQ ID NO: 9.
10. A monomer protein comprising an amino acid sequence of SEQ ID NO: 10.
11. A monomer protein comprising an amino acid sequence of SEQ ID NO: 11.
12. A monomer protein comprising an amino acid sequence of SEQ ID NO: 12.
13. A monomer protein comprising an amino acid sequence of SEQ ID NO: 13.
14. A monomer protein comprising an amino acid sequence of SEQ ID NO: 14.
15. A monomer protein comprising an amino acid sequence of SEQ ID NO: 15.
16. A monomer protein comprising an amino acid sequence of SEQ ID NO: 16.
17. A monomer protein comprising an amino acid sequence of SEQ ID NO: 17.
18. A monomer protein comprising an amino acid sequence of SEQ ID NO: 18.
19. A monomer protein comprising an amino acid sequence of SEQ ID NO: 19.
20. A monomer protein comprising an amino acid sequence of SEQ ID NO: 20.
21. A monomer protein comprising an amino acid sequence of SEQ ID NO: 21.
22. A monomer protein comprising an amino acid sequence of SEQ ID NO: 22.
23. A monomer protein comprising an amino acid sequence of SEQ ID NO: 23.
24. A monomer protein comprising an amino acid sequence of SEQ ID NO: 24.
25. A monomer protein comprising an amino acid sequence of SEQ ID NO: 25.
26. A monomer protein comprising an amino acid sequence of SEQ ID NO: 26.
27. A monomer protein comprising an amino acid sequence of SEQ ID NO: 27.
28. A monomer protein comprising an amino acid sequence of SEQ ID NO: 28.
29. A monomer protein comprising an amino acid sequence of SEQ ID NO: 29.
30. A monomer protein comprising an amino acid sequence of SEQ ID NO: 30.
31. A monomer protein comprising an amino acid sequence of SEQ ID NO: 31.
32. A monomer protein comprising an amino acid sequence of SEQ ID NO: 32.
33. A monomer protein comprising an amino acid sequence of SEQ ID NO: 33.
34. A monomer protein comprising an amino acid sequence of SEQ ID NO: 34.
35. A monomer protein comprising an amino acid sequence of SEQ ID NO: 35.
36. A monomer protein comprising an amino acid sequence of SEQ ID NO: 36.
37. A monomer protein comprising an amino acid sequence of SEQ ID NO: 37.
38. A monomer protein comprising an amino acid sequence of SEQ ID NO: 38.
39. A monomer protein comprising an amino acid sequence of SEQ ID NO: 39.
40. A monomer protein comprising an amino acid sequence of SEQ ID NO: 40.
41. A monomer protein comprising an amino acid sequence of SEQ ID NO: 41.
42. A monomer protein comprising an amino acid sequence of SEQ ID NO: 42.
43. A monomer protein comprising an amino acid sequence of SEQ ID NO: 43.
44. A monomer protein comprising an amino acid sequence of SEQ ID NO: 44.
45. A monomer protein comprising an amino acid sequence of SEQ ID NO: 45.
46. A monomer protein comprising an amino acid sequence of SEQ ID NO: 46.
47. A monomer protein comprising an amino acid sequence of SEQ ID NO: 47.
48. A monomer protein comprising an amino acid sequence of SEQ ID NO: 48.
49. A monomer protein comprising an amino acid sequence of SEQ ID NO: 49.
50. A monomer protein comprising an amino acid sequence of SEQ ID NO: 50.
51. The monomer protein of any one of clauses 1 to 50, any other suitable clause, or any combination of suitable clauses, wherein the monomer protein further comprises a tagging molecule at the N-terminus of the amino acid sequence.
52. The monomer protein of clause 51, any other suitable clause, or any combination of suitable clauses, wherein the tagging molecule comprises

(SEQ ID NO: 72)
MGSSHHHHHHSSGLVPRGSHMEQKLISEEDLGS.

53. The monomer protein of clause 51, any other suitable clause, or any combination of suitable clauses, wherein the tagging molecule consists of

(SEQ ID NO: 72)
MGSSHHHHHHSSGLVPRGSHMEQKLISEEDLGS.

54. The monomer protein of clause 51, any other suitable clause, or any combination of suitable clauses, wherein the tagging molecule consists essentially of

(SEQ ID NO: 72)
MGSSHHHHHHSSGLVPRGSHMEQKLISEEDLGS.

55. A monomer protein consisting essentially of an amino acid sequence of SEQ ID NO: 1.
56. A monomer protein consisting essentially of an amino acid sequence of SEQ ID NO: 2.
57. A monomer protein consisting essentially of an amino acid sequence of SEQ ID NO: 3.
58. A monomer protein consisting essentially of an amino acid sequence of SEQ ID NO: 4.
59. A monomer protein consisting essentially of an amino acid sequence of SEQ ID NO: 5.
60. A monomer protein consisting essentially of an amino acid sequence of SEQ ID NO: 6.
61. A monomer protein consisting essentially of an amino acid sequence of SEQ ID NO: 7.
62. A monomer protein consisting essentially of an amino acid sequence of SEQ ID NO: 8.
63. A monomer protein consisting essentially of an amino acid sequence of SEQ ID NO: 9.
64. A monomer protein consisting essentially of an amino acid sequence of SEQ ID NO: 10.
65. A monomer protein consisting essentially of an amino acid sequence of SEQ ID NO: 11.
66. A monomer protein consisting essentially of an amino acid sequence of SEQ ID NO: 12.
67. A monomer protein consisting essentially of an amino acid sequence of SEQ ID NO: 13.
68. A monomer protein consisting essentially of an amino acid sequence of SEQ ID NO: 14.
69. A monomer protein consisting essentially of an amino acid sequence of SEQ ID NO: 15.
70. A monomer protein consisting essentially of an amino acid sequence of SEQ ID NO: 16.
71. A monomer protein consisting essentially of an amino acid sequence of SEQ ID NO: 17.
72. A monomer protein consisting essentially of an amino acid sequence of SEQ ID NO: 18.
73. A monomer protein consisting essentially of an amino acid sequence of SEQ ID NO: 19.
74. A monomer protein consisting essentially of an amino acid sequence of SEQ ID NO: 20.
75. A monomer protein consisting essentially of an amino acid sequence of SEQ ID NO: 21.
76. A monomer protein consisting essentially of an amino acid sequence of SEQ ID NO: 22.
77. A monomer protein consisting essentially of an amino acid sequence of SEQ ID NO: 23.
78. A monomer protein consisting essentially of an amino acid sequence of SEQ ID NO: 24.
79. A monomer protein consisting essentially of an amino acid sequence of SEQ ID NO: 25.
80. A monomer protein consisting essentially of an amino acid sequence of SEQ ID NO: 26.
81. A monomer protein consisting essentially of an amino acid sequence of SEQ ID NO: 27.
82. A monomer protein consisting essentially of an amino acid sequence of SEQ ID NO: 28.
83. A monomer protein consisting essentially of an amino acid sequence of SEQ ID NO: 29.
84. A monomer protein consisting essentially of an amino acid sequence of SEQ ID NO: 30.
85. A monomer protein consisting essentially of an amino acid sequence of SEQ ID NO: 31.
86. A monomer protein consisting essentially of an amino acid sequence of SEQ ID NO: 32.
87. A monomer protein consisting essentially of an amino acid sequence of SEQ ID NO: 33.
88. A monomer protein consisting essentially of an amino acid sequence of SEQ ID NO: 34.
89. A monomer protein consisting essentially of an amino acid sequence of SEQ ID NO: 35.
90. A monomer protein consisting essentially of an amino acid sequence of SEQ ID NO: 36.
91. A monomer protein consisting essentially of an amino acid sequence of SEQ ID NO: 37.
92. A monomer protein consisting essentially of an amino acid sequence of SEQ ID NO: 38.
93. A monomer protein consisting essentially of an amino acid sequence of SEQ ID NO: 39.
94. A monomer protein consisting essentially of an amino acid sequence of SEQ ID NO: 40.
95. A monomer protein consisting essentially of an amino acid sequence of SEQ ID NO: 41.
96. A monomer protein consisting essentially of an amino acid sequence of SEQ ID NO: 42.
97. A monomer protein consisting essentially of an amino acid sequence of SEQ ID NO: 43.
98. A monomer protein consisting essentially of an amino acid sequence of SEQ ID NO: 44.
99. A monomer protein consisting essentially of an amino acid sequence of SEQ ID NO: 45.
100. A monomer protein consisting essentially of an amino acid sequence of SEQ ID NO: 46.
101. A monomer protein consisting essentially of an amino acid sequence of SEQ ID NO: 47.
102. A monomer protein consisting essentially of an amino acid sequence of SEQ ID NO: 48.
103. A monomer protein consisting essentially of an amino acid sequence of SEQ ID NO: 49.
102. A monomer protein consisting essentially of an amino acid sequence of SEQ ID NO: 50.
105. A monomer protein consisting of an amino acid sequence of SEQ ID NO: 1.
106. A monomer protein consisting of an amino acid sequence of SEQ ID NO: 2.
107. A monomer protein consisting of an amino acid sequence of SEQ ID NO: 3.
108. A monomer protein consisting of an amino acid sequence of SEQ ID NO: 4.
109. A monomer protein consisting of an amino acid sequence of SEQ ID NO: 5.
110. A monomer protein consisting of an amino acid sequence of SEQ ID NO: 6.
111. A monomer protein consisting of an amino acid sequence of SEQ ID NO: 7.
112. A monomer protein consisting of an amino acid sequence of SEQ ID NO: 8.
113. A monomer protein consisting of an amino acid sequence of SEQ ID NO: 9.
114. A monomer protein consisting of an amino acid sequence of SEQ ID NO: 10.
115. A monomer protein consisting of an amino acid sequence of SEQ ID NO: 11.
116. A monomer protein consisting of an amino acid sequence of SEQ ID NO: 12.
117. A monomer protein consisting of an amino acid sequence of SEQ ID NO: 13.
118. A monomer protein consisting of an amino acid sequence of SEQ ID NO: 14.
119. A monomer protein consisting of an amino acid sequence of SEQ ID NO: 15.
120. A monomer protein consisting of an amino acid sequence of SEQ ID NO: 16.
121. A monomer protein consisting of an amino acid sequence of SEQ ID NO: 17.
122. A monomer protein consisting of an amino acid sequence of SEQ ID NO: 18.
123. A monomer protein consisting of an amino acid sequence of SEQ ID NO: 19.
124. A monomer protein consisting of an amino acid sequence of SEQ ID NO: 20.
125. A monomer protein consisting of an amino acid sequence of SEQ ID NO: 21.
126. A monomer protein consisting of an amino acid sequence of SEQ ID NO: 22.
127. A monomer protein consisting of an amino acid sequence of SEQ ID NO: 23.
128. A monomer protein consisting of an amino acid sequence of SEQ ID NO: 24.
129. A monomer protein consisting of an amino acid sequence of SEQ ID NO: 25.
130. A monomer protein consisting of an amino acid sequence of SEQ ID NO: 26.
131. A monomer protein consisting of an amino acid sequence of SEQ ID NO: 27.
132. A monomer protein consisting of an amino acid sequence of SEQ ID NO: 28.
133. A monomer protein consisting of an amino acid sequence of SEQ ID NO: 29.
134. A monomer protein consisting of an amino acid sequence of SEQ ID NO: 30.
135. A monomer protein consisting of an amino acid sequence of SEQ ID NO: 31.
136. A monomer protein consisting of an amino acid sequence of SEQ ID NO: 32.
137. A monomer protein consisting of an amino acid sequence of SEQ ID NO: 33.
138. A monomer protein consisting of an amino acid sequence of SEQ ID NO: 34.
139. A monomer protein consisting of an amino acid sequence of SEQ ID NO: 35.
140. A monomer protein consisting of an amino acid sequence of SEQ ID NO: 36.
141. A monomer protein consisting of an amino acid sequence of SEQ ID NO: 37.
142. A monomer protein consisting of an amino acid sequence of SEQ ID NO: 38.
143. A monomer protein consisting of an amino acid sequence of SEQ ID NO: 39.
144. A monomer protein consisting of an amino acid sequence of SEQ ID NO: 40.
145. A monomer protein consisting of an amino acid sequence of SEQ ID NO: 41.
146. A monomer protein consisting of an amino acid sequence of SEQ ID NO: 42.
147. A monomer protein consisting of an amino acid sequence of SEQ ID NO: 43.
148. A monomer protein consisting of an amino acid sequence of SEQ ID NO: 44.
149. A monomer protein consisting of an amino acid sequence of SEQ ID NO: 45.
150. A monomer protein consisting of an amino acid sequence of SEQ ID NO: 46.
151. A monomer protein consisting of an amino acid sequence of SEQ ID NO: 47.
152. A monomer protein consisting of an amino acid sequence of SEQ ID NO: 48.
153. A monomer protein consisting of an amino acid sequence of SEQ ID NO: 49.
154. A monomer protein consisting of an amino acid sequence of SEQ ID NO: 50.
155. A dimer protein comprising an amino acid sequence of SEQ ID NO: 51, any other suitable clause, or any combination of suitable clauses, wherein X represents a linker.
156. A dimer protein comprising an amino acid sequence of SEQ ID NO: 52, any other suitable clause, or any combination of suitable clauses, wherein X represents a linker.
157. A dimer protein comprising an amino acid sequence of SEQ ID NO: 53, any other suitable clause, or any combination of suitable clauses, wherein X represents a linker.
158. A dimer protein comprising an amino acid sequence of SEQ ID NO: 54, any other suitable clause, or any combination of suitable clauses, wherein X represents a linker.
159. A dimer protein comprising an amino acid sequence of SEQ ID NO: 55, any other suitable clause, or any combination of suitable clauses, wherein X represents a linker.
160. A dimer protein comprising an amino acid sequence of SEQ ID NO: 56, any other suitable clause, or any combination of suitable clauses, wherein X represents a linker.
161. A dimer protein comprising an amino acid sequence of SEQ ID NO: 57, any other suitable clause, or any combination of suitable clauses, wherein X represents a linker.
162. A dimer protein comprising an amino acid sequence of SEQ ID NO: 58, any other suitable clause, or any combination of suitable clauses, wherein X represents a linker.
163. A dimer protein comprising an amino acid sequence of SEQ ID NO: 59, any other suitable clause, or any combination of suitable clauses, wherein X represents a linker.
164. A dimer protein comprising an amino acid sequence of SEQ ID NO: 60, any other suitable clause, or any combination of suitable clauses, wherein X represents a linker.
165. A dimer protein comprising an amino acid sequence of SEQ ID NO: 61, any other suitable clause, or any combination of suitable clauses, wherein X represents a linker.
166. A dimer protein comprising an amino acid sequence of SEQ ID NO: 62, any other suitable clause, or any combination of suitable clauses, wherein X represents a linker.
167. A dimer protein comprising an amino acid sequence of SEQ ID NO: 63, any other suitable clause, or any combination of suitable clauses, wherein X represents a linker.
168. A dimer protein comprising an amino acid sequence of SEQ ID NO: 64, any other suitable clause, or any combination of suitable clauses, wherein X represents a linker.
169. A dimer protein comprising an amino acid sequence of SEQ ID NO: 65, any other suitable clause, or any combination of suitable clauses, wherein X represents a linker.
170. A dimer protein comprising an amino acid sequence of SEQ ID NO: 66, any other suitable clause, or any combination of suitable clauses, wherein X represents a linker.
171. A dimer protein comprising an amino acid sequence of SEQ ID NO: 67, any other suitable clause, or any combination of suitable clauses, wherein X represents a linker.
172. A dimer protein comprising an amino acid sequence of SEQ ID NO: 68, any other suitable clause, or any combination of suitable clauses, wherein X represents a linker.
173. A dimer protein comprising an amino acid sequence of SEQ ID NO: 69, any other suitable clause, or any combination of suitable clauses, wherein X represents a linker.
174. A dimer protein comprising an amino acid sequence of SEQ ID NO: 70, any other suitable clause, or any combination of suitable clauses, wherein X represents a linker.
175. A dimer protein comprising an amino acid sequence of SEQ ID NO: 71, any other suitable clause, or any combination of suitable clauses, wherein X represents a linker.
176. The dimer protein of any one of clauses 155 to 175, any other suitable clause, or any combination of suitable clauses, wherein the dimer protein further comprises a tagging molecule at the N-terminus of the amino acid sequence.
177. The dimer protein of clause 176, any other suitable clause, or any combination of suitable clauses, wherein the molecule tagging comprises

(SEQ ID NO: 72)
MGSSHHHHHHSSGLVPRGSHMEQKLISEEDLGS.

178. The dimer protein of clause 176, any other suitable clause, or any combination of suitable clauses, wherein the tagging molecule consists of

(SEQ ID NO: 72)
MGSSHHHHHHSSGLVPRGSHMEQKLISEEDLGS.

179. The dimer protein of clause 176, any other suitable clause, or any combination of suitable clauses, wherein the tagging molecule consists essentially of

(SEQ ID NO: 72)
MGSSHHHHHHSSGLVPRGSHMEQKLISEEDLGS.

180. A dimer protein consisting essentially of an amino acid sequence of SEQ ID NO: 51, any other suitable clause, or any combination of suitable clauses, wherein X represents a linker.
181. A dimer protein consisting essentially of an amino acid sequence of SEQ ID NO: 52, any other suitable clause, or any combination of suitable clauses, wherein X represents a linker.
182. A dimer protein consisting essentially of an amino acid sequence of SEQ ID NO: 53, any other suitable clause, or any combination of suitable clauses, wherein X represents a linker.
183. A dimer protein consisting essentially of an amino acid sequence of SEQ ID NO: 54, any other suitable clause, or any combination of suitable clauses, wherein X represents a linker.
184. A dimer protein consisting essentially of an amino acid sequence of SEQ ID NO: 55, any other suitable clause, or any combination of suitable clauses, wherein X represents a linker.
185. A dimer protein consisting essentially of an amino acid sequence of SEQ ID NO: 56, any other suitable clause, or any combination of suitable clauses, wherein X represents a linker.
186. A dimer protein consisting essentially of an amino acid sequence of SEQ ID NO: 57, any other suitable clause, or any combination of suitable clauses, wherein X represents a linker.
187. A dimer protein consisting essentially of an amino acid sequence of SEQ ID NO: 58, any other suitable clause, or any combination of suitable clauses, wherein X represents a linker.
188. A dimer protein consisting essentially of an amino acid sequence of SEQ ID NO: 59, any other suitable clause, or any combination of suitable clauses, wherein X represents a linker.
189. A dimer protein consisting essentially of an amino acid sequence of SEQ ID NO: 60, any other suitable clause, or any combination of suitable clauses, wherein X represents a linker.
190. A dimer protein consisting essentially of an amino acid sequence of SEQ ID NO: 61, any other suitable clause, or any combination of suitable clauses, wherein X represents a linker.
191. A dimer protein consisting essentially of an amino acid sequence of SEQ ID NO: 62, any other suitable clause, or any combination of suitable clauses, wherein X represents a linker.
192. A dimer protein consisting essentially of an amino acid sequence of SEQ ID NO: 63, any other suitable clause, or any combination of suitable clauses, wherein X represents a linker.
193. A dimer protein consisting essentially of an amino acid sequence of SEQ ID NO: 64, any other suitable clause, or any combination of suitable clauses, wherein X represents a linker.
194. A dimer protein consisting essentially of an amino acid sequence of SEQ ID NO: 65, any other suitable clause, or any combination of suitable clauses, wherein X represents a linker.
195. A dimer protein consisting essentially of an amino acid sequence of SEQ ID NO: 66, any other suitable clause, or any combination of suitable clauses, wherein X represents a linker.
196. A dimer protein consisting essentially of an amino acid sequence of SEQ ID NO: 67, any other suitable clause, or any combination of suitable clauses, wherein X represents a linker.
197. A dimer protein consisting essentially of an amino acid sequence of SEQ ID NO: 68, any other suitable clause, or any combination of suitable clauses, wherein X represents a linker.
198. A dimer protein consisting essentially of an amino acid sequence of SEQ ID NO: 69, any other suitable clause, or any combination of suitable clauses, wherein X represents a linker.
199. A dimer protein consisting essentially of an amino acid sequence of SEQ ID NO: 70, any other suitable clause, or any combination of suitable clauses, wherein X represents a linker.
200. A dimer protein consisting essentially of an amino acid sequence of SEQ ID NO: 71, any other suitable clause, or any combination of suitable clauses, wherein X represents a linker.
201. A dimer protein consisting of an amino acid sequence of SEQ ID NO: 51, any other suitable clause, or any combination of suitable clauses, wherein X represents a linker.
202. A dimer protein consisting of an amino acid sequence of SEQ ID NO: 52, any other suitable clause, or any combination of suitable clauses, wherein X represents a linker.
203. A dimer protein consisting of an amino acid sequence of SEQ ID NO: 53, any other suitable clause, or any combination of suitable clauses, wherein X represents a linker.
204. A dimer protein consisting of an amino acid sequence of SEQ ID NO: 54, any other suitable clause, or any combination of suitable clauses, wherein X represents a linker.
205. A dimer protein consisting of an amino acid sequence of SEQ ID NO: 55, any other suitable clause, or any combination of suitable clauses, wherein X represents a linker.
206. A dimer protein consisting of an amino acid sequence of SEQ ID NO: 56, any other suitable clause, or any combination of suitable clauses, wherein X represents a linker.
207. A dimer protein consisting of an amino acid sequence of SEQ ID NO: 57, any other suitable clause, or any combination of suitable clauses, wherein X represents a linker.
208. A dimer protein consisting of an amino acid sequence of SEQ ID NO: 58, any other suitable clause, or any combination of suitable clauses, wherein X represents a linker.
209. A dimer protein consisting of an amino acid sequence of SEQ ID NO: 59, any other suitable clause, or any combination of suitable clauses, wherein X represents a linker.
210. A dimer protein consisting of an amino acid sequence of SEQ ID NO: 60, any other suitable clause, or any combination of suitable clauses, wherein X represents a linker.
211. A dimer protein consisting of an amino acid sequence of SEQ ID NO: 61, any other suitable clause, or any combination of suitable clauses, wherein X represents a linker.
212. A dimer protein consisting of an amino acid sequence of SEQ ID NO: 62, any other suitable clause, or any combination of suitable clauses, wherein X represents a linker.
213. A dimer protein consisting of an amino acid sequence of SEQ ID NO: 63, any other suitable clause, or any combination of suitable clauses, wherein X represents a linker.
214. A dimer protein consisting of an amino acid sequence of SEQ ID NO: 64, any other suitable clause, or any combination of suitable clauses, wherein X represents a linker.
215. A dimer protein consisting of an amino acid sequence of SEQ ID NO: 65, any other suitable clause, or any combination of suitable clauses, wherein X represents a linker.
216. A dimer protein consisting of an amino acid sequence of SEQ ID NO: 66, any other suitable clause, or any combination of suitable clauses, wherein X represents a linker.
217. A dimer protein consisting of an amino acid sequence of SEQ ID NO: 67, any other suitable clause, or any combination of suitable clauses, wherein X represents a linker.
218. A dimer protein consisting of an amino acid sequence of SEQ ID NO: 68, any other suitable clause, or any combination of suitable clauses, wherein X represents a linker.
219. A dimer protein consisting of an amino acid sequence of SEQ ID NO: 69, any other suitable clause, or any combination of suitable clauses, wherein X represents a linker.
220. A dimer protein consisting of an amino acid sequence of SEQ ID NO: 70, any other suitable clause, or any combination of suitable clauses, wherein X represents a linker.
221. A dimer protein consisting of an amino acid sequence of SEQ ID NO: 71, any other suitable clause, or any combination of suitable clauses, wherein X represents a linker.
222. The dimer protein of any one of clauses 155 to 221, any other suitable clause, or any combination of suitable clauses, wherein the linker is selected from the group consisting of (GS)n, (GGGGS) n. (PS)n, and (PT)n, wherein n represents any number between 2-100.
223. The dimer protein of any one of clauses 155 to 221, any other suitable clause, or any combination of suitable clauses, wherein the linker is (G₄S)x4.
224. The dimer protein of any one of clauses 155 to 221, any other suitable clause, or any combination of suitable clauses, wherein the linker comprises

(SEQ ID NO: 73)
SSSGGGGSEFGGGGSGGGGSGGGGSAS.

225. The dimer protein of any one of clauses 155 to 221, any other suitable clause, or any combination of suitable clauses, wherein the linker consists essentially of

(SEQ ID NO: 73)
SSSGGGGSEFGGGGSGGGGSGGGGSAS.

226. The dimer protein of any one of clauses 155 to 221, any other suitable clause, or any combination of suitable clauses, wherein the linker consists of

(SEQ ID NO: 73)
SSSGGGGSEFGGGGSGGGGSGGGGSAS.

227. A dimer protein comprising a first monomer protein and a second monomer protein.
228. The dimer protein of clause 227, any other suitable clause, or any combination of suitable clauses, wherein the first monomer protein is the monomer protein of any one of clauses 1 to 154.
229. The dimer protein of clause 227, any other suitable clause, or any combination of suitable clauses, wherein the second monomer protein is the monomer protein of any one of clauses 1 to 154.
230. The dimer protein of clause 227, any other suitable clause, or any combination of suitable clauses, wherein the first monomer protein is the monomer protein of any one of clauses 1 to 154 and the second monomer protein is the monomer protein of any one of clauses 1 to 154.
231. The dimer protein of any of clauses 227 to 230, any other suitable clause, or any combination of suitable clauses, wherein the first monomer protein and the second monomer protein are connected via a linker.
232. The dimer protein of clause 231, any other suitable clause, or any combination of suitable clauses, wherein the linker is selected from the group consisting of (GS)n, (GGGGS)n, (PS)n, and (PT)n, wherein n represents any number between 2-100.
233. The dimer protein of clause 231, any other suitable clause, or any combination of suitable clauses, wherein the linker is (G₄S)x4.
234. The dimer protein of any one of clauses 231 to 233, any other suitable clause, or any combination of suitable clauses, wherein the linker comprises

(SEQ ID NO: 73)
SSSGGGGSEFGGGGSGGGGSGGGGSAS.

235. The dimer protein of any one of clauses 231 to 233, any other suitable clause, or any combination of suitable clauses, wherein the linker consists essentially of

(SEQ ID NO: 73)
SSSGGGGSEFGGGGSGGGGSGGGGSAS.

236. The dimer protein of any one of clauses 231 to 233, any other suitable clause, or any combination of suitable clauses, wherein the linker consists of

(SEQ ID NO: 73)
SSSGGGGSEFGGGGSGGGGSGGGGSAS.

237. A trimer protein comprising a first monomer protein, a second monomer protein, and a third monomer protein.
238. The trimer protein of clause 237, any other suitable clause, or any combination of suitable clauses, wherein the first monomer protein is the monomer protein of any one of clauses 1 to 154.
239. The trimer protein of clause 237, any other suitable clause, or any combination of suitable clauses, wherein the second monomer protein is the monomer protein of any one of clauses 1 to 154.
240. The trimer protein of clause 237, any other suitable clause, or any combination of suitable clauses, wherein the third monomer protein is the monomer protein of any one of clauses 1 to 154.
241. The trimer protein of clause 237, any other suitable clause, or any combination of suitable clauses, wherein the first monomer protein is the monomer protein of any one of clauses 1 to 154, the second monomer protein is the monomer protein of any one of clauses 1 to 154, and the third monomer protein is the monomer protein of any one of clauses 1 to 154.
242. The trimer protein of any of clauses 237 to 241, any other suitable clause, or any combination of suitable clauses, wherein the first monomer protein and the second monomer protein are connected via a linker.
243. The trimer protein of clause 242, any other suitable clause, or any combination of suitable clauses, wherein the linker is selected from the group consisting of (GS)n, (GGGGS)n, (PS)n, and (PT)n, wherein n represents any number between 2-100.
244. The trimer protein of clause 242, any other suitable clause, or any combination of suitable clauses, wherein the linker is (G₄S)x4.
245. The trimer protein of any one of clauses 242 to 244, any other suitable clause, or any combination of suitable clauses, wherein the linker comprises

(SEQ ID NO: 73)
SSSGGGGSEFGGGGSGGGGSGGGGSAS.

246. The trimer protein of any one of clauses 242 to 244, any other suitable clause, or any combination of suitable clauses, wherein the linker consists essentially of SSSGGGGSEFGGGGSGGGGSGGGGSAS (SEQ ID NO: 73).
247. The trimer protein of any one of clauses 242 to 244, any other suitable clause, or any combination of suitable clauses, wherein the linker consists of

(SEQ ID NO: 73)
SSSGGGGSEFGGGGSGGGGSGGGGSAS.

248. The trimer protein of any of clauses 237 to 247, any other suitable clause, or any combination of suitable clauses, wherein the second monomer protein and the third monomer protein are connected via a second linker.
249. The trimer protein of clause 248, any other suitable clause, or any combination of suitable clauses, wherein the second linker is selected from the group consisting of (GS)n, (GGGGS)n, (PS)n, and (PT)n, wherein n represents any number between 2-100.
250. The trimer protein of clause 248, any other suitable clause, or any combination of suitable clauses, wherein the second linker is (G₄S)x4.
251. The trimer protein of any one of clauses 248 to 250, any other suitable clause, or any combination of suitable clauses, wherein the second linker comprises

(SEQ ID NO: 73)
SSSGGGGSEFGGGGSGGGGSGGGGSAS.

252. The trimer protein of any one of clauses 248 to 250, any other suitable clause, or any combination of suitable clauses, wherein the second linker consists essentially of

(SEQ ID NO: 73)
SSSGGGGSEFGGGGSGGGGSGGGGSAS.

253. The trimer protein of any one of clauses 248 to 250, any other suitable clause, or any combination of suitable clauses, wherein the second linker consists of

(SEQ ID NO: 73)
SSSGGGGSEFGGGGSGGGGSGGGGSAS.

254. A pharmaceutical composition comprising the monomer protein of any one of clauses 1 to 154.
255. The pharmaceutical composition of clause 254, any other suitable clause, or any combination of suitable clauses, wherein the pharmaceutical composition is a parenteral formulation.
256. The pharmaceutical composition of clause 254, any other suitable clause, or any combination of suitable clauses, wherein the parenteral formulation is selected from the group consisting of intravenous, intraarterial, intraperitoneal, intrathecal, intradermal, epidural, intracerebroventricular, intraurethral, intrasternal, intracranial, intratumoral, intramuscular and subcutaneous.
257. The pharmaceutical composition of clause 254, any other suitable clause, or any combination of suitable clauses, wherein the pharmaceutical composition comprises one or more pharmaceutically acceptable carriers.
258. The pharmaceutical composition of clause 254, any other suitable clause, or any combination of suitable clauses, wherein the pharmaceutical composition further comprises a second therapeutic agent.
259. The pharmaceutical composition of clause 254, any other suitable clause, or any combination of suitable clauses, wherein the pharmaceutical composition is formulated as a single dose.
260. The pharmaceutical composition of clause 254, any other suitable clause, or any combination of suitable clauses, wherein the pharmaceutical composition is formulated as a single unit dose.
261. A pharmaceutical composition comprising the dimer protein of any one of clauses 155 to 236.
262. The pharmaceutical composition of clause 261, any other suitable clause, or any combination of suitable clauses, wherein the pharmaceutical composition is a parenteral formulation.
263. The pharmaceutical composition of clause 261, any other suitable clause, or any combination of suitable clauses, wherein the parenteral formulation is selected from the group consisting of intravenous, intraarterial, intraperitoneal, intrathecal, intradermal, epidural, intracerebroventricular, intraurethral, intrasternal, intracranial, intratumoral, intramuscular and subcutaneous.
264. The pharmaceutical composition of clause 261, any other suitable clause, or any combination of suitable clauses, wherein the pharmaceutical composition comprises one or more pharmaceutically acceptable carriers.
265. The pharmaceutical composition of clause 261, any other suitable clause, or any combination of suitable clauses, wherein the pharmaceutical composition further comprises a second therapeutic agent.
266. The pharmaceutical composition of clause 261, any other suitable clause, or any combination of suitable clauses, wherein the pharmaceutical composition is formulated as a single dose.
267. The pharmaceutical composition of clause 261, any other suitable clause, or any combination of suitable clauses, wherein the pharmaceutical composition is formulated as a single unit dose.
268. A pharmaceutical composition comprising the trimer protein of any one of clauses 237 to 253.
269. The pharmaceutical composition of clause 268, any other suitable clause, or any combination of suitable clauses, wherein the pharmaceutical composition is a parenteral formulation.
270. The pharmaceutical composition of clause 268, any other suitable clause, or any combination of suitable clauses, wherein the parenteral formulation is selected from the group consisting of intravenous, intraarterial, intraperitoneal, intrathecal, intradermal, epidural, intracerebroventricular, intraurethral, intrasternal, intracranial, intratumoral, intramuscular and subcutaneous.
271. The pharmaceutical composition of clause 268, any other suitable clause, or any combination of suitable clauses, wherein the pharmaceutical composition comprises one or more pharmaceutically acceptable carriers.
272. The pharmaceutical composition of clause 268, any other suitable clause, or any combination of suitable clauses, wherein the pharmaceutical composition further comprises a second therapeutic agent.
273. The pharmaceutical composition of clause 268, any other suitable clause, or any combination of suitable clauses, wherein the pharmaceutical composition is formulated as a single dose.
274. The pharmaceutical composition of clause 268, any other suitable clause, or any combination of suitable clauses, wherein the pharmaceutical composition is formulated as a single unit dose.
275. A method of treating a Shiga toxin-producing Escherichia coli (STEC) infection in a patient, said method comprising the step of administering a therapeutically effective amount of the pharmaceutical composition of any one of clauses 254 to 274 to the patient.
276. The method of clause 275, any other suitable clause, or any combination of suitable clauses, wherein the pharmaceutical composition reduces one or more symptoms of the STEC infection in the patient.
277. The method of clause 276, any other suitable clause, or any combination of suitable clauses, wherein the one or more symptoms comprise a gastrointestinal symptom.
278. The method of clause 276, any other suitable clause, or any combination of suitable clauses, wherein the one or more symptoms comprise diarrhea.
279. The method of clause 276, any other suitable clause, or any combination of suitable clauses, wherein the one or more symptoms comprise bloody diarrhea.
280. The method of clause 276, any other suitable clause, or any combination of suitable clauses, wherein the one or more symptoms comprise stomach cramps.
281. The method of clause 276, any other suitable clause, or any combination of suitable clauses, wherein the one or more symptoms comprise vomiting.
282. The method of clause 276, any other suitable clause, or any combination of suitable clauses, wherein the one or more symptoms comprise hemolytic uremic syndrome (HUS). 283. The method of clause 275, any other suitable clause, or any combination of suitable clauses, wherein the method further comprises administration of an antibiotic to the patient.
284. The method of clause 275, any other suitable clause, or any combination of suitable clauses, wherein the method further comprises administration of intravenous fluids to the patient.
285. The method of clause 275, any other suitable clause, or any combination of suitable clauses, wherein the administration is a parenteral administration.
286. The method of clause 285, any other suitable clause, or any combination of suitable clauses, wherein the parenteral administration is selected from the group consisting of intravenous, intraarterial, intraperitoneal, intrathecal, intradermal, epidural, intracerebroventricular, intraurethral, intrasternal, intracranial, intratumoral, intramuscular and subcutaneous.
287. The method of clause 285, any other suitable clause, or any combination of suitable clauses, wherein the parenteral administration is intravenous.
288. The method of clause 285, any other suitable clause, or any combination of suitable clauses, wherein the parenteral administration is intraarterial.
289. The method of clause 285, any other suitable clause, or any combination of suitable clauses, wherein the parenteral administration is intraperitoneal.
290. The method of clause 285, any other suitable clause, or any combination of suitable clauses, wherein the parenteral administration is intrathecal.
291. The method of clause 285, any other suitable clause, or any combination of suitable clauses, wherein the parenteral administration is intradermal.
292. The method of clause 285, any other suitable clause, or any combination of suitable clauses, wherein the parenteral administration is epidural.
293. The method of clause 285, any other suitable clause, or any combination of suitable clauses, wherein the parenteral administration is intracerebroventricular.
294. The method of clause 285, any other suitable clause, or any combination of suitable clauses, wherein the parenteral administration is intraurethral.
295. The method of clause 285, any other suitable clause, or any combination of suitable clauses, wherein the parenteral administration is intrasternal.
296. The method of clause 285, any other suitable clause, or any combination of suitable clauses, wherein the parenteral administration is intracranial.
297. The method of clause 285, any other suitable clause, or any combination of suitable clauses, wherein the parenteral administration is intramuscular.
298. The method of clause 285, any other suitable clause, or any combination of suitable clauses, wherein the parenteral administration is subcutaneous.
299. A method of treating a hemolytic uremic syndrome (HUS) in a patient, said method comprising the step of administering a therapeutically effective amount of the pharmaceutical composition of any one of clauses 254 to 274 to the patient.
300. The method of clause 299, any other suitable clause, or any combination of suitable clauses, wherein the patient is a child patient.
301. The method of clause 299, any other suitable clause, or any combination of suitable clauses, wherein the patient is an elderly patient.
302. The method of clause 299, any other suitable clause, or any combination of suitable clauses, wherein the method further comprises administration of an antibiotic to the patient.
303. The method of clause 299, any other suitable clause, or any combination of suitable clauses, wherein the method further comprises administration of intravenous fluids to the patient.
304. The method of clause 299, any other suitable clause, or any combination of suitable clauses, wherein the method further comprises administration of blood transfusion to the patient.
305. The method of clause 299, any other suitable clause, or any combination of suitable clauses, wherein the method further comprises administration of plasma exchange to the patient.
306. The method of clause 299, any other suitable clause, or any combination of suitable clauses, wherein the method further comprises administration of kidney dialysis to the patient.
307. The method of clause 299, any other suitable clause, or any combination of suitable clauses, wherein the administration is a parenteral administration.
308. The method of clause 307, any other suitable clause, or any combination of suitable clauses, wherein the parenteral administration is selected from the group consisting of intrathecal, intravenous, intraarterial, intraperitoneal, intradermal, epidural, intracerebroventricular, intraurethral, intrasternal, intracranial, intratumoral, intramuscular and subcutaneous.
309. The method of clause 307, any other suitable clause, or any combination of suitable clauses, wherein the parenteral administration is intravenous.
310. The method of clause 307, any other suitable clause, or any combination of suitable clauses, wherein the parenteral administration is intraarterial.
311. The method of clause 307, any other suitable clause, or any combination of suitable clauses, wherein the parenteral administration is intraperitoneal.
312. The method of clause 307, any other suitable clause, or any combination of suitable clauses, wherein the parenteral administration is intrathecal.
313. The method of clause 307, any other suitable clause, or any combination of suitable clauses, wherein the parenteral administration is intradermal.
314. The method of clause 307, any other suitable clause, or any combination of suitable clauses, wherein the parenteral administration is epidural.
315. The method of clause 307, any other suitable clause, or any combination of suitable clauses, wherein the parenteral administration is intracerebroventricular.
316. The method of clause 307, any other suitable clause, or any combination of suitable clauses, wherein the parenteral administration is intraurethral.
317. The method of clause 307, any other suitable clause, or any combination of suitable clauses, wherein the parenteral administration is intrasternal.
318. The method of clause 307, any other suitable clause, or any combination of suitable clauses, wherein the parenteral administration is intracranial.
319. The method of clause 307, any other suitable clause, or any combination of suitable clauses, wherein the parenteral administration is intramuscular.
320. The method of clause 307, any other suitable clause, or any combination of suitable clauses, wherein the parenteral administration is subcutaneous.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

### Example 1

### Exemplary Experimental Procedures

The instant example provides exemplary materials and methods utilized in Examples 2 to 6 as described herein.

### Protein Expression and Purification.

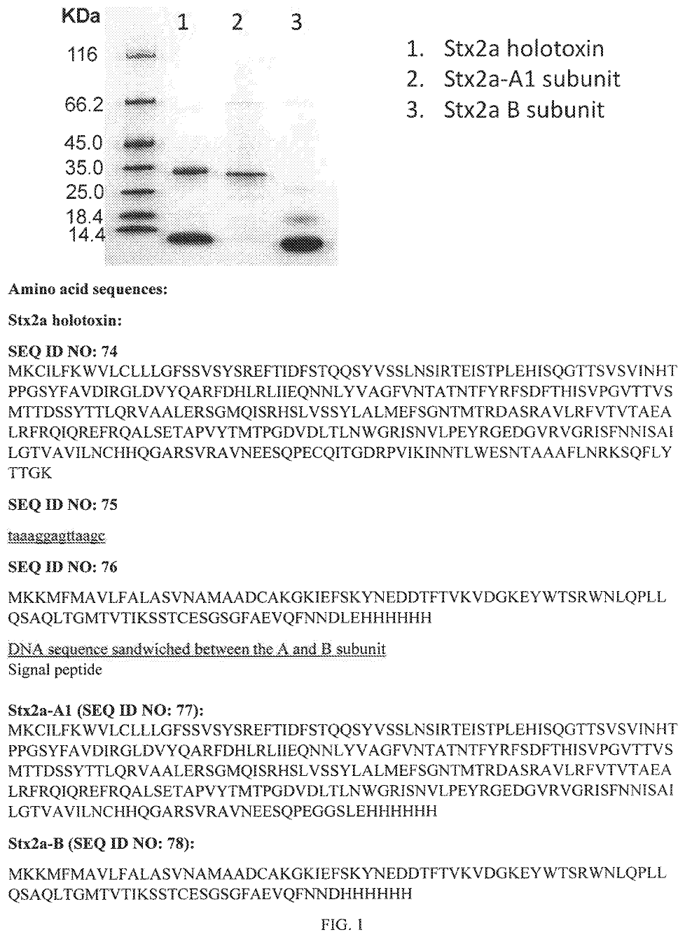

Stx2a, Stx2a-A1 and Stx2a-B were recombinantly expressed in E. coli BL21 (DE3) cells and purified via the use of a nickel-nitrilotriacetic acid (Ni-NTA) column. The holotoxin Stx2a was concentrated and buffer exchanged into phosphate-buffered saline (1×PBS, pH 7.4) using Amicon ultrafiltration unit (MWCO 50 kDa). Protein purity was confirmed using SDS-PAGE. The concentration of purified protein was determined by BCA assay (Thermo Scientific Pierce BCA Protein Assay, Fisher catalog no. PI23227). The purified protein was stored at −20° C. in 50% glycerol. (FIG. 1). Purified Stx2c was obtained from BEI Resources (Cat #NR-13422).

### DARPin Library Construction.

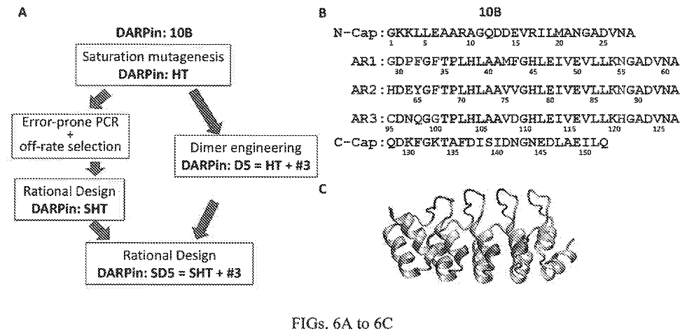

An in-house prepared DARPin library with ˜2×109 variants was used in the initial phage panning. For the saturation mutagenesis, the selected residues (FIG. 6B) were replaced with the NNK codon. The resulting library was cloned into pET28a vector and transformed into B121 (DE3) cells for protein expression and functional screening. For the off-rate selection library, error-prone PCR was used to introduce random mutations to selected monomer DARPins (i.e. 10B, HT, RI, #1, #3, #9, #12, #16 and #20). The mutation rate was determined to be 3.7 nucleotide changes per gene based on the sequencing result. This library was displayed on M13 phage as an N-terminal fusion to gIII and used in phage panning. The dimeric DARPin library utilized a (G₄S)x4 linker was used to connect the two DARPins.

### Phage Panning

Holotoxin Stx2a was biotinylated using EZ-Link-Sulfo NHS-LC biotin (Pierce) and used as the target in phage panning. DARPin variants from the 3^{rd} round of phage panning was cloned into pET28a vector (containing an N-terminal His tag and a Myc tag). A total 752 different colonies of E. coli B121 (DE3) cells transformed with the enriched DARPin library were picked and grown in 96-well plates.

For off-rate selection, the phage library (˜1012 phage particles) was first incubated with biotinylated Stx2a (1 nM) in 1 mL of PBS overnight at 4° C. The next day, a 500-fold molar excess of non-biotinylated Stx2a (500 nM) was added, and this mixture was incubated with slow shaking for 4, 16 and 140 hours at room temperature for round 1, 2, and 3, respectively. Phage particles that remain bound to the biotinylated toxin should be enriched for variants with a slower off-rate and were recovered using Dynabeads MyOne streptavidin-coated magnetic beads (Thermofisher Scientific). To ensure the enrichment of DARPin molecules able to remain associated with Stx2a upon translocation into the acidic endosome, the beads were thoroughly washed in both regular and acidic PBS+0.1% Tween-20 (PBST) buffer (pH 4.5, buffered with 10 mM sodium citrate) before the elution of the bound phage.

### Functional Screening

The expressed DARPins were semi-purified via lysozyme and heat treatment, and the toxin neutralization potency of each selected DARPin was evaluated using the Vero or Vero-E6 cell toxin challenging assay. For the initial monomer DARPin screening, Vero cells were seeded the night before in 96-well plate (1.5×10³ cells/well, 100 μL/well) in complete growth medium supplemented with 10% FBS and 1×anti-antibiotic antimycotic (Life Technologies catalog no. 15240062). The next day, the soluble cell lysate (20 μL/well) and Stx2a (80 μL, final 10 μg/mL) were added to the Vero cells (total 200 μL/well). The plates were incubated at 37° C. and 5% CO₂ for 72 hours and the cell viability was quantified using the CellTiter-Glo reagent (Promega) and normalized to that of naïve Vero cells.

For dimer library and the off-rate library screening, Vero E6 cells were used instead. A mixture of Vero E6 cells (100 μL, 1.5×10³ cells/well), the soluble lysate (20 μL/well) and diluted Stx2a (80 μL, final 10 μg/mL or 0.1 μM) were seeded simultaneously in 96-well plates. The cell viability was quantified as described above.

### Protein Purification

Candidate DARPin clones were purified by standard metal chelation chromatography as previously described. For the in vivo experiment, the eluted protein was first buffer-exchanged into DPBS (Fisher, cat SH30028FS) and passed through two Pierce™ High Capacity Endotoxin Removal Spin Columns (Fisher, Cat #P188271) to remove the endotoxin. The endotoxin level in the final purified protein was determined to be 14 EU/mL using Pierce™ Chromogenic Endotoxin Quant Kit (Thermo Scientific, Cat #A39553).

### Vero E6 Toxin Challenging Assay

A mixture containing Vero E6 cells (1.5×10⁴ cells/well), purified Stx2a (final 5 μg/mL or 57 fM) and diluted DARPin was seeded in 96-well plates (200 μL/well). The plates were incubated at 37° C./5% CO₂ for 72 hours, and the cell viability was quantified using the CellTiterGlo reagent (Promega). Relative viability is calculated using the equation below:

$$Relative Viability = \frac{{R}_{( cells + DARPin + toxin )} - {R}_{( toxin only )}}{{R}_{( cells only )} - {R}_{( toxin only )}}$$

### In Vitro Catalytic Activity Assay

Low-profile PCR tubes (200 μL capacity) were first blocked with 150 μL PBS+0.2% BSA at room temperature for 1 hour and then washed twice with DPBS (Fisher, Cat #SH30028FS). Purified Stx2a-A1 (37.5 μM in DPBS) was incubated with equal volume of DARPin (150 μM in DPBS) or DPBS alone at 37° C. for 20 minutes to allow DARPin to form complexes with the toxin. Next, 1.6 μL of this mixture was added to 1×NEBExpress Cell-free E. coli Protein Synthesis System (NEB, Cat #E5360S) containing 3 μL of S30 extract, 6 μL of Protein Synthesis Buffer, 0.2 μL T7 RNA polymerase and 0.2 μL RNase Inhibitor, Murine. The reaction was incubated at 37° C. for 30 minutes during which time the toxin Stx2a-A1 is able to inactivate the ribosomes. Next, a plasmid DNA encoding a reporter eGFP gene under a T7 promoter (75 ng) was added to the reaction to initiate RNA synthesis and protein translation. The reaction was carried out at 37° C. for 6 hours followed by overnight incubation at 4° C. to ensure complete folding of eGFP. Forty-five μL of ddH₂O was then added to each tube and mixture was transferred to a black 384 well plate (25 L/well) for the quantification of fluorescence intensity using Cytation 5 plate reader (Agilent). The positive controls contained no toxin and the negative controls contained toxin but no DARPin.

### ELISA

The ELISA experiments were carried out with Stx2a, Stx2a-A1 or Stx2a-B used to coat the wells of MaxiSorp immunoplates (Fisher, Nunc MaxiSorp ELISA plates, Cat #50-712-278) at 4 μg/mL. All DARPins contain a Myc tag at the N-terminus. For affinity measurement, well-bound DARPin molecules were detected using mouse anti-c-Myc antibody (Invitrogen Cat #13-2500) and horseradish peroxidase (HRP)-conjugated goat anti-mouse antibody (Jackson Immuno Research, Cat #115-035-146).

### Affinity Measurement

The affinity measurement was performed on a Fortebio BLItz instrument (Sartorius, Goettingen, Germany) using the Streptavidin sensors (Sartorius #18-5019). Purified holotoxin Stx2a (final 1 mg/mL) was first biotinylated using EZ-Link Sulfo-NHS-LC-Biotin (Pierce) at 1:10 molar ratio in PBS at 4° C. for 16 hours and then passed through a Zeba spin desalting column (7K, MWCO, ThermoFisher) to remove the excess biotin molecules. Biotinylated Stx2a (0.37 μM) were captured onto the sensor. PBS with 0.1% BSA (Fisher #BP9706100) was used as the buffer for protein dilution, sensor equilibration and washing. Subsequently, the biosensors were dipped into serially diluted DARPin proteins. Every measurement run included 30 seconds of equilibration, 60 seconds of Stx2a loading, 30 seconds of washing, 60 seconds of DARPin association and 60 seconds of dissociation. BLItz pro 1.3 software was used for binding detection and analysis. K_{D} values were the average of at least three K_{D} values calculated using the local fitting method.

### Cryo-EM Sample Preparation

Stx2a and #3 DARPin were mixed at 1:1 molar ratio (with the final concentration of the complex at 800 nM) and incubated in PBS buffer at pH 7.4 for 30 minutes at room temperature; 3 μL of the complex was applied to C-flat 2/1 holey carbon film 300 mesh grids at 20° C. with 100% relative humidity and vitrified using a Vitrobot (Mark III, FEI Company, the Netherlands).

### Cryo-EM Data Collection

The complex of Stx2a and #3 DARPin was imaged under the Thermo-Fisher Talos Arctica electron microscope (Thermo-Fisher), with field emission gun operated at 200 kV. The microscope is equipped with a Gatan K2 summit direct detection camera (Gatan, Pleasanton, CA); 3,500 micrographs were collected in electron-counting mode at 1.07 Å/pix. The beam intensity was adjusted to 5 e⁻/Å²/s on the camera. A 33-frame movie stack was collected for each picture, with 0.2 seconds per frame, for a total exposure time of 6.6 seconds.

### Image Processing

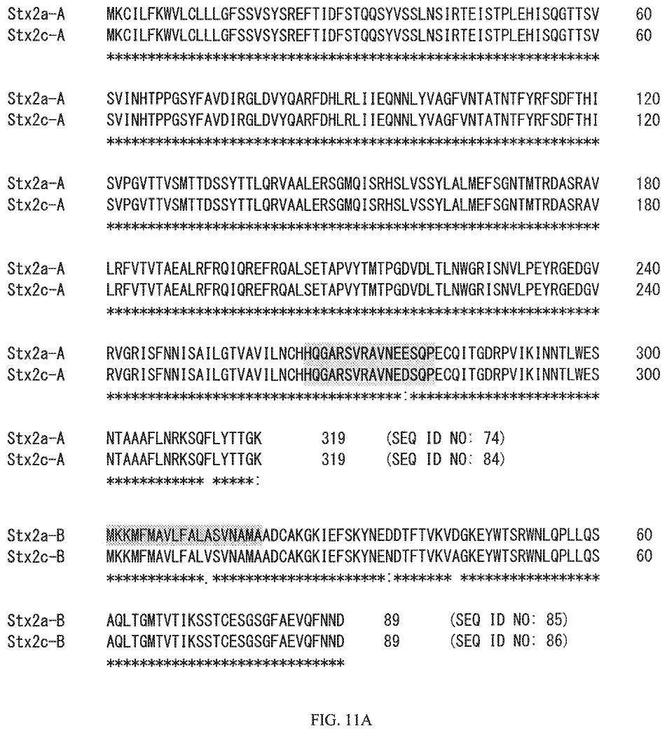

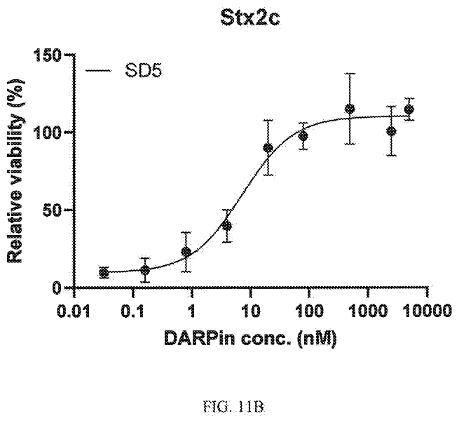

The collected movies for the complex of Stx2a and #3 DARPin were processed by cryoSPARC, (FIG. 11A). These stacks were aligned using Motioncorrection 2 and estimated contrast transfer functions using patch CTF estimation. 1606 micrographs with strong power spectra were used to pick particles using blob picker. After generating 2D averages from particles picked by blob picker, the particles were picked again by template picker, yielding 797,974 particles. These particles were subjected to 2D classification for particle cleaning, generating 251,399 particles for ab initio reconstruction and heterogenous refinement into two classes. The reconstructed two density maps are the apo-state Stx2a and the bound-state Stx2a complex. Further refinement was done on each of the state. The overall resolution was assessed using the gold-standard criterion of Fourier Shell Correlation, with a cutoff at 0.143, between 2 half maps from 2 independent half-sets of data.

### Model Building

The homology model of DARPin #3 generated by SWISS-MODEL and the initial model for Stx2a (pdb: 1r4p) were roughly docked into the density map of Stx2a and DARPin complex. The position of Stx2a was clear docked since the subunit a and subunit b can be distinguished easily. The docking of DARPin was obtained by hitting the best fitting score calculated in Chimera. The fitted model was further refined in phenix to achieve better geometry. The apo state Stx2a model was refined using the apo state Stx2a density map described above. To compare the steric positions of Gb₃ and DARPin, The Gb₃ positions were obtained from solved crystal structure of StxB and Gb₃ complex (pdb: 1bos) and superimposed on the refined Stx2a-DARPin structure.

The homology model of DARPin SHT was generated by SWISS-MODEL and templated on a DARPin molecule (pdb: 1mj0). The protein structures were visualized using the VMD software.

### Data Availability

Models are available from the Protein DataBank with accession numbers: 7UJJ for the Stx2-DARPin complex; Density maps are available from EM DataBank with accession numbers: EMD-26563 and EMD-26565 for the Stx2-DARPin complex and apo state Stx2, respectively. All other relevant data will be provided upon request.

### Stx Toxin Challenge in Mice

Four- to five-week-old female Swiss Webster mice (average 25 g) were inoculated intraperitoneally with either Stx2a (60 ng) mixed with PBS (100 μL), or Stx2a (60 ng) mixed with DARPin DA1-SD5 (250 μg or 125 μg) in PBS (100 μL) immediately before injection. This dose of toxin corresponds to 1.25× the minimum lethal dose (MLD) determined previously by us. Mice were monitored for changes in body weight, neurologic signs including change in demeanor, activity and responsiveness to stimuli. When mice exhibited progressive clinical signs, became less responsive to stimuli, or lost 20% of bodyweight, they were humanely euthanized.

### Example 2

### Selection of Monomeric Stx2a-Neutralizing DARPins

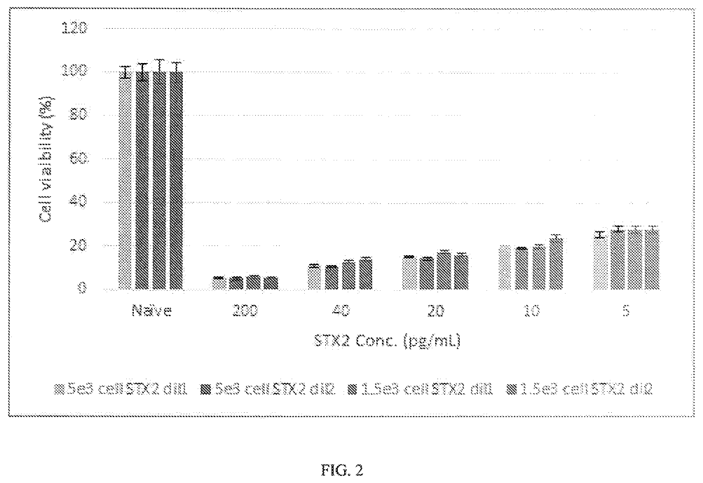

In the instant example, a strategy to purify holotoxin Stx2a was developed. A 6×His tag was fused to the C-terminus of the B subunit and the holotoxin was expressed in the periplasmic region of E. coli and purified by one-step affinity purification. To remove all monomeric A-(35 kDa) and B-subunit (7 kDa), the purified protein was concentrated via ultrafiltration (MWCO 50 kDa) to recover only the assembled holotoxin (AB₅, 70 kDa, FIG. 1). The purified Stx2a holotoxin is highly toxic to Vero E6 cells with an estimated IC₅₀ of <5 μg/mL, confirming the correct folding of the holotoxin (FIG. 2). Increasing the toxin concentration from 10 to 200 μg/mL resulted in moderately reduced cell viability from ˜20% to ˜5%. Without being bound by any theory, the assay condition, in which the toxin was added to low density of Vero cells at the time of cell seeding, may be in part responsible for this seemingly flat toxicity curve.

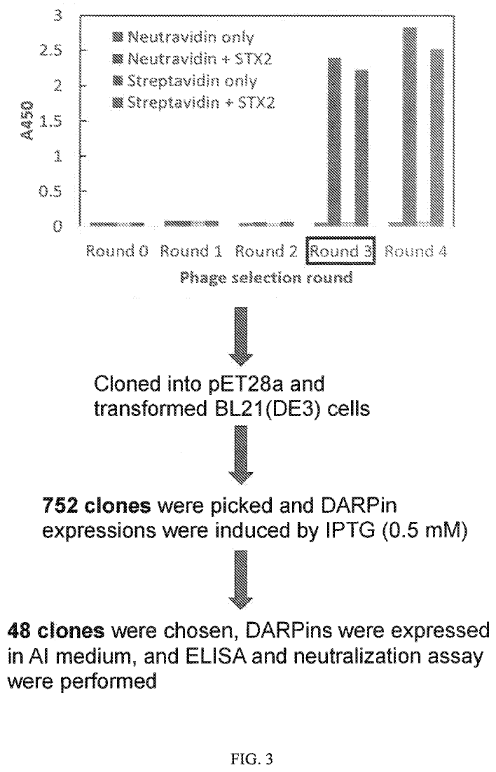

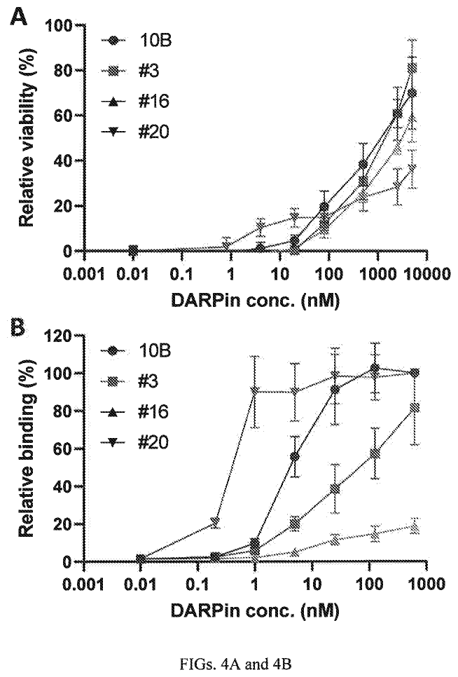

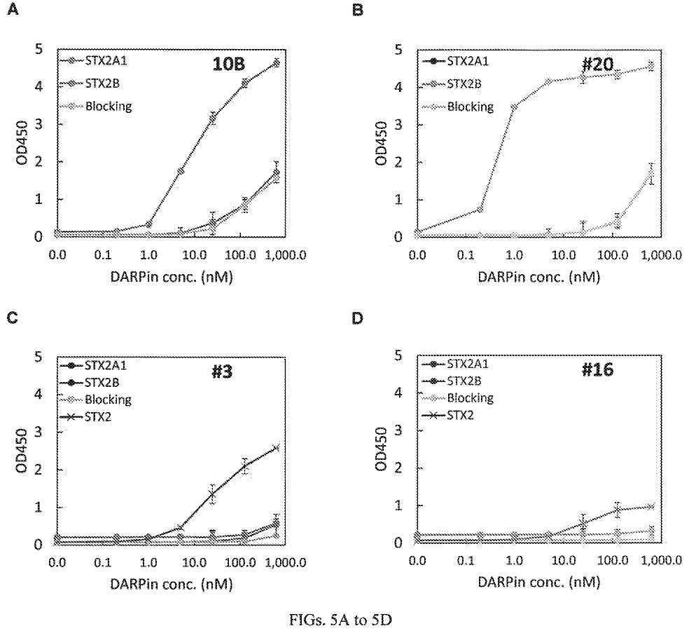

Using a DARPin library with ˜2×109 diversity, 4 rounds of phage panning against biotinylated holotoxin Stx2a were carried out. The enriched phage pool from the 3^{rd} round of selection, which showed significantly enhanced ability to bind Stx2a (FIG. 3), was cloned into the pET28a vector and transformed into E. coli BL21 (DE3) cells for high-level DARPin expression. 752 colonies were picked and grown in 96-well plates, and cell lysates were subjected to functional screening for those able to rescue Vero E6 cells from Stx2a toxicity. Forty-eight candidate clones were selected and sequenced, yielding 21 unique clones. These clones were individually expressed and purified via affinity chromatography and their toxin neutralization and binding abilities were evaluated using the Vero-E6 toxin challenge assay and ELISA, respectively. Among these, 19 clones exhibited measurable toxin neutralization activity. The four best clones (#3, 10B, #16 and #20) neutralized Stx2a (5 μg/mL or 57 fM) with EC₅₀>1 μM (FIG. 4A), which, without being bound by any theory, may be inadequate for therapeutic applications. The ability of these DARPin molecules to bind Stx2a does not appear to correlate with the neutralization potency (FIG. 4B). The subunits targeted by these DARPins using purified A1- and B-subunit of Stx2a (FIG. 1) was further investigated. DARPin 10B and #20 bind strongly to the A1 subunit while DARPin #3 and #16 failed to bind either of the subunits individually despite their apparent abilities to bind the holotoxin (FIGS. 5A-5D), indicating that these two DARPins, without being bound by any theory, may target a conformational epitope(s) present only on the AB₅ holotoxin.

### Example 3

### Systematic Affinity Maturation

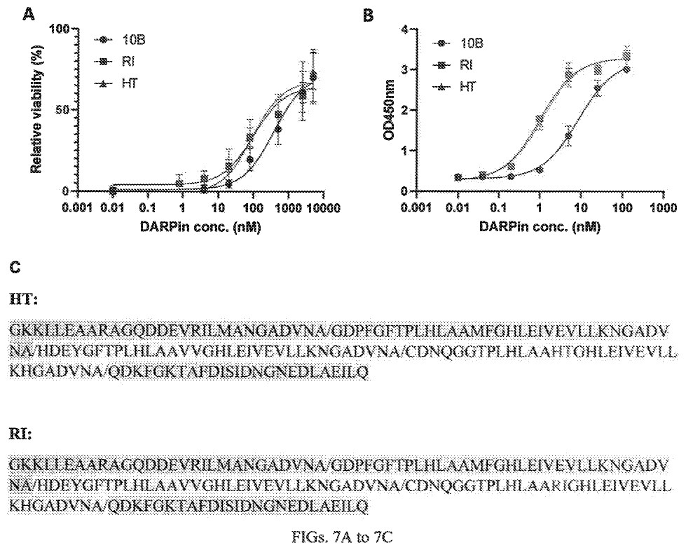

In the instant example, systematic affinity maturation was explored. The high toxin neutralization activity, indicating the association of an important epitope on the toxin, and medium toxin binding affinity of DARPin 10B prompted the selection for affinity maturation. An overview of the systematic affinity maturation strategy is shown in FIG. 6A. Saturation mutagenesis of residues in direct contact with the toxin was first performed. To maximize the diversity while maintaining a manageable library size, six saturation mutagenesis libraries were constructed with each library including a consecutive dipeptide randomized to all 20 amino acids (FIG. 6B, red residues). A total of 4800 colonies (800 per library) were screened using the Vero E6 toxin challenge assay, yielding 30 candidates with improved toxin-neutralization activity. These candidates were sequenced, revealing 19 unique clones. Interestingly, all these clones contain mutations at positions 108-109. The best DARPins, HT and RI (denoting the amino acids at positions 108, 109), showed ˜4-fold improved toxin neutralization activity in the Vero E6 toxin challenge assay and ˜8-fold improved toxin-binding ability in ELISA (FIGS. 7A, 7B, 7C).

Among the 11 mutations acquired by SHT, only two (108-109) are directly engaged in target binding. Residues 108-109 are located on the α-helix in AR3 and were originally occupied by Val-Asp in DARPin 10B. Through saturation mutagenesis, DARPin HT and RI whose residues at these positions were mutated to His-Thr and Arg-Ile, respectively, were obtained. Both HT and RI showed similarly improved binding affinity toward Stx2a FIG. 7B), indicating an important role of these residues in target interaction. However, the replacement of hydrophobic Val and negatively charged Asp in DARPin 10B with nucleophilic Thr and positively charged His in HT, and negatively charged Arg and hydrophobic Iso in RI, is not anticipated and their contribution is unclear without further structural studies. Without being bound by any theory, the remaining nine mutations in SHT may not directly participate in target interaction but subtly influence the binding interface. These mutations further increased the toxin neutralization potency of SHT by >220-fold compared to HT.

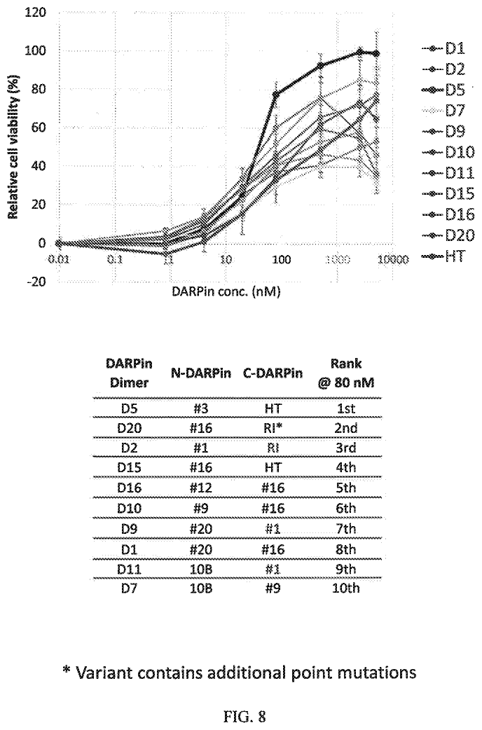

To further improve the toxin-neutralization potency through an avidity effect, a dimer library screening in which the 19 different first-generation DARPin monomers plus HT and RI were randomly linked together via a flexible (G₄S) 4 linker (expected to span ˜55 Å) was carried out. The theoretical library size is 441 (21×21). A total of 768 individual clones were screened using the Vero-E6 toxin challenge assay with 5 μg/mL Stx2a and 20 candidates with enhanced neutralization activity were identified. Sequencing of these clones revealed 10 unique combinations. These dimers were purified by one-step metal chelation chromatography and their toxin neutralization activity was evaluated. All dimers exhibit significantly enhanced neutralization potency compared to the best monomer HT (FIG. 8). The dimer D5 comprising DARPin #3 and HT neutralized Stx2a with EC₅₀ of 38.3 nM.

Concurrently, further reduction of the off-rate of the monomeric DARPins (FIG. 6A) was sought. For therapeutic applications, a critical factor limiting the efficacy of a binding protein (e.g. antibody) may be its retention time on the target. Selection of variants with slower off-rate using competitive off-rate selection has proven to be a powerful method for improving affinity. Error-prone PCR was used to generate randomized libraries of DARPin monomers. After three rounds of off-rate selection, a fourth round of phage panning was carried out using biotinylated Stx2a (2 nM) under standard conditions.

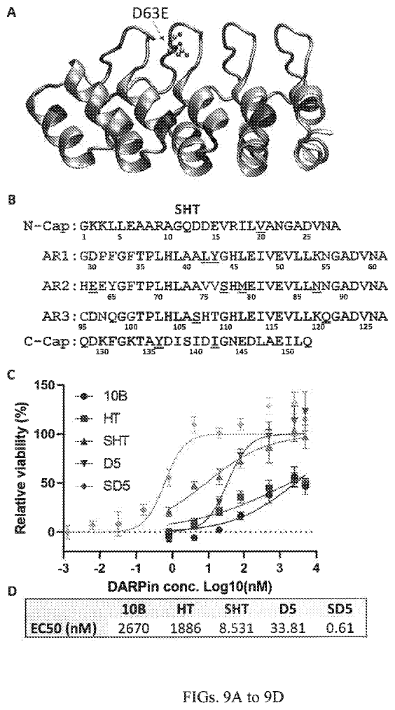

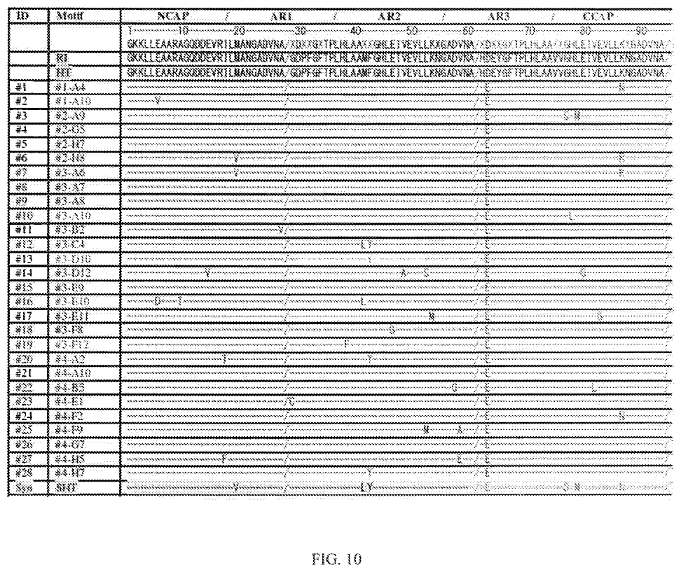

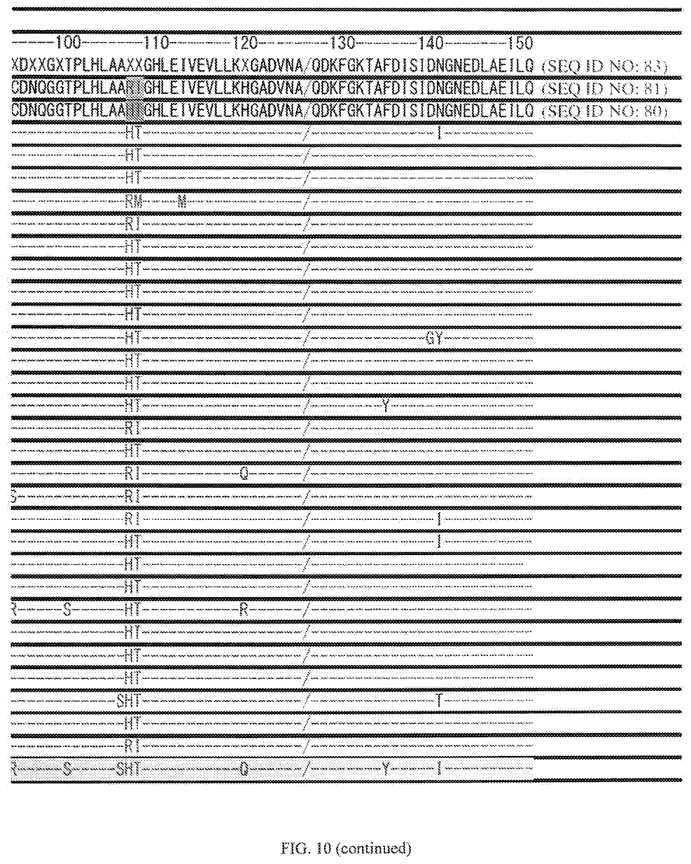

The enriched 4th-round selected error-prone PCR phage pool was cloned into pET28a for DARPin expression and the E. coli lysates were subjected to screening for Stx2a binding. A total of 400 colonies were screened yielding 28 unique clones with enhanced Stx2a binding relative to clone HT. Remarkably, all of these clones contained the D63E mutation (FIG. 10), pointing to an important role of this residue in Stx2a binding. Mutation D63E is located on AR2 near the tip of a binding loop (FIG. 9A) and is sandwiched between target binding residues at position 62, 64 and 65 that are randomized in the original DARPin library (FIG. 9B). His62 and Glu64 are both deemed important for target binding and were randomized in the original DARPin library. Without being bound by any theory, residue at position 63 was believed to be not directly involved in target binding and is occupied by Asp in the parental DARPin structure. Although both Asp and Glu are negatively charged, the side chain of glutamic acid has one additional carbon bond and may thus enable the formation of a more favorable interaction with the target protein. Twenty-one clones are derived from DARPin HT, and 7 are from DARPin RI, likely due to the stronger neutralization activity of these parental DARPins (FIG. 10). Point mutations from variants derived from DARPin HT were rationally combined based on their abundance and proximity to the target binding residues to generate variant SHT with a total of 11 mutations compared to the parent clone HT (FIG. 9B). DARPin SHT is derived from DARPin 10B, which binds the Stx2a A-subunit (FIG. 5). DARPin SHT exhibits >100-fold enhanced Stx2a neutralization potency (EC₅₀ 8.5 nM) relative to HT (EC₅₀ 1.9 μM) (FIG. 9D). Particularly, the toxin-neutralization potency of DARPin SHT is >300-fold and >220-fold stronger than its grandparent DARPin 10B and parent DARPin HT, respectively (FIG. 9D).

Among all the natural ankyrin repeat proteins, this position is highly conserved and is usually occupied by Asp, Thr or Ala. Although the exact mode of action of D63E is unclear without additional structural information, the increased affinity contributed by this mutation points to an important role of this residue in target interaction.

Finally, the HT in the best dimer DARPin D5 was replaced with SHT to give rise to dimer SD5 (#3-SHT) (FIG. 6A). Accordingly, the constituents of DARPin SD5 are monomer DARPins #3 and SHT. DARPin SD5 neutralized Stx2a with EC₅₀ of 0.6 nM (FIG. 9D), which is 4,450-fold more potent than the best 1^{st} generation DARPin 10B (EC₅₀ 2.7 μM), a strong testament to the power of sequential protein engineering approach employed in this study. The potency of SD5 against Stx2c, another genotype commonly involved in HUS was also determined. Despite high sequence homology (98.7% identical), SD5 showed slightly reduced, albeit still potent, antitoxin activity toward Stx2c with EC₅₀ of 7.3 nM (FIGS. 11A, 11B).

### Example 4

### In Vitro Characterization of DARPin Molecules

In the instant example, in vitro characterization of DARPin molecules was explored. Since DARPin 10B binds the A-subunit of Stx2a, its ability to inhibit the toxin's catalytic activity was first evaluated. In the cell, the A subunit is proteolytically processed to form the catalytically active A1 fragment and the smaller A2 fragment that remains associated with the pentameric B subunit. The A1 fragment is recognized by the ER export mechanism and translocated to the cytosol where it inhibits protein synthesis by removing an adenosine from the 3′ region of the 28S rRNA, triggering apoptosis.

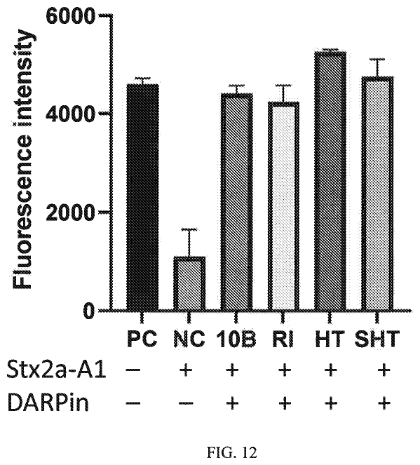

Stx2a-A1 fragment with a C-terminus 6×His tag was constructed, the protein was purified by metal chelation chromatography (FIG. 1), and the ability of DARPins to rescue ribosomes from intoxication by Stx2a-A1 was determined. As shown in FIG. 12, Stx2-A1 efficiently inhibited the protein synthesis, which can be rescued by DARPin 10B and its progenies. However, DARPin HT and SHT did not exhibit enhanced potency than 10B, which inhibits the enzymatic activity of the toxin. Without being bound by any theory, this may be due to the relatively high concentration of DARPin (i.e. 10 μM) used in this study, which may obscure the differences in target binding affinity between these DARPins.

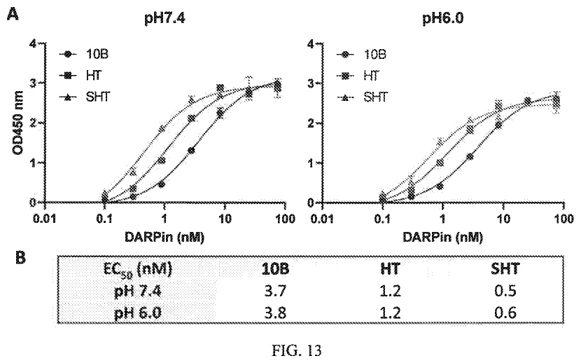

Next, the target binding ability of these DARPins using immobilized Stx2a-A1 by ELISA at both neutral (pH 7.4) and acidic (pH 6.0) conditions to mimic the environment in the extracellular space and within the endosome upon toxin internalization, respectively, were compared. The target binding ability gradually improved in the course of the engineering with DARPin SHT exhibiting 6-7-fold increased binding ability toward immobilized Stx2a-A1 than SHT (FIGS. 13A, 13B). In addition, the binding interaction between the DARPins and Stx2a-A1 appeared to saturate at concentrations >100 nM, consistent with the observed similar inhibitory activity in the above in vitro catalytic assay.

The affinity of the different DARPin molecules to Stx2a holotoxin using Biolayer Interferometry was also compared (BLI, FIGS. 6A-6G). Remarkably, the affinity of SHT (K_{Dapp}=27 nM) is 128-fold stronger than that of 10B (K_{Dapp}=3450 nM). The affinity of HT (K_{Dapp}=409 nM) is in between that of 10B and SHT. Although the trend of affinity improvement from 10B to SHT is the same in ELISA and BLI, the fold difference is drastically different. Without being bound by any theory, there could be a slight conformational difference between Stx2a-A1 (used in the ELISA) and Stx2a holotoxin (used in BLI) caused by either the protein tertiary structure or the immobilization condition. In addition, unmodified Stx2a-A1 was used to coat the ELISA plate directly while Stx2a with biotin molecules conjugated to random surface lysin residues was used in the BLI experiment.

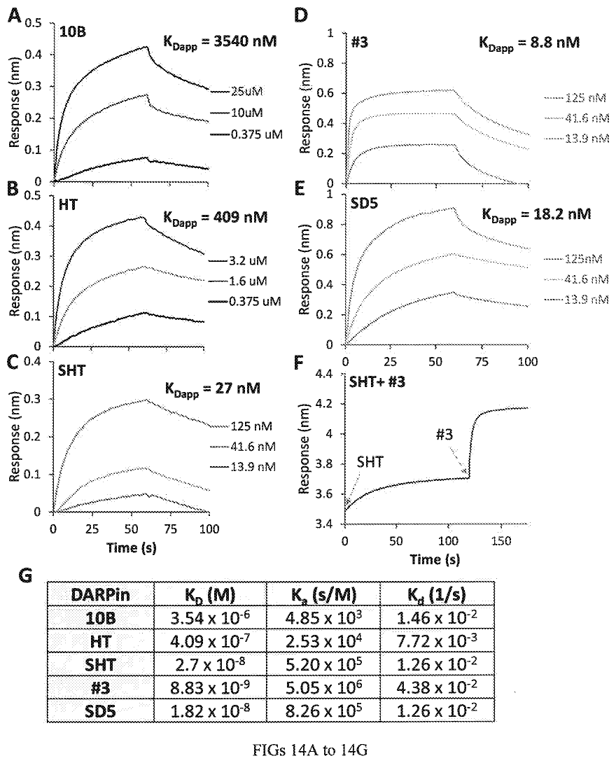

Since the other constituent of the DARPin dimer SD5 is DARPin #3, affinity of DARPin #3 and SD5 were also determined (FIGS. 14D, 14E). DARPin #3 binds Stx2a holotoxin with slightly higher affinity (K_{Dapp} 8.8 nM) than SHT. Interestingly, the affinity of dimer SD5 (K_{Dapp}=18.2 nM) falls in between its constituent DARPins. This result is somewhat unexpected because it was anticipated that the dimer exhibited even higher affinity through the avidity effect. Without being bound by any theory, the weaker affinity of SD5 may reflect some intramolecular hindrance between #3 and SHT or a reorientation of SD5 upon binding to Stx2a. The increased affinity of SHT over HT is contributed by both a faster Kₐ and a slower Kₐ (FIG. 14G), attesting the success of the off-rate selection.

To confirm that DARPin #3 and SHT can bind simultaneously to the same Stx2a molecule, a sequential binding experiment was carried out (FIG. 14F). An Stx2a-coated sensor was loaded first with SHT at a high concentration (i.e. 125 nM) to occupy all the binding sites on Stx2a before the loading of DARPin #3. The binding of SHT did not impede the binding of #3, indicating that SHT and #3 bind non-competitively to Stx2a.

### Example 5

### In Vivo Efficacy of DARPin

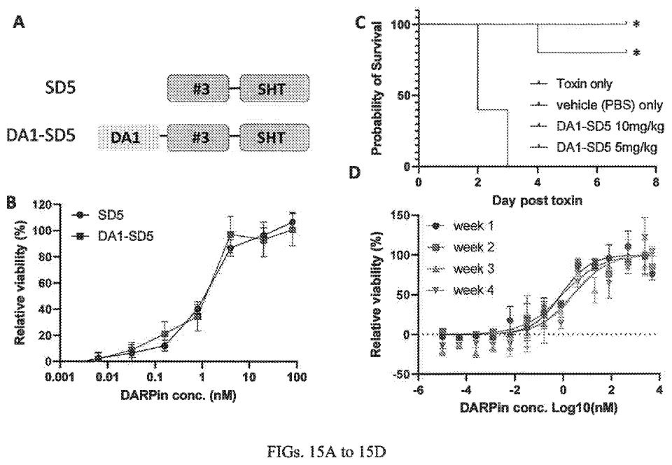

In the instant example, the in vivo efficacy of DARPin was explored. Due to its small size, 90% of monomer DARPin is excreted within 7.5 minutes in mice. Piggybacking onto albumin proteins has been shown to be an effective strategy to increase the half-life of therapeutic proteins by taking advantage of the long circulation half-life of serum albumin protein. Previously, a serum albumin-binding DARPin was engineered and found to significantly improve the circulation half-life of the fusion protein to 27-80 h in mouse and 2.6-20 days in cynomolgus monkey. To extend the in vivo half-life, DARPin SD5 was fused to an albumin-binding DARPin DA1 (FIG. 15A). The resulting DARPin DA1-SD5 exhibited identical Stx2a neutralization activity to the parent SD5 (FIG. 15B) indicating that DA1 fusion does not interfere with the SD5-toxin interaction.

To evaluate the in vivo efficacy, Swiss Webster mice (group of 5) were challenged intraperitoneally with either Stx2a (60 ng per mouse) alone or a mixture of Stx2a (60 ng) and DA1-SD5 (5 or 10 mg/kg) on day 0 (FIG. 15C). In the study, DARPin was pre-mixed with the toxin before injection. Mice were closely monitored after toxin challenge and were humanely euthanized when they exhibited neurologic signs, became less responsive to stimuli, or lose 20% of the body weight. All mice receiving the toxin alone died within 3 days. On the other hand, all mice receiving the same dose of Stx2a plus 10 mg/kg of DA1-SD5, and 4 out of the 5 mice receiving the toxin and 5 mg/kg of DA1-SD5, survived until the end of the study with no clinical sign of disease. Without being bound by any theory, these data offer an assessment of the in vivo efficacy of the engineered DA1-SD5. In particular, DARPin DA1-SD5 was shown to significantly protect mice from Stx2a intoxication (FIG. 15C). This is very different from the event of STEC infection in which toxins are gradually released from the bacteria within the GI tract and translocated to the blood stream.

Finally, the storage stability DA1-SD5 in PBS was assessed. After 4 weeks of storage at room temperature, negligible reduction of neutralization activity was observed (FIG. 15D), confirming the excellent shelf life of DARPin molecules. In summary, DA1-SD5 exhibited excellent storage stability, with virtually no activity loss after storage at room temperature for 4 weeks (FIG. 15D). This, combined with its ease of expression (up to 200 mg protein per liter of shaker-flask E. coli culture and 15/L in E. coli high cell-density fermentation), renders the anti-toxin DARPin highly attractive for clinical development. Although the toxin challenge model used here does not completely recapitulate STEC infection during which the toxin is secreted by the bacteria residing within the gut lumen, from where the toxin travels to the kidney upon translocation to cause renal damage, systemically delivered Stx2a induces similar renal damage phenotype as those observed during infection. This mouse toxin challenge model has been extensively used in preclinical evaluation of anti-toxins against Stx2 and has yielded outcomes consistent with the piglet infection model.

In summary, the engineering of a panel of DARPin molecules with potent Stx2a-neutralization activity is reported. The best DARPin, DA1-SD5, potently protects mice from Stx2a toxicity, exhibits excellent long-term stability at room temperature and has the potential to be manufactured at relatively low cost, rendering it an attractive candidate for further clinical development. In addition, the protein engineering approach combining directed evolution with rational design offers an effective method for engineering other binder proteins.

### Example 6

### Cryo-EM Study of DARPin #3 and Stx2a

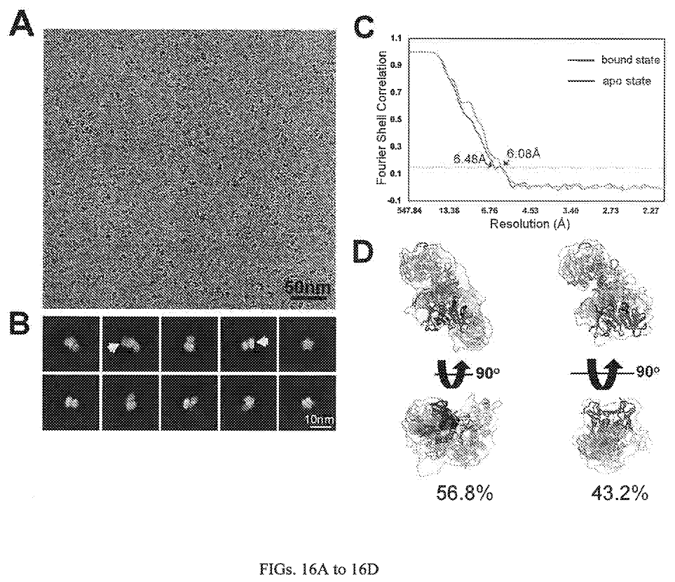

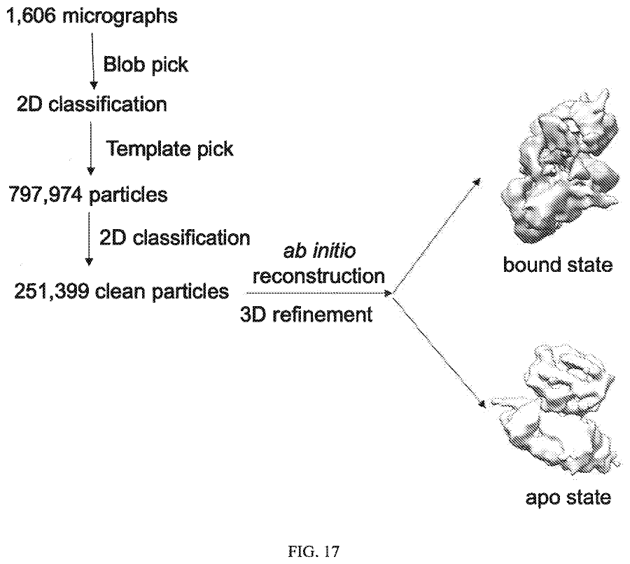

In the instant example, Cryo-EM study of DARPin #3 and Stx2a was explored. Since DARPin #3 failed to bind either Stx2a-A1 or Stx2a-B subunit in ELISA (FIGS. 5A-5D) despite its high affinity to the full-length toxin, cryo-EM was used to elucidate its epitope on Stx2a holotoxin. Reference-free 2D classification revealed multiple classes of Stx2a (FIG. 16A). Some classes lack any DARPin density and correspond to the apo state of Stx2a, while others clearly contain extra density that can only be attributed to the bound DARPin molecule (FIG. 16B). Heterogenous refinement confirmed that there are two particle groups: apo-state Stx2a and Stx2a-DARPin complex (FIG. 17). Both of the maps reach ˜6 Å, which allow us to dock the rigid domains of Stx2a (PDB: 1r4p) as well as DARPin into the density maps.

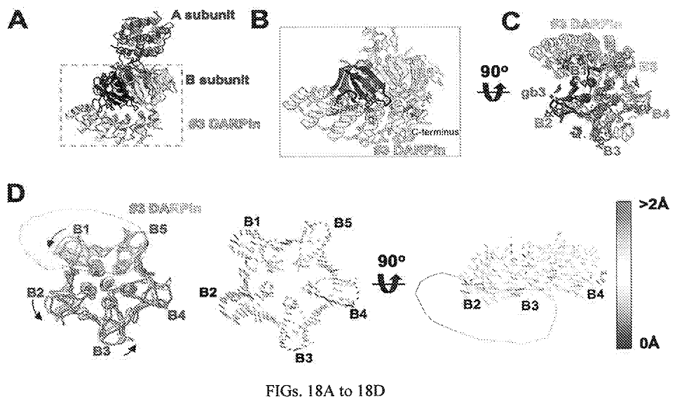

Based on the EM structure, DARPin #3 unequivocally binds the B-subunit at the basal side of Stx2a and interacts with two of the monomers in the B subunit pentamer (FIGS. 18A, 16B). Comparing to the apo state of Stx2a, the DARPin-bound Stx2a undergoes a conformational change in the B subunit. After aligning the corresponding A subunits between the apo state and the DARPin-bound state, a significant movement between the respective B subunit was observed. The B subunit closest to the C-terminus of DARPin #3 is defined as B1 while the remaining B subunits were named sequentially in a counterclockwise direction (FIG. 18C). Association of DARPin #3 induced a counterclockwise rotation of subunit B1, B2 and B3 (FIG. 18D, left), and an upward movement of subunit B4 (FIG. 18D, right), thus distorting the five-fold symmetry of the pentameric B subunit. The basal side of B-subunit is responsible for binding the carbohydrate moiety of the glycosphingolipid Gb₃ on the cell surface. DARPin #3 blocks the interaction pockets of monomer B1 and B5 and thus neutralizes the toxin.

Association of DARPin #3 induces a conformational change in the B-subunit that distorts its five-fold symmetry. Such a conformational change may not be possible in the purified Stx2a-B subunit lacking the A2 subunit. At the resolution available, it could not be discerned whether DARPin #3 also interacts with the A2 tail which protrudes from the B-subunit. Since the B-subunit adopts a five-fold symmetry, the fact that only one DARPin molecule was seen in association with the B-subunit may indicate the involvement of the A2 domain. This is in contrast to a previously developed Stx2e-neutralizing nanobody, NbStx2e1, which associates with the B subunit in such a way that five molecules are able to bind simultaneously to Stx2e without any steric hindrance. The ability of DARPin #3 to distort the tertiary structure of the B subunit is unprecedented and significant, and may derive from the structural rigidity of DARPins and an underappreciated structural flexibility of the toxin B subunit.

1. A dimer protein comprising a first monomer protein and a second monomer protein.

2. The dimer protein of claim 1, wherein the first monomer protein is the monomer protein of SEQ ID NO: 2.

3. The dimer protein of claim 1, wherein the second monomer protein is the monomer protein of SEQ ID NO: 49.

4. The dimer protein of claim 1, wherein the first monomer protein is the monomer protein of SEQ ID NO: 2 and the second monomer protein is the monomer protein of SEQ ID NO: 49.

5. The dimer protein of claim 1, wherein the first monomer protein and the second monomer protein are connected via a linker.

6. The dimer protein of claim 5, wherein the linker is selected from the group consisting of (GS)n, (GGGGS)n, (PS)n, and (PT)n, wherein n represents any number between 2-100.

7. The dimer protein of claim 5, wherein the linker is (G₄S)x4.

8. The dimer protein of claim 5, wherein the linker comprises SSSGGGGSEFGGGGSGGGGSGGGGSAS (SEQ ID NO: 73).

9. The dimer protein of any claim 5, wherein the linker consists essentially of SSSGGGGSEFGGGGSGGGGSGGGGSAS (SEQ ID NO: 73.

10. The dimer protein of claim 5, wherein the linker consists of SSSGGGGSEFGGGGSGGGGSGGGGSAS (SEQ ID NO: 73).

11.-71. (canceled)

72. A monomer protein comprising an amino acid sequence of SEQ ID NO: 41.

73.-84. (canceled)

85. A pharmaceutical composition comprising the dimer protein of claim 1.

86.-87. (canceled)

88. A method of treating a Shiga toxin-producing Escherichia coli (STEC) infection in a patient, said method comprising the step of administering a therapeutically effective amount of the pharmaceutical composition of claim 85 to the patient.

89. The method of claim 88, wherein the pharmaceutical composition reduces one or more symptoms of the STEC infection in the patient.

90. The method of claim 89, wherein the one or more symptoms comprise a gastrointestinal symptom.

91. The method of claim 89, wherein the one or more symptoms comprise diarrhea.

92. The method of claim 89, wherein the one or more symptoms comprise bloody diarrhea.

93. The method of claim 89, wherein the one or more symptoms comprise stomach cramps.

94. The method of claim 89, wherein the one or more symptoms comprise vomiting.

95. The method of claim 89, wherein the one or more symptoms comprise hemolytic uremic syndrome (HUS).
